(19) United States
(12) Patent Application Publication
(10) Pub. No.: US 2024/0002840 A1
(43) Pub. Date: Jan. 4, 2024

(54) NOVEL OMNI-50 CRISPR NUCLEASE-RNA COMPLEXES

(71) Applicants: Lior IZHAR, Tel Aviv (IL); EmendoBio Inc., Wilmington, DE (US)

(72) Inventors: Lior Izhar, Tel Aviv (IL); Liat Rockah, Rishon LeZion (IL); Nadav Marbach Bar, Rehovot (IL)

(73) Assignees: Liat ROCKAH, Rishon LeZion (IL); EmendoBio Inc., Wilmington, DE (US)

(21) Appl. No.: 18/251,667

(22) PCT Filed: Nov. 3, 2021
(86) PCT No.: PCT/US2021/057814

§ 371 Date: May 3, 2023

Related U.S. Application Data
(60) Provisional application No. 63/109,835, filed on Nov. 4, 2020.

(51) Int. Cl. C12N 15/11

(52) U.S. Cl. CPC C12N 15/11; C12N 2310/20

(57) ABSTRACT

A composition comprising a non-naturally occurring RNA molecule, the RNA molecule comprising an RNA scaffold portion, the RNA scaffold portion having the structure:
crRNA repeat sequence portion-Linker portion-tracrRNA portion;
   wherein the RNA scaffold portion forms a complex with and targets an OMNI-50 CRISPR nuclease to a DNA target site having complementarity to a guide sequence portion of the RNA molecule.

This application claims the benefit of U.S. Provisional Application No. 63/109,835, filed Nov. 4, 2020, the contents of which are hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### REFERENCE TO SEQUENCE LISTING

This application incorporates-by-reference nucleotide sequences which are present in the file named “211103 91628-A-PCT Sequence Listing AWG.txt”, which is 88 kilobytes in size, and which was created on Oct. 25, 2021 in the IBM-PC machine format, having an operating system compatibility with MS-Windows, which is contained in the text file filed Nov. 3, 2021 as part of this application.

### FIELD OF THE INVENTION

The present invention is directed to, inter alia, composition and methods for genome editing.

### BACKGROUND OF THE INVENTION

The Clustered Regularly Interspaced Short Palindromic Repeat (CRISPR) systems of bacterial and archaeal adaptive immunity show extreme diversity of protein composition and genomic loci architecture. The CRISPR systems have become important tools for research and genome engineering. Nevertheless, many details of CRISPR systems have not been determined and the applicability of CRISPR nucleases may be limited by sequence specificity requirements, expression, or delivery challenges. Different CRISPR nucleases have diverse characteristics such as: size, PAM site, on target activity, specificity, cleavage pattern (e.g. blunt, staggered ends), and prominent pattern of indel formation following cleavage. Different sets of characteristics may be useful for different applications. For example, some CRISPR nucleases may be able to target particular genomic loci that other CRISPR nucleases cannot due to limitations of the PAM site. In addition, some CRISPR nucleases currently in use exhibit pre-immunity, which may limit in vivo applicability. See Charlesworth et al., Nature Medicine (2019) and Wagner et al., Nature Medicine (2019). Accordingly, discovery, engineering, and improvement of novel CRISPR nucleases and the RNA molecules that activate and target them is of importance.

### SUMMARY OF THE INVENTION

The invention provides a composition comprising a non-naturally occurring RNA molecule, the RNA molecule comprising a crRNA repeat sequence portion and guide sequence portion, wherein the RNA molecule forms a complex with and targets an OMNI-50 nuclease to a DNA target site in the presence of a tracrRNA sequence, wherein the tracrRNA sequence is encoded by a tracrRNA portion of the RNA molecule or a tracrRNA portion of a second RNA molecule.

The invention also provides a composition comprising a non-naturally occurring RNA molecule, the RNA molecule comprising a crRNA repeat sequence portion, a guide sequence portion, and a tracrRNA portion, wherein the RNA molecule forms a complex with and targets an OMNI-50 nuclease to a DNA target site having complementarity to the guide sequence portion of the RNA molecule.

The invention also provides a composition comprising a non-naturally occurring RNA molecule, the RNA molecule comprising an RNA scaffold portion, the RNA scaffold portion having the structure:
crRNA repeat sequence portion-Linker portion-tracrRNA portion;
   wherein the RNA scaffold portion forms a complex with and targets an OMNI-50 CRISPR nuclease to a DNA target site having complementarity to a guide sequence portion of the RNA molecule.

Disclosed herein are compositions and methods that may be utilized for genomic engineering, epigenomic engineering, genome targeting, genome editing of cells, and/or in vitro diagnostics using an OMNI-50 CRISPR nuclease and a non-naturally occurring RNA molecule comprising a scaffold portion capable of specifically binding and activating the OMNI-50 CRISPR nuclease to target a DNA target site based on a guide sequence portion, also referred to as a RNA spacer portion, of the RNA molecule.

The disclosed compositions may be utilized for modifying genomic DNA sequences. As used herein, genomic DNA refers to linear and/or chromosomal DNA and/or plasmid or other extrachromosomal DNA sequences present in the cell or cells of interest. In some embodiments, the cell of interest is a eukaryotic cell. In some embodiments, the cell of interest is a prokaryotic cell. In some embodiments, the methods produce double-strand breaks (DSBs) at pre-determined target sites in a genomic DNA sequence, resulting in mutation, insertion, and/or deletion of a DNA sequence at the target site(s) in a genome.

### BRIEF DESCRIPTION OF THE DRAWINGS

FIGS. 1A-1G: The predicted secondary structures of the 3′ trimmed sgRNA listed in Table 3. FIG. 1A: “Full c” RNA structure. FIG. 1B: “Short 1” RNA structure. FIG. 1C: “Short 2” RNA structure. FIG. 1D: “Short 3” RNA structure. FIG. 1E: “Short 4” RNA structure. FIG. 1F: “Short 5” RNA structure. FIG. 1G: “Short 6” RNA structure.

FIG. 2: Activity assay of 3′ trimmed guides (Table 3) in RNPs. Purified OMNI-50 was reacted with IVT transcribed shorted guides. For the in vitro assays, the RNPs were reacted with 5′ -FAM labelled linear substrate. Cleavage efficiency was calculated by the fluorescence of cut fragment divided by the sum of the cut and uncut fragments. For the in vivo assays U2OS cells were electroporated with RNP, and activity was determined as indel frequency by NGS.

FIGS. 3A-3M: The predicted secondary structures of the full scaffold sgRNA version f and version c as listed in Table 4 and the high ranked short sgRNA listed in Table 5. FIG. 3A: “Full f” RNA structure. FIG. 3B: “Full c” RNA structure. FIG. 3C: “NGS13” RNA structure. FIG. 3D: “NGS14” RNA structure. FIG. 3E: “NGS15” RNA structure. FIG. 3F: “NGS16” RNA structure. FIG. 3G: “NGS17” RNA structure. FIG. 3H: “NGS18” RNA structure. FIG. 3I: “NGS40” RNA structure. FIG. 3J: “NGS41” RNA structure. FIG. 3K: “NGS42” RNA structure. FIG. 3L: “NGS43” RNA structure. FIG. 3M: “NGS44” RNA structure.

FIGS. 4A-4F: The predicted secondary structures of the medium ranked short sgRNA listed in Table 6. FIG. 4A: “NGS9” RNA structure. FIG. 4B: “NGS2” RNA structure. FIG. 4C: “NGS3” RNA structure. FIG. 4D: “NGS12” RNA structure. FIG. 4E: “NGS1” RNA structure. FIG. 4F: “NGS6” RNA structure.

FIGS. 5A-5E: RNP activity in U2OS mammalian cell line with permissive guides (g35, g62). U2OS cells were electroporated with RNP of OMNI-50 with the indicated sgRNAs and activity was determined as indel frequency on the targeted protospacers and their known off targets by NGS (g35, g62, Table 8, Table 9). FIG. 5A: “g35-ON” or “g35-OT2” guide (spacer) with a NGS1, NGS2, NGS3, NGS6, or NGS9 scaffold, as well as “No treatment” (NT) and “No guide” controls. FIG. 5B: g35 On-target or g35 Off-target guide (spacer) with a NGS1, NGS12, NGS13, NGS14, NGS15, NGS16, NGS17, NGS18, NGS2, NGS3, NGS6, or NGS9 scaffold, as well as a “No treatment” (NT) control. FIG. 5C: “g35-ON” or “g35-OT2” guide (spacer) with a NGS1, NGS6, NGS12, NGS17, or Full f scaffold, as well as a “No treatment” (NT) control. FIG. 5D: “g62-ON”, “g62-OT1”, or “g62-OT2” guide (spacer) with a Full for NGS1 scaffold, as well as “No treatment” (NT) and “No guide” controls. FIG. 5E: “g62-ON”, “g62-OT1”, or “g62-OT2” guide (spacer) with a NGS9 or NGS17 scaffold, as well as “No guide” and “No treatment” (NT) controls.

FIGS. 6A-6C: Activity in U2OS mammalian cell line with challenging guide (g58). U2OS cells were electroporated with RNP of OMNI-50 with the indicated sgRNA and activity was determined as indel frequency on the targeted protospacer and its known off targets by NGS (g58, Table 8, Table 9). FIG. 6A: “g58-ON” or “g58-OT2” guide (spacer) with a Full for NGS1 scaffold, as well as “No treatment” (NT) and “No guide” controls. FIG. 6B: “g58-ON” or “g58-OT2” guide (spacer) with a NGS12, NGS9, or NGS17 scaffold, as well as “No treatment” (NT) and “No guide” controls. FIG. 6C: “g58” or “g58-OT” guide (spacer) with a Full f, NGS12, NGS,40, NGS41, NGS42, NGS43, or NGS44 scaffold, as well as “No treatment” (NT) and “No guide” controls.

FIGS. 7A-7B: Activity in HSC500 and LCL cells with short sgRNA. FIG. 7A: HSC500 cells were electroporated with RNP of OMNI-50 with the indicated sgRNAs and activity was determined as indel frequency on the targeted protospacer and its known off targets by NGS (g58 and g35, Table 8, Table 9). FIG. 7B: LCL cells were electroporated with RNP of OMNI-50 with the indicated sgRNAs and activity was determined as indel frequency on the targeted protospacer and its known off targets by NGS (g58 and g35, Table 8, Table 9).

FIG. 8: Representative example of an RNA scaffold. An example RNA scaffold portion comprises a crRNA portion linked by a tetraloop to a tracrRNA portion. The crRNA portion comprises a crRNA repeat sequence. The tracrRNA portion comprises a tracrRNA anti-repeat sequence and additional tracrRNA sections. The RNA molecule may further comprise a guide sequence portion (i.e. an RNA spacer) linked to the crRNA repeat sequence, such that the RNA molecule functions as a single-guide RNA molecule.

### DETAILED DESCRIPTION

This invention provides a composition comprising a non-naturally occurring RNA molecule, the RNA molecule comprising a crRNA repeat sequence portion and a guide sequence portion, wherein the RNA molecule forms a complex with and targets an OMNI-50 nuclease to a DNA target site in the presence of a tracrRNA sequence, wherein the tracrRNA sequence is encoded by a tracrRNA portion of the RNA molecule or a tracrRNA portion of a second RNA molecule.

The RNA molecule comprising a crRNA repeat sequence portion and a guide sequence portion and the tracrRNA molecule may be separate molecules. Alternatively, the RNA molecule comprising a crRNA repeat sequence portion and a guide sequence portion may be linked to the tracrRNA molecule to form a single RNA molecule having a crRNA repeat sequence portion, a guide sequence portion, and a tracrRNA portion.

In some embodiments, the crRNA repeat sequence portion is less than 17 nucleotides in length, preferably 12-16 nucleotides in length, or the crRNA repeat sequence portion is 17 or more nucleotides in length, preferably 18-24 nucleotides in length.

In some embodiments, the crRNA repeat sequence portion has at least 60-70%, 71-80%, 81-90%, 91-95%, or 96-99% sequence identity to a mature OMNI-50 crRNA repeat sequence encoded by Ezakiella peruensis strain M6.X2.

In some embodiments, the crRNA repeat sequence portion at least 60-70%, 71-80%, 81-90%, 91-95%, or 96-99% sequence identity to SEQ ID NO: 23.

In some embodiments, the crRNA repeat sequence portion has at least 95% sequence identity to any one of SEQ ID NOs: 8, 23, 24, and 25.

In some embodiments, the crRNA repeat sequence is other than SEQ ID NO: 8 or 23.

In some embodiments, the RNA molecule comprising a crRNA repeat sequence portion and a guide sequence portion further comprises a tracrRNA portion.

In some embodiments, the crRNA repeat sequence portion is covalently linked to the tracrRNA portion by a polynucleotide linker portion.

In some embodiments, the composition further comprises a second RNA molecule comprising a tracrRNA portion.

In some embodiments, the OMNI-50 nuclease has at least 95% sequence identity to the amino acid sequence of SEQ ID NO: 1.

In some embodiments, the guide sequence portion is 17-30 nucleotides in length, preferably 22 nucleotides in length.

The invention also provides a composition comprising a non-naturally occurring RNA molecule, the RNA molecule comprising a tracrRNA portion, wherein the RNA molecule forms a complex with and targets an OMNI-50 nuclease to a DNA target site in the presence of a crRNA repeat sequence portion and a guide sequence portion, wherein the crRNA repeat sequence portion and the guide sequence portion are encoded by the RNA molecule or a second RNA molecule.

The RNA molecule comprising a crRNA repeat sequence portion and a guide sequence portion and the RNA molecule comprising a tracrRNA portion may be separate molecules. Alternatively, the RNA molecule comprising a crRNA repeat sequence portion and a guide sequence portion may be linked to the tracrRNA molecule to form a single RNA molecule having a crRNA repeat sequence portion, a guide sequence portion, and a tracrRNA portion.

In some embodiments, the tracrRNA portion is less than 91 nucleotides in length, preferably 90-80, 89-80, 79-70, 69-60, 59-50, 49-40, or 39-28 nucleotides in length, or the tracrRNA portion is 91 or more nucleotides in length, preferably 91-112 nucleotides in length.

In some embodiments, the tracrRNA portion has at least 30-40%, 41-50%, 51-60%, 61-70%, 71-80%, 81-90%, 91-95%, or 96-99% sequence identity to a mature OMNI-50 tracrRNA sequence encoded by Ezakiella peruensis strain M6.X2.

In some embodiments, the tracrRNA portion has at least 30-40%, 41-50%, 51-60%, 61-70%, 71-80%, 81-90%, 91-95%, or 96-99% sequence identity to the tracrRNA portion of SEQ ID NO: 5.

In some embodiments, the tracrRNA portion has at least 95% sequence identity to the tracrRNA portion of any one of SEQ ID NOs: 4, 5, 16-21, 29-31, 74-126, 132-137, and 148-167.

In some embodiments, the tracrRNA portion is other than the tracrRNA portion of SEQ ID NO: 4 or 5.

In some embodiments, the tracrRNA portion comprises a tracrRNA anti-repeat sequence portion that is less than 19 nucleotides in length, preferably 14-18 nucleotides in length.

In some embodiments, the tracrRNA portion comprises a tracrRNA anti-repeat sequence portion that has at least 60-70%, 71-80%, 81-90%, 91-95%, or 96-99% sequence identity to SEQ ID NO: 26.

In some embodiments, the tracrRNA portion comprises a tracrRNA anti-repeat sequence portion that has at least 95% sequence identity to any one of SEQ ID NOs: 9, 26-28, and 138.

In some embodiments, the tracrRNA portion comprises a tracrRNA anti-repeat sequence portion is other than SEQ ID NO: 9 or 26.

In some embodiments, the RNA molecule comprises a tracrRNA portion and further comprises a crRNA repeat sequence portion and a guide sequence portion.

In some embodiments, the tracrRNA portion is covalently linked to the crRNA repeat sequence by a polynucleotide linker portion.

In some embodiments, the polynucleotide linker portion is 4-10 nucleotides in length.

In some embodiments, the polynucleotide linker has a sequence of GAAA.

In some embodiments, the composition further comprises a second RNA molecule comprising a crRNA repeat sequence portion and a guide sequence portion.

In some embodiments, the OMNI-50 nuclease has at least 95% sequence identity to the amino acid sequence of SEQ ID NO: 1.

In some embodiments, the guide sequence portion is 17-30 nucleotides in length, preferably 22 nucleotides in length.

The invention provides a composition comprising a non-naturally occurring RNA molecule, the RNA molecule comprising an RNA scaffold portion, the RNA scaffold portion having the following structure:
crRNA repeat sequence portion-Linker portion-tracrRNA portion;
wherein the RNA scaffold portion froms a complex with and targets an OMNI-50 CRISPR nuclease to a DNA target site having complimentarity to a guide sequence portion of the RNA molecule.

In some embodiments, the RNA scaffold portion is 112, 111-110, 109-105, 104-100, 99-94-90, 89-85, 84-80, 79-75, 74-70, 69-50, or 49-45 nucleotides in length.

In some embodiments, the RNA scaffold portion has at least 30%, at least 40%, at least 50%, at least 60%, at least 70%, at least 80%, at least 85%, at least 90%, or at least 95% sequence identity to SEQ ID NO: 5.

In some embodiments, the crRNA repeat sequence portion is less than 17 nucleotides in length, preferably 12-16 nucleotides in length, or the crRNA repeat sequence portion is 17 or more nucleotides in length, preferably 18-24 nucleotides in length.

In some embodiments, the crRNA repeat sequence portion has at least 60-70%, 71-80%, 81-90%, 91-95%, or 96-99% sequence identity to a mature OMNI-50 crRNA repeat sequence encoded by Ezakiella peruensis strain M6.X2.

In some embodiments, the crRNA repeat sequence portion at least 60-70%, 71-80%, 81-90%, 91-95%, or 96-99% sequence identity to SEQ ID NO: 23.

In some embodiments, the crRNA repeat sequence portion has at least 95% sequence identity to any one of SEQ ID NOs: 8, 23, 24, and 25.

In some embodiments, the crRNA repeat sequence is other than SEQ ID NO: 8 or 23.

In some embodiments, the tracrRNA portion is less than 91 nucleotides in length, preferably 90-80, 89-80, 79-70, 69-60, 59-50, 49-40, or 39-28 nucleotides in length, or the tracrRNA portion is 91 or more nucleotides in length, preferably 91-112 nucleotides in length.

In some embodiments, the tracrRNA portion has at least 30-40%, 41-50%, 51-60%, 61-70%, 71-80%, 81-90%, 91-95%, or 96-99% sequence identity to a mature OMNI-50 tracrRNA sequence encoded by Ezakiella peruensis strain M6.X2.

In some embodiments, the tracrRNA portion has at least 30-40%, 41-50%, 51-60%, 61-70%, 71-80%, 81-90%, 91-95%, or 96-99% sequence identity to the tracrRNA portion of SEQ ID NO: 5.

In some embodiments, the tracrRNA portion has at least 95% sequence identity to the tracrRNA portions of any one of SEQ ID NOs: 4, 5, 16-21, 29-31, 74-126, 132-137, and 148-167.

In some embodiments, the tracrRNA portion is other than the tracrRNA portion of SEQ ID NO: 4 or 5.

In some embodiments, the tracrRNA portion comprises a tracrRNA anti-repeat sequence portion, wherein the crRNA repeat sequence and the tracrRNA anti-repeat sequence portion are covalently linked by the linker portion.

In some embodiments, the linker portion is a polynucleotide linker that is 4-10 nucleotides in length.

In some embodiments, the polynucleotide linker has a sequence of GAAA.

In some embodiments, the tracrRNA anti-repeat sequence portion is less than 19 nucleotides in length, preferably 14-18 nucleotides in length.

In some embodiments, the tracrRNA portion comprises a tracrRNA anti-repeat sequence portion that has at least 60-70%, 71-80%, 81-90%, 91-95%, or 96-99% sequence identity to SEQ ID NO: 5.

In some embodiments, the tracrRNA anti-repeat sequence portion has at least 95% sequence identity to any one of SEQ ID NOs: 9, 26-28, and 138.

In some embodiments, the tracrRNA anti-repeat sequence portion is other than SEQ ID NO: 9 or 26.

In some embodiments, the tracrRNA portion comprises a first section of nucleotides linked to the tracrRNA anti-repeat portion, and the first section of nucleotides has at least 95% sequence identity to any one of SEQ ID NOs: 10, 11, 127, 128, 139-143, AAC, A, AA, AAA, and ACAAACC.

In some embodiments, the tracrRNA portion comprises a second section of nucleotides linked to a first section of nucleotides, and the second section of nucleotides has at least 95% sequence identity to any one of SEQ ID NOs: 12, 32, 144-146, GCCUAUU, GCCUAU, AAUGGC, AAAGGC, UAUAGGC, AUAGGC, and GCCU.

In some embodiments, the tracrRNA portion comprises a third section of nucleotides linked to a second section of nucleotides, and the third section of nucleotides has at least 95% sequence identity to any one of SEQ ID NOs: 13, 33, 34, 129, 130, 147, CGCAG, CGC, CGCAGG, C, CUUCUGC, and CGCAGUUG.

In some embodiments, the tracrRNA portion comprises a fourth portion of nucleotides linked to a third section of nucletides, and the fourth section of nucleotides has at least 95% sequence identity to any one of SEQ ID NOs: 14, 15, 35-73, 131, AUU, AUUAUUU, AUUU, AUUUUUUUU, AGCUUUUUU, UUUU, UUUUU, and UUU.

In some embodiments, the RNA scaffold portion has at least 95% identity to the nucleotide sequence of SEQ ID NO: 4, 5, 16-21, 29-31, 74-126, 132-137, and 148-167.

In some embodiments, the RNA scaffold portion has a predicted structure of any one of the Full F, Full C, Short 1, Short 2, Short 3, Short 4, Short 5, Short 6, NGS13, NGS14, NGS15, NGS16, NGS17, NGS18, NGS40, NGS41, NGS42, NGS43, NGS44, NGS9, NGS2, NGS3, NGS12, NGS1, or NGS6 RNA scaffolds.

In some embodiments, the RNA scaffold portion is other than SEQ ID NO: 4 or 5.

In some embodiments, the guide sequence portion is covalently linked to the crRNA repeat sequence portion of the RNA molecule, forming a single-guide RNA molecule having a structure:
Guide sequence portion-crRNA repeat sequence portion-Linker portion-tracrRNA portion.

In some embodiments, the guide sequence portion is 17-30 nucleotides, more preferably 20-23 nucleotides, more preferably 22 nucleotides in length.

In some embodiments, the composition further comprises an OMNI-50 CRISPR nuclease, wherein the OMNI-50 CRISPR nuclease has at least 95% identity to the amino acid sequence of SEQ ID NO: 1.

In some embodiments, the RNA molecule is formed by in vitro transcription (IVT) or solid-phase artificial oligonucleotide synthesis.

In some embodiments, the RNA molecule comprises modified nucleotides.

The invention also provides a polynucleotide molecule encoding the RNA molecule of any one of the embodiments described herein.

The present invention provides compositions which comprise at least one non-naturally occurring RNA molecule that specifically binds, activates, and/or targets an OMNI-50 nuclease.

The invention also provides a method of modifying a nucleotide sequence at a DNA target site in a cell-free system or a genome of a cell comprising introducing into the system or cell the composition of any one of the embodiments described herein and an OMNI-50 CRISPR nuclease. The DNA target site is determined by an RNA spacer encoded by an RNA molecule of the composition, such that the RNA spacer is complementary in sequence to the DNA target site.

In some embodiments, the cell is a eukaryotic cell or a prokaryotic cell.

The invention also provides a kit for modifying a nucleotide sequence at a DNA target site in a cell-free system or a genome of a cell comprising introducing into the system or cell the composition of any one of the embodiments described herein, an OMNI-50 CRISPR nuclease, and instructions for delivering the composition and the OMNI-50 CRISPR nuclease to the cell.

In some embodiments, the non-naturally occurring RNA molecule comprises a crRNA sequence portion that differs from the wild-type crRNA sequence of Ezakiella peruensis strain M6.X2. In some embodiments, the non-naturally occurring RNA molecule comprises a crRNA sequence portion that is shorter than the wild-type crRNA sequence of Ezakiella peruensis strain M6.X2.

In some embodiments, the non-naturally occurring RNA molecule comprises a tracrRNA sequence portion that differs from the wild-type tracrRNA sequence of Ezakiella peruensis strain M6.X2. In some embodiments, the non-naturally occurring RNA molecule comprises a tracrRNA sequence portion that is shorter than the wild-type tracrRNA sequence of Ezakiella peruensis strain M6.X2.

In embodiments of the present invention, the non-naturally occurring RNA molecule comprises a “spacer” or “guide sequence” portion. The “spacer portion” or “guide sequence portion” of an RNA molecule refers to a nucleotide sequence that is capable of hybridizing to a specific target DNA sequence, e.g., the guide sequence portion has a nucleotide sequence which is fully complementary to the DNA sequence being targeted along the length of the guide sequence portion. In some embodiments, the guide sequence portion is 17, 18, 19, 20, 21, 22, 23, 24, 25, 26, 27, 28, 29, or 30 nucleotides in length, or approximately 17-30, 17-29, 17-28, 17-27, 17-26, 17-25, 17-24, 18-22, 19-22, 18-20, 17-20, or 21-22 nucleotides in length. Preferably, the entire length of the guide sequence portion is fully complementary to the DNA sequence being targeted along the length of the guide sequence portion. The guide sequence portion may be part of an RNA molecule having a “scaffold portion” that can form a complex with and activate a CRISPR nuclease, with the guide sequence portion of the RNA molecule serving as the DNA targeting portion of the CRISPR complex. When the RNA molecule having a scaffold portion and a guide sequence portion is present contemporaneously with the CRISPR molecule, the RNA molecule is capable of targeting the CRISPR nuclease to the specific target DNA sequence. Each possibility represents a separate embodiment. The RNA molecule spacer portion can be custom designed to target any desired sequence.

In an embodiment, the nuclease-binding RNA nucleotide sequence and the DNA-targeting RNA nucleotide sequence (e.g. spacer or guide sequence portion) are on a single-guide RNA molecule (sgRNA), wherein the sgRNA molecule can form a complex with the OMNI-50 CRISPR nuclease and serve as the DNA targeting module.

In an embodiment, the nuclease-binding RNA nucleotide sequence is on a first RNA molecule and the DNA-targeting RNA nucleotide sequence is on a second RNA molecule, and the first and second RNA molecules interact by base-pairing and complex with the CRISPR nuclease to serve as the targeting module.

According to some aspects of the invention, the disclosed methods comprise a method of modifying a nucleotide sequence at a target site in a cell-free system or the genome of a cell comprising introducing into the cell the composition of any one of the embodiments described herein.

This invention also provides use of any of the compositions or methods of the invention for modifying a nucleotide sequence at a DNA target site in a cell.

This invention provides a method of modifying a nucleotide sequence at a target site in the genome of a eukaryotic cell.

This invention provides a method of modifying a nucleotide sequence at a target site in the genome of a mammalian cell.

This invention provides a method of modifying a nucleotide sequence at a target site in the genome of a plant cell.

In some embodiments, the method is performed ex vivo. In some embodiments, the method is performed in vivo. In some embodiments, some steps of the method are performed ex vivo and some steps are performed in vivo. In some embodiments the mammalian cell is a human cell.

This invention also provides a modified cell or cells obtained by any of the methods described herein. In an embodiment these modified cell or cells are capable of giving rise to progeny cells. In an embodiment these modified cell or cells are capable of giving rise to progeny cells after engraftment.

This invention also provides a composition comprising these modified cells and a pharmaceutically acceptable carrier. Also provided is an in vitro or ex vivo method of preparing this, comprising mixing the cells with the pharmaceutically acceptable carrier.

According to some aspects of the invention, the disclosed methods comprise a use of any one of the compositions described herein for the treatment of a subject afflicted with a disease associated with a genomic mutation comprising modifying a nucleotide sequence at a target site in the genome of the subject.

According to some aspects of the invention, the disclosed methods comprise a method of treating subject having a mutation disorder comprising targeting any one of the compositions described herein to an allele associated with the mutation disorder.

In some embodiments, the mutation disorder is related to a disease or disorder selected from any of a neoplasia, age-related macular degeneration, schizophrenia, neurological, neurodegenerative, or movement disorder, Fragile X Syndrome, secretase-related disorders, prion-related disorders, ALS, addiction, autism, Alzheimer's Disease, neutropenia, inflammation-related disorders, Parkinson's Disease, blood and coagulation diseases and disorders, cell dysregulation and oncology diseases and disorders, inflammation and immune-related diseases and disorders, metabolic, liver, kidney and protein diseases and disorders, muscular and skeletal diseases and disorders, dermatological diseases and disorders, neurological and neuronal diseases and disorders, and ocular diseases and disorders.

### Diseases and Therapies

Certain embodiments of the invention target a nuclease to a specific genetic locus associated with a disease or disorder as a form of gene editing, method of treatment, or therapy. For example, to induce editing or knockout of a gene, a composition disclosed herein may be specifically targeted to a pathogenic mutant allele of the gene using a custom designed guide RNA molecule. The guide RNA molecule is preferably designed by first considering the PAM requirement of the nuclease, which as shown herein is also dependent on the system in which the gene editing is being performed. For example, a guide RNA molecule designed to target an OMNI-50 nuclease to a target site is designed to contain a spacer region complementary to a region neighboring the OMNI-50 PAM sequence “NGG.” The guide RNA molecule is further preferably designed to contain a spacer region (i.e. the region of the guide RNA molecule having complementarity to the target allele) of sufficient and preferably optimal length in order to increase specific activity of the nuclease and reduce off-target effects. For example, a guide RNA molecule designed to target OMNI-50 nuclease may be designed to contain a 22-nucleotide spacer for high on-target cleavage activity.

As a non-limiting example, the guide RNA molecule may be designed to target the nuclease to a specific region of a mutant allele, e.g. near the start codon, such that upon DNA damage caused by the nuclease a non-homologous end joining (NHEJ) pathway is induced and leads to silencing of the mutant allele by introduction of frameshift mutations. This approach to guide RNA molecule design is particularly useful for altering the effects of dominant negative mutations and thereby treating a subject. As a separate non-limiting example, the guide RNA molecule may be designed to target a specific pathogenic mutation of a mutated allele, such that upon DNA damage caused by the nuclease a homology directed repair (HDR) pathway is induced and leads to template mediated correction of the mutant allele. This approach to guide RNA molecule design is particularly useful for altering haploinsufficiency effects of a mutated allele and thereby treating a subject.

Non-limiting examples of specific genes which may be targeted for alteration to treat a disease or disorder are presented herein below. Specific disease-associated genes and mutations that induce a mutation disorder are described in the literature. Such mutations can be used to design a DNA-targeting RNA molecule to target a CRISPR composition to an allele of the disease associated gene, where the CRISPR composition causes DNA damage and induces a DNA repair pathway to alter the allele and thereby treat the mutation disorder.

Mutations in the ELANE gene are associated with neutropenia. Accordingly, without limitation, embodiments of the invention that target ELANE may be used in methods of treating subjects afflicted with neutropenia.

CXCR4 is a co-receptor for the human immunodeficiency virus type 1 (HIV-1) infection. Accordingly, without limitation, embodiments of the invention that target CXCR4 may be used in methods of treating subjects afflicted with HIV-1 or conferring resistance to HIV-1 infection in a subject.

Programmed cell death protein 1 (PD-1) disruption enhances CAR-T cell mediated killing of tumor cells and PD-1 may be a target in other cancer therapies. Accordingly, without limitation, embodiments of the invention that target PD-1 may be used in methods of treating subjects afflicted with cancer. In an embodiment, the treatment is CAR-T cell therapy with T cells that have been modified according to the invention to be PD-1 deficient.

In addition, BCL11A is a gene that plays a role in the suppression of hemoglobin production. Globin production may be increased to treat diseases such as thalassemia or sickle cell anemia by inhibiting BCL11A. See for example, PCT International Publication No. WO 2017/077394A2; U.S. Publication No. US2011/0182867A1; Humbert et al. Sci. Transl. Med. (2019); and Canver et al. Nature (2015). Accordingly, without limitation, embodiments of the invention that target an enhancer of BCL11A may be used in methods of treating subjects afflicted with beta thalassemia or sickle cell anemia.

Embodiments of the invention may also be used for targeting any disease-associated gene, for studying, altering, or treating any of the diseases or disorders listed in Table A or Table B below. Indeed, any disease-associated with a genetic locus may be studied, altered, or treated by using the nucleases disclosed herein to target the appropriate disease-associated gene, for example, those listed in U.S. Publication No. 2018/0282762A1 and European Patent No. EP3079726B1.

TABLE A
Diseases, Disorders and their associated genes

| DISEASE/DISORDERS | GENE(S) |
|---|---|
| Neoplasia | PTEN; ATM; ATR; EGFR; ERBB2; ERBB3; ERBB4; Notch1; |
| | Notch2; Notch3; Notch4; AKT; AKT2; AKT3; HIF; HIF1a; |
| | HIF3a; Met; HRG; Bcl2; PPAR alpha; PPAR gamma; WT1 |
| | (Wilms Tumor); FGF Receptor Family members (5 members: 1, |
| | 2, 3, 4, 5); CDKN2a; APC; RB (retinoblastoma); MEN1; VHL; |
| | BRCA1; BRCA2; AR (Androgen Receptor); TSG101; IGF; IGF |
| | Receptor; Igf1 (4 variants); gf2 (3 variants); Igf 1 Receptor; Igf 2 |
| | Receptor; Bax; Bcl2; caspases family (9 members: 1, 2, 3, 4, 6, |
| | 7, 8, 9, 12); Kras; Apc |
| Age-related Macular | Abcr; Ccl2; Cc2; cp (ceruloplasmin); Timp3; cathepsinD; Vldlr; |
| Degeneration | Ccr2 |
| Schizophrenia | Neuregulin1 (Nrg1); Erb4 (receptor for Neuregulin); |
| | Complexin1 (Cp1x1); Tph1 Tryptophan hydroxylase; Tph2 |
| | Tryptophan hydroxylase 2; Neurexin 1; GSK3; GSK3a; GSK3b |
| Neurological, Neuro | 5-HTT (S1c6a4); COMT; DRD (Drd1a); SLC6A3; DAOA; |
| degenerative, and | DTNBP1; Dao (Dao1) |
| Movement Disorders | |
| Trinucleotide Repeat | HTT (Huntington's Dx); SBMA/SMAX1/AR (Kennedy's Dx); |
| Disorders | FXN/X25 (Friedrich's Ataxia); ATX3 (Machado-Joseph's Dx); |
| | ATXN1 and ATXN2 (spinocerebellar ataxias); DMPK |
| | (myotonic dystrophy); Atrophin-1 and Atn1 (DRPLA Dx); CBP |
| | (Creb-BP - global instability); VLDLR (Alzheimer's); Atxn7; |
| | Atxn10 |
| Fragile X Syndrome | FMR2; FXR1; FXR2; mGLUR5 |
| Secretase Related | APH-1 (alpha and beta); Presenilin (Psen1); nicastrin (Ncstn); |
| Disorders | PEN-2 |
| Others | Nos1; Parp1; Nat1; Nat2 |
| Prion related disorders | Prp |
| ALS | SOD1; ALS2; STEX; FUS; TARDBP; VEGF (VEGF-a; VEGF- |
| | b; VEGF-c) |
| Addiction | Prkce (alcohol); Drd2; Drd4; ABAT (alcohol); GRIA2; Grm5; |
| | Grin1; Htr1b; Grin2a; Drd3; Pdyn; Gria1 (alcohol) |
| Autism | Mecp2; BZRAP1; MDGA2; Sema5A; Neurexin 1; Fragile X |
| | (FMR2 (AFF2); FXR1; FXR2; Mglur5) |
| Alzheimer's Disease | E1; CHIP; UCH; UBB; Tau; LRP; PICALM; Clusterin; PS1; |
| | SORL1; CR1; Vldlr; Uba1; Uba3; CHIP28 (Aqp1, Aquaporin |
| | 1); Uchl1; Uchl3; APP |
| Inflammation | IL-10; IL-1 (IL-1a; IL-1b); IL-13; IL-17 (IL-17a (CTLA8); IL- |
| | 17b; IL-17c; IL-17d; IL-17f); II-23; Cx3cr1; ptpn22; TNFa; |
| | NOD2/CARD15 for IBD; IL-6; IL-12 (IL-12a; IL-12b); CTLA4; |
| | Cx3cl1 |
| Parkinson's Disease | x-Synuclein; DJ-1; LRRK2; Parkin; PINK1 |

TABLE B
Diseases, Disorders and their associated genes

| DISEASE CATEGORY | DISEASE AND ASSOCIATED GENES |
|---|---|
| Blood and coagulation | Anemia (CDAN1, CDA1, RPS19, DBA, PKLR, PK1, NT5C3, |
| diseases and disorders | UMPH1, PSN1, RHAG, RH50A, NRAMP2, SPTB, ALAS2, |
| | ANH1, ASB, ABCB7, ABC7, ASAT); Bare lymphocyte |
| | syndrome (TAPBP, TPSN, TAP2, ABCB3, PSF2, RING11, |
| | MHC2TA, C2TA, RFX5, RFXAP, RFX5), Bleeding disorders |
| | (TBXA2R, P2RX1, P2X1); Factor H and factor H-like 1 (HF1, |
| | CFH, HUS); Factor V and factor VIII (MCFD2); Factor VII |
| | deficiency (F7); Factor X deficiency (F10); Factor XI deficiency |
| | (F11); Factor XII deficiency (F12, HAF); Factor XIIIA |
| | deficiency (F13A1, F13A); Factor XIIIB deficiency (F13B); |
| | Fanconi anemia (FANCA, FACA, FA1, FA, FAA, FAAP95, |
| | FAAP90, FLJ34064, FANCB, FANCC, FACC, BRCA2, |
| | FANCD1, FANCD2, FANCD, FACD, FAD, FANCE, FACE, |
| | FANCF, XRCC9, FANCG, BRIP1, BACH1, FANCJ, PHF9, |
| | FANCL, FANCM, KIAA1596); Hemophagocytic |
| | lymphohistiocytosis disorders (PRF1, HPLH2, UNC13D, |
| | MUNC13-4, HPLH3, HLH3, FHL3); Hemophilia A (F8, F8C, |
| | HEMA); Hemophilia B (F9, HEMB), Hemorrhagic disorders |
| | (PI, ATT, F5); Leukocyde deficiencies and disorders (ITGB2, |
| | CD18, LCAMB, LAD, EIF2B1, EIF2BA, EIF2B2, EIF2B3, |
| | EIF2B5, LVWM, CACH, CLE, EIF2B4); Sickle cell anemia |
| | (HBB); Thalassemia (HBA2, HBB, HBD, LCRB, HBA1) |
| Cell dysregulation and | B-cell non-Hodgkin lymphoma (BCL7A, BCL7); Leukemia |
| oncology diseases and | (TAL1, TCL5, SCL, TAL2, FLT3, NBS1, NBS, ZNFN1A1, |
| disorders | IK1, LYF1, HOXD4, HOX4B, BCR, CML, PHL, ALL, ARNT, |
| | KRAS2, RASK2, GMPS, AF10, ARHGEF12, LARG, |
| | KIAA0382, CALM, CLTH, CEBPA, CEBP, CHIC2, BTL, |
| | FLT3, KIT, PBT, LPP, NPM1, NUP214, D9S46E, CAN, CAIN, |
| | RUNX1, CBFA2, AML1, WHSC1L1, NSD3, FLT3, AF1Q, |
| | NPM1, NUMA1, ZNF145, PLZF, PML, MYL, STAT5B, AF10, |
| | CALM, CLTH, ARL11, ARLTS1, P2RX7, P2X7, BCR, CML, |
| | PHL, ALL, GRAF, NF1, VRNF, WSS, NFNS, PTPN11, |
| | PTP2C, SHP2, NS1, BCL2, CCND1, PRAD1, BCL1, TCRA, |
| | GATA1, GF1, ERYF1, NFE1, ABL1, NQO1, DIA4, NMOR1, |
| | NUP214, D9S46E, CAN, CAIN) |
| Inflammation and immune | AIDS (KIR3DL1, NKAT3, NKB1, AMB11, KIR3DS1, IFNG, |
| related diseases and | CXCL12, SDF1); Autoimmune lymphoproliferative syndrome |
| disorders | (TNFRSF6, APT1, FAS, CD95, ALPS1A); Combined |
| | immunodeficiency, (IL2RG, SCIDX1, SCIDX, IMD4); HIV-1 |
| | (CCL5, SCYA5, D17S136E, TCP228), HIV susceptibility or |
| | infection (IL10, CSIF, CMKBR2, CCR2, CMKBR5, CCCKR5 |
| | (CCR5)); Immunodeficiencies (CD3E, CD3G, AICDA, AID, |
| | HIGM2, TNFRSF5, CD40, UNG, DGU, HIGM4, TNFSF5, |
| | CD40LG, HIGM1, IGM, FOXP3, IPEX, AIID, XPID, PIDX, |
| | TNFRSF14B, TACI); Inflammation (IL-10, IL-1 (IL-1a, IL-1b), |
| | IL-13, IL-17 (IL-17a (CTLA8), IL-17b, IL-17c, IL- 17d, IL- |
| | 17f), II-23, Cx3cr1, ptpn22, TNFa, NOD2/CARD15 for IBD, |
| | IL-6, IL-12 (IL-12a, IL-12b), CTLA4, Cx3cl1); Severe |
| | combined immunodeficiencies (SCIDs)(JAK3, JAKL, |
| | DCLREIC, ARTEMIS, SCIDA, RAG1, RAG2, ADA, PTPRC, |
| | CD45, LCA, IL7R, CD3D, T3D, IL2RG, SCIDX1, SCIDX, |
| | IMD4) |
| Metabolic, liver, kidney | Amyloid neuropathy (TTR, PALB); Amyloidosis (APOA1, |
| and protein diseases and | APP, AAA, CVAP, AD1, GSN, FGA, LYZ, TTR, PALB); |
| disorders | Cirrhosis (KRT18, KRT8, CIRH1A, NAIC, TEX292, |
| | KIAA1988); Cystic fibrosis (CFTR, ABCC7, CF, MRP7); |
| | Glycogen storage diseases (SLC2A2, GLUT2, G6PC, G6PT, |
| | G6PT1, GAA, LAMP2, LAMPB, AGL, GDE, GBE1, GYS2, |
| | PYGL, PFKM); Hepatic adenoma, 142330 (TCF1, HNF1A, |
| | MODY3), Hepatic failure, early onset, and neurologic disorder |
| | (SCOD1, SCO1), Hepatic lipase deficiency (LIPC), |
| | Hepatoblastoma, cancer and carcinomas (CTNNB1, PDGFRL, |
| | PDGRL, PRLTS, AXIN1, AXIN, CTNNB1, TP53, P53, LFS1, |
| | IGF2R, MPRI, MET, CASP8, MCH5; Medullary cystic kidney |
| | disease (UMOD, HNFJ, FJHN, MCKD2, ADMCKD2); |
| | Phenylketonuria (PAH, PKU1, QDPR, DHPR, PTS); Polycystic |
| | kidney and hepatic disease (FCYT, PKHD1, ARPKD, PKD1, |
| | PKD2, PKD4, PKDTS, PRKCSH, G19P1, PCLD, SEC63) |
| Muscular/Skeletal | Becker muscular dystrophy (DMD, BMD, MYF6), Duchenne |
| diseases and disorders | Muscular Dystrophy (DMD, BMD); Emery-Dreifuss muscular |
| | dystrophy (LMNA, LMN1, EMD2, FPLD, CMD1A, HGPS, |
| | LGMD1B, LMNA, LMN1, EMD2, FPLD, CMD1A); |
| | Facioscapulohumeral muscular dystrophy (FSHMD1A, |
| | FSHD1A); Muscular dystrophy (FKRP, MDC1C, LGMD2I, |
| | LAMA2, LAMM, LARGE, KIAA0609, MDC1D, FCMD, |
| | TTID, MYOT, CAPN3, CANP3, DYSF, LGMD2B, SGCG, |
| | LGMD2C, DMDA1, SCG3, SGCA, ADL, DAG2, LGMD2D, |
| | DMDA2, SGCB, LGMD2E, SGCD, SGD, LGMD2F, CMD1L, |
| | TCAP, LGMD2G, CMDIN, TRIM32, HT2A, LGMD2H, |
| | FKRP, MDC1C, LGMD2I, TTN, CMD1G, TMD, LGMD2J, |
| | POMT1, CAV3, LGMDIC, SEPN1, SELN, RSMD1, PLEC1, |
| | PLTN, EBS1); Osteopetrosis (LRP5, BMND1, LRP7, LR3, |
| | OPPG, VBCH2, CLCN7, CLC7, OPTA2, OSTMI, GL, |
| | TCIRG1, TIRC7, OC116, OPTB1); Muscular atrophy (VAPB, |
| | VAPC, ALS8, SMN1, SMA1, SMA2, SMA3, SMA4, BSCL2, |
| | SPG17, GARS, SMAD1, CMT2D, HEXB, IGHMBP2, |
| | SMUBP2, CATF1, SMARD1) |
| Dermatological diseases | Albinisim (TYR, OCA2, TYRP1, SLC45A2, LYST), |
| and disorders | Ectodermal dysplasias (EDAR, EDARADD, WNT10A), Ehlers- |
| | Danlos syndrome (COL5A1, COL5A2, COL1A1, COL1A2, |
| | COL3A1, TNXB, ADAMTS2, PLOD1, FKBP14), Ichthyosis- |
| | associated disorders (FLG, STS, TGM1, ALOXE3/ALOX12B, |
| | KRT1, KRT10, ABCA12, KRT2, GJB2, TGM1, ABCA12, |
| | CYP4F22, ALOXE3, CERS3, NSHDL, EBP, MBTPS2, GJB2, |
| | SPINK5, AGHD5, PHYH, PEX7, ALDH3A2, ERCC2, ERCC3, |
| | GFT2H5, GBA), Incontinentia pigmenti (IKBKG, NEMO), |
| | Tuberous sclerosis (TSC1, TSC2), Premature aging syndromes |
| | (POLR3A, PYCR1, LMNA, POLD1, WRN, DMPK) |
| Neurological and Neuronal | ALS (SOD1, ALS2, STEX, FUS, TARDBP, VEGF (VEGF-a, |
| diseases and disorders | VEGF-b, VEGF-c); Alzheimer disease (APP, AAA, CVAP, |
| | AD1, APOE, AD2, PSEN2, AD4, STM2, APBB2, FE65L1, |
| | NOS3, PLAU, URK, ACE, DCP1, ACE1, MPO, PACIP1, |
| | PAXIP1L, PTIP, A2M, BLMH, BMH, PSEN1, AD3); Autism |
| | (Mecp2, BZRAP1, MDGA2, Sema5A, Neurexin 1, GLO1, |
| | MECP2, RTT, PPMX, MRX16, MRX79, NLGN3, NLGN4, |
| | KIAA1260, AUTSX2); Fragile X Syndrome (FMR2, FXR1, |
| | FXR2, mGLUR5); Huntington's disease and disease like |
| | disorders (HD, IT15, PRNP, PRIP, JPH3, JP3, HDL2, TBP, |
| | SCA17); Parkinson disease (NR4A2, NURR1, NOT, TINUR, |
| | SNCAIP, TBP, SCA17, SNCA, NACP, PARK1, PARK4, DJ1, |
| | PARK7, LRRK2, PARK8, PINK1, PARK6, UCHL1, PARK5, |
| | SNCA, NACP, PARK1, PARK4, PRKN, PARK2, PDJ, DBH, |
| | NDUFV2); Rett syndrome (MECP2, RTT, PPMX, MRX16, |
| | MRX79, CDKL5, STK9, MECP2, RTT, PPMX, MRX16, |
| | MRX79, x-Synuclein, DJ-1); Schizophrenia (Neuregulin1 |
| | (Nrg1), Erb4 (receptor for Neuregulin), Complexin1 (Cplx1), |
| | Tph1 Tryptophan hydroxylase, Tph2, Tryptophan hydroxylase 2, |
| | Neurexin 1, GSK3, GSK3a, GSK3b, 5-HTT (Slc6a4), COMT, |
| | DRD (Drd1a), SLC6A3, DAOA, DTNBP1, Dao (Dao1)); |
| | Secretase Related Disorders (APH-1 (alpha and beta), Presenilin |
| | (Psen1), nicastrin, (Ncstn), PEN-2, Nos1, Parp1, Natl, Nat2); |
| | Trinucleotide Repeat Disorders (HTT (Huntington's Dx), |
| | SBMA/SMAX1/AR (Kennedy's Dx), FXN/X25 (Friedrich's |
| | Ataxia), ATX3 (Machado-Joseph's Dx), ATXN1 and ATXN2 |
| | (spinocerebellar ataxias), DMPK (myotonic dystrophy), |
| | Atrophin-1 and Atn1 (DRPLA Dx), CBP (Creb-BP - global |
| | instability), VLDLR (Alzheimer's), Atxn7, Atxn10) |
| Ocular diseases and | Age-related macular degeneration (Abcr, Ccl2, Cc2, cp |
| disorders | (ceruloplasmin), Timp3, cathepsinD, Vldlr, Ccr2); Cataract |
| | (CRYAA, CRYA1, CRYBB2, CRYB2, PITX3, BFSP2, CP49, |
| | CP47, CRYAA, CRYA1, PAX6, AN2, MGDA, CRYBA1, |
| | CRYB1, CRYGC, CRYG3, CCL, LIM2, MP19, CRYGD, |
| | CRYG4, BFSP2, CP49, CP47, HSF4, CTM, HSF4, CTM, MIP, |
| | AQP0, CRYAB, CRYA2, CTPP2, CRYBB1, CRYGD, CRYG4, |
| | CRYBB2, CRYB2, CRYGC, CRYG3, CCL, CRYAA, CRYA1, |
| | GJA8, CX50, CAE1, GJA3, CX46, CZP3, CAE3, CCM1, CAM, |
| | KRIT1); Corneal clouding and dystrophy (APOA1, TGFBI, |
| | CSD2, CDGG1, CSD, BIGH3, CDG2, TACSTD2, TROP2, |
| | M1S1, VSX1, RINX, PPCD, PPD, KTCN, COL8A2, FECD, |
| | PPCD2, PIP5K3, CFD); Cornea plana congenital (KERA, |
| | CNA2); Glaucoma (MYOC, TIGR, GLC1A, JOAG, GPOA, |
| | OPTN, GLCIE, FIP2, HYPL, NRP, CYP1B1, GLC3A, OPA1, |
| | NTG, NPG, CYP1B1, GLC3A); Leber congenital |
| | amaurosis (CRB1, RP12, CRX, CORD2, CRD, RPGRIP1, |
| | LCA6, CORD9, RPE65, RP20, AIPL1, LCA4, GUCY2D, |
| | GUC2D, LCA1, CORD6, RDH12, LCA3); Macular dystrophy |
| | (ELOVL4, ADMD, STGD2, STGD3, RDS, RP7, PRPH2, |
| | PRPH, AVMD, AOFMD, VMD2) |

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of and any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

It is understood that where a numerical range is recited herein, the present invention contemplates each integer between, and including, the upper and lower limits, unless otherwise stated.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

The terms “polynucleotide”, “nucleotide”, “nucleotide sequence”, “nucleic acid” and “oligonucleotide” are used interchangeably. They refer to a polymeric form of nucleotides of any length, either deoxyribonueleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, in Irons, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers, A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “nucleotide analog” or “modified nucleotide” refers to a nucleotide that contains one or more chemical modifications (e.g., substitutions), in or on the nitrogenous base of the nucleoside (e.g., cytosine (C), thymine (T) or uracil (U), adenine (A) or guanine (G)), in or on the sugar moiety of the nucleoside (e.g., ribose, deoxyribose, modified ribose, modified deoxyribose, six-membered sugar analog, or open-chain sugar analog), or the phosphate. Each of the RNA sequences described herein may comprise one or more nucleotide analogs.

As used herein, the following nucleotide identifiers are used to represent a referenced nucleotide base(s):

| | |
|---|---|
| Nucleotide | |
| reference | Base(s) represented |

| | | | | |
|---|---|---|---|---|
| A | A | | | |
| C | | C | | |
| G | | | G | |
| T | | | | T |
| W | A | | | T |
| S | | C | G | |
| M | A | C | | |
| K | | | G | T |
| R | A | | G | |
| Y | | C | | T |
| B | | C | G | T |
| D | A | | G | T |
| H | A | C | | T |
| V | A | C | G | |
| N | A | C | G | T |

As used herein, the term “targeting sequence” or “targeting molecule” refers a nucleotide sequence or molecule comprising a nucleotide sequence that is capable of hybridizing to a specific target sequence, e.g., the targeting sequence has a nucleotide sequence which is at least partially complementary to the sequence being targeted along the length of the targeting sequence. The targeting sequence or targeting molecule may be part of a targeting RNA molecule that can form a complex with a CRISPR nuclease, e.g. via a scaffold portion, with the targeting sequence serving as the targeting portion, e.g. spacer portion, of the CRISPR complex. When the RNA molecule having the targeting sequence is present contemporaneously with the CRISPR nuclease, the RNA molecule, alone or in combination with an additional one or more RNA molecules (e.g. a tracrRNA molecule), is capable of targeting the CRISPR nuclease to the specific target sequence. As non-limiting example, a guide sequence portion of a CRISPR RNA molecule or single-guide RNA molecule may serve as a targeting molecule. Each possibility represents a separate embodiment. A targeting sequence can be custom designed to target any desired sequence.

The term “targets” as used herein, refers to preferential hybridization of a targeting sequence or a targeting molecule to a nucleic acid having a targeted nucleotide sequence. It is understood that the term “targets” encompasses variable hybridization efficiencies, such that there is preferential targeting of the nucleic acid having the targeted nucleotide sequence, but unintentional off-target hybridization in addition to on-target hybridization might also occur. It is understood that where an RNA molecule targets a sequence, a complex of the RNA molecule and a CRISPR nuclease molecule targets the sequence for nuclease activity.

The “guide sequence portion” of an RNA molecule refers to a nucleotide sequence that is capable of hybridizing to a specific target DNA sequence, e.g., the guide sequence portion has a nucleotide sequence which is partially or fully complementary to the DNA sequence being targeted along the length of the guide sequence portion. In some embodiments, the guide sequence portion is 17, 18, 19, 20, 21, 22, 23, 24, 25, 26, 27, 28, 29, 30, 31, 32, 33, 34, 35, 36, 37, 38, 39, 41, 42, 43, 44, 45, 46, 47, 48, 49 or 50 nucleotides in length, or approximately 17-50, 17-49, 17-48, 17-47, 17-46, 17-45, 17-44, 17-43, 17-42, 17-41, 17-40, 17-39, 17-38, 17-37, 17-36, 17-35, 17-34, 17-33, 17-31, 17-30, 17-29, 17-28, 17-27, 17-26, 17-25, 17-24, 17-22, 17-21, 18-25, 18-24, 18-23, 18-22, 18-21, 19-25, 19-24, 19-23, 19-22, 19-21, 19-20, 20-22, 18-20, 20-21, 21-22, or 17-nucleotides in length. Preferably, the entire length of the guide sequence portion is fully complementary to the DNA sequence being targeted along the length of the guide sequence portion. The guide sequence portion may be part of an RNA molecule that can form a complex with a CRISPR nuclease with the guide sequence portion serving as the DNA targeting portion of the CRISPR complex. When the RNA molecule having the guide sequence portion is present contemporaneously with the CRISPR molecule, alone or in combination with an additional one or more RNA molecules (e.g. a tracrRNA molecule), the RNA molecule is capable of targeting the CRISPR nuclease to the specific target DNA sequence. Accordingly, a CRISPR complex can be formed by direct binding of the RNA molecule having the guide sequence portion to a CRISPR nuclease or by binding of the RNA molecule having the guide sequence portion and an additional one or more RNA molecules to the CRISPR nuclease. Each possibility represents a separate embodiment. A guide sequence portion can be custom designed to target any desired sequence. Accordingly, a molecule comprising a “guide sequence portion” is a type of targeting molecule. Throughout this application, the terms “guide molecule,” “RNA guide molecule,” “guide RNA molecule,” and “gRNA molecule” are synonymous with a molecule comprising a guide sequence portion.

In the context of targeting a DNA sequence that is present in a plurality of cells, it is understood that the targeting encompasses hybridization of the guide sequence portion of the RNA molecule with the sequence in one or more of the cells, and also encompasses hybridization of the RNA molecule with the target sequence in fewer than all of the cells in the plurality of cells. Accordingly, it is understood that where an RNA molecule targets a sequence in a plurality of cells, a complex of the RNA molecule and a CRISPR nuclease is understood to hybridize with the target sequence in one or more of the cells, and also may hybridize with the target sequence in fewer than all of the cells. Accordingly, it is understood that the complex of the RNA molecule and the CRISPR nuclease introduces a double strand break in relation to hybridization with the target sequence in one or more cells and may also introduce a double strand break in relation to hybridization with the target sequence in fewer than all of the cells. As used herein, the term “modified cells” refers to cells in which a double strand break is affected by a complex of an RNA molecule and the CRISPR nuclease as a result of hybridization with the target sequence, i.e. on-target hybridization.

As used herein the term “wild type” is a term of the art understood by skilled persons and means the typical form of an organism, strain, gene or characteristic as it occurs in nature as distinguished from mutant or variant forms. Accordingly, as used herein, where a sequence of amino acids or nucleotides refers to a wild type sequence, a variant refers to variant of that sequence, e.g., comprising substitutions, deletions, insertions. In embodiments of the present invention, an engineered CRISPR nuclease is a variant CRISPR nuclease comprising at least one amino acid modification (e.g., substitution, deletion, and/or insertion) compared to the OMNI-50 CRISPR nuclease indicated in Table 1.

The terms “non-naturally occurring” or “engineered” are used interchangeably and indicate human manipulation. The terms, when referring to nucleic acid molecules or polypeptides may mean that the nucleic acid molecule or the polypeptide is at least substantially free from at least one other component with which they are naturally associated in nature and as found in nature.

As used herein the term “amino acid” includes natural and/or unnatural or synthetic amino acids, including glycine and both the D or I, optical isomers, and amino acid analogs and peptidomimetics.

As used herein, “genomic DNA” refers to linear and/or chromosomal DNA and/or to plasmid or other extrachromosomal DNA sequences present in the cell or cells of interest. In some embodiments, the cell of interest is a eukaryotic cell. In some embodiments, the cell of interest is a prokaryotic cell. In some embodiments, the methods produce double-stranded breaks (DSBs) at pre-determined target sites in a genomic DNA sequence, resulting in mutation, insertion, and/or deletion of DNA sequences at the target site(s) in a genome.

“Eukaryotic” cells include, but are not limited to, fungal cells (such as yeast), plant cells, animal cells, mammalian cells and human cells.

The term “nuclease” as used herein refers to an enzyme capable of cleaving the phosphodiester bonds between the nucleotide subunits of nucleic acid. A nuclease may be isolated or derived from a natural source. The natural source may be any living organism. Alternatively, a nuclease may be a modified or a synthetic protein which retains the phosphodiester bond cleaving activity.

The term “PAM” as used herein refers to a nucleotide sequence of a target DNA located in proximity to the targeted DNA sequence and recognized by the CRISPR nuclease. The PAM sequence may differ depending on the nuclease identity.

The term “mutation disorder” or “mutation disease” as used herein refers to any disorder or disease that is related to dysfunction of a gene caused by a mutation. A dysfunctional gene manifesting as a mutation disorder contains a mutation in at least one of its alleles and is referred to as a “disease-associated gene.” The mutation may be in any portion of the disease-associated gene, for example, in a regulatory, coding, or non-coding portion. The mutation may be any class of mutation, such as a substitution, insertion, or deletion. The mutation of the disease-associated gene may manifest as a disorder or disease according to the mechanism of any type of mutation, such as a recessive, dominant negative, gain-of-function, loss-of-function, or a mutation leading to haploinsufficiency of a gene product.

A skilled artisan will appreciate that embodiments of the present invention disclose RNA molecules comprising a scaffold portion capable of complexing with an OMNI-50 CRISPR nuclease and activating the OMNI-50 CRISPR nuclease to be targeted to a target DNA site of interest that is adjacent to a protospacer adjacent motif (PAM). The OMNI-50 CRISPR nuclease is targeted to a DNA site of interest by a guide sequence portion (i.e. a RNA spacer) having complementarity to the target DNA site of interest. The nuclease then mediates cleavage of target DNA to create a double-strand break within the protospacer target site.

The term “protein binding sequence” or “nuclease binding sequence” refers to a sequence capable of binding with a CRISPR nuclease to form a CRISPR complex. A skilled artisan will understand that scaffold RNA or a tracrRNA capable of binding with a CRISPR nuclease to form a CRISPR complex comprises a protein or nuclease binding sequence.

An “RNA binding portion” of a CRISPR nuclease refers to a portion of the CRISPR nuclease which may bind to an RNA molecule to form a CRISPR complex, e.g. the nuclease binding sequence of an RNA scaffold portion of a sgRNA. An “activity portion” or “active portion” of a CRISPR nuclease refers to a portion of the CRISPR nuclease which effects a double strand break in a DNA molecule, for example when in complex with a DNA-targeting RNA molecule.

The term “RNA scaffold” or “scaffold” refers to a portion of a non-naturally occurring molecule that comprises a crRNA portion covalently linked to a tracrRNA portion. As used herein, a “crRNA portion” comprises a crRNA repeat sequence. As used herein, a “tracrRNA portion” comprises a tracrRNA anti-repeat sequence. A tracrRNA portion may further comprise additional tracrRNA sequences linked to the tracrRNA anti-repeat sequence. Such sequences may include, but are not limited to, a nexus, hairpin, or other tracrRNA sequences upstream or downstream of a nexus, hairpin, or tracrRNA anti-repeat sequence. Accordingly, a tracrRNA portion of an RNA scaffold comprises an anti-repeat sequence, which is optionally linked to additional tracrRNA sections.

As used herein, an RNA molecule comprising an RNA scaffold portion and an RNA guide sequence portion (or RNA spacer portion) serves as a single-guide RNA (sgRNA) molecule. The RNA scaffold portion of the sgRNA specifically binds and activates an CRISPR nuclease, and the RNA spacer portion of the sgRNA targets CRISPR nuclease to a DNA target site. For example, a sgRNA molecule may be formed by covalent linkage of a guide sequence portion to a crRNA repeat sequence portion of an RNA scaffold.

Accordingly, in embodiments of the present invention, the RNA molecule may be designed as a synthetic fusion of a scaffold portion and a spacer portion, together forming a single guide RNA (sgRNA) capable of binding and targeting an OMNI-50 CRISPR nuclease. See Jinek et al., Science (2012).

Embodiments of the present invention may also form CRISPR complexes utilizing a separate crRNA molecule and a separate tracrRNA molecule. In such embodiments the crRNA molecule may hybridize with the tracrRNA molecule via at least partial hybridization between a crRNA repeat sequence portion of the crRNA molecule and a tracrRNA anti-repeat sequence portion of the tracrRNA molecule. Such partial hybridization may also contain a typical bulge that separates the hybridized RNA nucleotides into an “upper” and “lower” stem. Separate crRNA and tracrRNA molecules may be advantageous in certain applications of the invention described herein.

In embodiments of the present invention a scaffold portion of an RNA molecule may comprise a “nexus” region and/or “hairpin” regions which may further define the structure of the RNA molecule. (See Briner et al., Molecular Cell (2014)).

As used herein, the term “direct repeat sequence” refers to two or more repeats of a specific amino acid sequence or nucleotide sequence.

As used herein, an RNA sequence or molecule capable of “interacting with” or “binding” with a CRISPR nuclease refers to the RNA sequence or molecules ability to form a CRISPR complex with the CRISPR nuclease.

As used herein, the term “operably linked” refers to a relationship (i.e. fusion, hybridization) between two sequences or molecules permitting them to function in their intended manner. In embodiments of the present invention, when an RNA molecule is operably linked to a promoter, both the RNA molecule and the promotor are permitted to function in their intended manner.

As used herein, the term “heterologous promoter” refers to a promoter that does not naturally occur together with the molecule or pathway being promoted.

As used herein, a sequence or molecule has an X% “sequence identity” to another sequence or molecule if X% of nucleotides or amino acids between the sequences of molecules are the same and in the same relative position. For example, a first nucleotide sequence having at least a 95% sequence identity with a second nucleotide sequence will have at least 95% of nucleotides, in the same relative position, identical with the other sequence. As non-limiting example, sequence identity may be determined by creating an alignment of a first nucleotide sequence to a second nucleotide sequence, for example, by applying the Needleman—Wunsch algorithm.

### Delivery

The CRISPR nuclease or CRISPR compositions described herein may include and be delivered as a protein, DNA molecules, RNA molecules, Ribonucleoproteins (RNP), nucleic acid vectors, or any combination thereof. In some embodiments, the RNA molecule comprises a chemical modification. Non-limiting examples of suitable chemical modifications include 2′-0-methyl (M), 2′-0-methyl, 3′phosphorothioate (MS) or 2′-0-methyl, 3′thioPACE (MSP), pseudouridine, and 1-methyl pseudo-uridine. Each possibility represents a separate embodiment of the present invention.

The CRISPR nucleases and/or polynucleotides encoding same described herein, and optionally additional proteins (e.g., ZFPs, TALENs, transcription factors, restriction enzymes) and/or nucleotide molecules such as guide RNA may be delivered to a target cell by any suitable means. The target cell may be any type of cell e.g., eukaryotic or prokaryotic, in any environment e.g., isolated or not, maintained in culture, in vitro, ex vivo, in vivo or in planta.

In some embodiments, the composition to be delivered includes mRNA of the nuclease and RNA of the guide molecule. In some embodiments, the composition to be delivered includes mRNA of the nuclease, RNA of the guide and a donor template. In some embodiments, the composition to be delivered includes the CRISPR nuclease and guide RNA. In some embodiments, the composition to be delivered includes the CRISPR nuclease, guide RNA and a donor template for gene editing via, for example, homology directed repair. In some embodiments, the composition to be delivered includes mRNA of the nuclease, DNA-targeting RNA and the tracrRNA. In some embodiments, the composition to be delivered includes mRNA of the nuclease, DNA-targeting RNA and the tracrRNA and a donor template. In some embodiments, the composition to be delivered includes the CRISPR nuclease DNA-targeting RNA and the tracrRNA. In some embodiments, the composition to be delivered includes the CRISPR nuclease, DNA-targeting RNA and the tracrRNA and a donor template for gene editing via, for example, homology directed repair.

Any suitable viral vector system may be used to deliver RNA compositions. Conventional viral and non-viral based gene transfer methods can be used to introduce nucleic acids and/or CRISPR nuclease in cells (e.g., mammalian cells, plant cells, etc.) and target tissues. Such methods can also be used to administer nucleic acids encoding and/or CRISPR nuclease protein to cells in vitro. In certain embodiments, nucleic acids and/or CRISPR nuclease are administered for in vivo or ex vivo gene therapy uses. Non-viral vector delivery systems include naked nucleic acid, and nucleic acid complexed with a delivery vehicle such as a liposome or poloxamer. For a review of gene therapy procedures, see Anderson, Science (1992); Nabel and Felgner, TIBTECH (1993); Mitani and Caskey, TIBTECH (1993); Dillon, TIBTECH (1993); Miller, Nature (1992); Van Brunt, Biotechnology (1988); Vigne et al., Restorative Neurology and Neuroscience 8:35-36 (1995); Kremer and Perricaudet, British Medical Bulletin (1995); Haddada et al., Current Topics in Microbiology and Immunology (1995); and Yu et al., Gene Therapy 1:13-26 (1994).

Methods of non-viral delivery of nucleic acids and/or proteins include electroporation, lipofection, microinjection, biolistics, particle gun acceleration, virosomes, liposomes, immunoliposomes, polycation or lipid:nucleic acid conjugates, artificial virions, and agent-enhanced uptake of nucleic acids or can be delivered to plant cells by bacteria or viruses (e.g., Agrobacterium, Rhizobium sp. NGR234, Sinorhizoboiummeliloti, Mesorhizobium loti, tobacco mosaic virus, potato virus X, cauliflower mosaic virus and cassava vein mosaic virus. See, e.g., Chung et al. Trends Plant Sci. (2006). Sonoporation using, e.g., the Sonitron 2000 system (Rich-Mar) can also be used for delivery of nucleic acids. Cationic-lipid mediated delivery of proteins and/or nucleic acids is also contemplated as an in vivo or in vitro delivery method. See Zuris et al., Nat. Biotechnol. (2015), Coelho et al., N. Engl. J. Med. (2013); Judge et al., Mol. Ther. (2006); and Basha et al., Mol. Ther. (2011).

Additional exemplary nucleic acid delivery systems include those provided by Amaxa® Biosystems (Cologne, Germany), Maxcyte, Inc. (Rockville, Md.), BTX Molecular Delivery Systems (Holliston, Mass.) and Copernicus Therapeutics Inc., (see for example U.S. Pat. No. 6,008,336). Lipofection is described in e.g., U.S. Pat. Nos. 5,049,386, 4,946,787; and 4,897,355) and lipofection reagents are sold commercially (e.g., Transfectam™, Lipofectin™ and Lipofectamine™ RNAiMAX). Cationic and neutral lipids that are suitable for efficient receptor-recognition lipofection of polynucleotides include those disclosed in PCT International Publication Nos. WO/1991/017424 and WO/1991/016024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science (1995); Blaese et al., Cancer Gene Ther. (1995); Behr et al., Bioconjugate Chem. (1994); Remy et al., Bioconjugate Chem. (1994); Gao and Huang, Gene Therapy (1995); Ahmad and Allen, Cancer Res., (1992); U.S. Pat. Nos. 4,186,183; 4,217,344; 4,235,871; 4,261,975; 4,485,054; 4,501,728; 4,774,085; 4,837,028; and 4,946,787).

Additional methods of delivery include the use of packaging the nucleic acids to be delivered into EnGenelC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (see MacDiamid et al., Nature Biotechnology (2009)).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, recombinant retroviral, lentivirus, adenoviral, adeno-associated, vaccinia and herpes simplex virus vectors for gene transfer. However, an RNA virus is preferred for delivery of the RNA compositions described herein. Additionally, high transduction efficiencies have been observed in many different cell types and target tissues. Nucleic acid of the invention may be delivered by non-integrating lentivirus. Optionally, RNA delivery with Lentivirus is utilized. Optionally the lentivirus includes mRNA of the nuclease, RNA of the guide. Optionally the lentivirus includes mRNA of the nuclease, RNA of the guide and a donor template. Optionally, the lentivirus includes the nuclease protein, guide RNA. Optionally, the lentivirus includes the nuclease protein, guide RNA and/or a donor template for gene editing via, for example, homology directed repair. Optionally the lentivirus includes mRNA of the nuclease, DNA-targeting RNA, and the tracrRNA. Optionally the lentivirus includes mRNA of the nuclease, DNA-targeting RNA, and the tracrRNA, and a donor template. Optionally, the lentivirus includes the nuclease protein, DNA-targeting RNA, and the tracrRNA. Optionally, the lentivirus includes the nuclease protein, DNA-targeting RNA, and the tracrRNA, and a donor template for gene editing via, for example, homology directed repair.

As mentioned above, the compositions described herein may be delivered to a target cell using a non-integrating lentiviral particle method, e.g. a LentiFlash® system. Such a method may be used to deliver mRNA or other types of RNAs into the target cell, such that delivery of the RNAs to the target cell results in assembly of the compositions described herein inside of the target cell. See also PCT International Publication Nos. WO2013/014537, WO2014/016690, WO2016185125, WO2017194902, and WO2017194903.

The tropism of a retrovirus can be altered by incorporating foreign envelope proteins, expanding the potential target population of target cells. Lentiviral vectors are retroviral vectors capable of transducing or infecting non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (see, e.g., Buchscher Panganiban, J. Virol. (1992); Johann et al., J. Virol. (1992); Sommerfelt et al., Virol. (1990); Wilson et al., J. Virol. (1989); Miller et al., J. Virol. (1991); PCT International Publication No. WO/1994/026877A1).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (Dunbar et al., Blood (1995); Kohn et al., Nat. Med. (1995); Malech et al., PNAS (1997)). PA317/pLASN was the first therapeutic vector used in a gene therapy trial. (Blaese et al., Science (1995)). Transduction efficiencies of 50% or greater have been observed for MFG-S packaged vectors. (Ellem et al., Immunol Immunother. (1997); Dranoff et al., Hum. Gene Ther. (1997).

Packaging cells are used to form virus particles that are capable of infecting a host cell. Such cells include 293 cells, which package adenovirus, AAV, and psi.2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additionally, AAV can be produced at clinical scale using baculovirus systems (see U.S. Pat. No. 7,479,554).

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al., Proc. Natl. Acad. Sci. USA (1995), reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to non-viral vectors. Such vectors can be engineered to contain specific uptake sequences which favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector. In some embodiments, delivery of mRNA in vivo and ex vivo, and RNPs delivery may be utilized.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with an RNA composition, and re-infused back into the subject organism (e.g., patient). Various cell types suitable for ex vivo transfection are well known to those of skill in the art (see, e.g., Freshney, “Culture of Animal Cells, A Manual of Basic Technique and Specialized Applications (6th edition, 2010)) and the references cited therein for a discussion of how to isolate and culture cells from patients).

Suitable cells include but not limited to eukaryotic and prokaryotic cells and/or cell lines. Non-limiting examples of such cells or cell lines generated from such cells include COS, CHO (e.g., CHO-S, CHO-K1, CHO-DG44, CHO-DUXB11, CHO-DUKX, CHOK1SV), VERO, MDCK, WI38, V79, B14AF28-G3, BHK, HaK, NSO, SP2/0-Ag14, HeLa, HEK293 (e.g., HEK293-F, HEK293-H, HEK293-T), and perC6 cells, any plant cell (differentiated or undifferentiated) as well as insect cells such as Spodopterafugiperda (Sf), or fungal cells such as Saccharomyces, Pichia and Schizosaccharomyces. In certain embodiments, the cell line is a CHO-K1, MDCK or HEK293 cell line. Additionally, primary cells may be isolated and used ex vivo for reintroduction into the subject to be treated following treatment with the nucleases (e.g. ZFNs or TALENs) or nuclease systems (e.g. CRISPR). Suitable primary cells include peripheral blood mononuclear cells (PBMC), and other blood cell subsets such as, but not limited to, CD4+ T cells or CD8+ T cells. Suitable cells also include stem cells such as, by way of example, embryonic stem cells, induced pluripotent stem cells, hematopoietic stem cells (CD34+), neuronal stem cells and mesenchymal stem cells.

In one embodiment, stem cells are used in ex vivo procedures for cell transfection and gene therapy. The advantage to using stem cells is that they can be differentiated into other cell types in-vitro or can be introduced into a mammal (such as the donor of the cells) where they will engraft in the bone marrow. Methods for differentiating CD34+ cells in vitro into clinically important immune cell types using cytokines such a GM-C SF, IFN-gamma. and TNF-alpha are known (as a non-limiting example see, Inaba et al., J. Exp. Med. (1992)).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and Tad (differentiated antigen presenting cells) (as a non-limiting example see Inaba et al., J. Exp. Med. (1992)). Stem cells that have been modified may also be used in some embodiments.

Notably, the compositions described herein may be suitable for genome editing in post-mitotic cells or any cell which is not actively dividing, e.g., arrested cells. Examples of post-mitotic cells which may be edited using a CRISPR nuclease of the present invention include, but are not limited to, myocyte, a cardiomyocyte, a hepatocyte, an osteocyte and a neuron.

Vectors (e.g., retroviruses, liposomes, etc.) containing therapeutic RNA compositions can also be administered directly to an organism for transduction of cells in vivo. Alternatively, naked RNA or mRNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Vectors suitable for introduction of transgenes into immune cells (e.g., T-cells) include non-integrating lentivirus vectors. See, for example, U.S. Patent Publication No. 2009/0117617.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

### DNA Repair by Homologous Recombination

The term “homology-directed repair” or “HDR” refers to a mechanism for repairing DNA damage in cells, for example, during repair of double-stranded and single-stranded breaks in DNA. HDR requires nucleotide sequence homology and uses a “nucleic acid template” (nucleic acid template or donor template used interchangeably herein) to repair the sequence where the double-stranded or single break occurred (e.g., DNA target sequence). This results in the transfer of genetic information from, for example, the nucleic acid template to the DNA target sequence. HDR may result in alteration of the DNA target sequence (e.g., insertion, deletion, mutation) if the nucleic acid template sequence differs from the DNA target sequence and part or all of the nucleic acid template polynucleotide or oligonucleotide is incorporated into the DNA target sequence. In some embodiments, an entire nucleic acid template polynucleotide, a portion of the nucleic acid template polynucleotide, or a copy of the nucleic acid template is integrated at the site of the DNA target sequence.

The terms “nucleic acid template” and “donor”, refer to a nucleotide sequence that is inserted or copied into a genome. The nucleic acid template comprises a nucleotide sequence, e.g., of one or more nucleotides, that will be added to or will template a change in the target nucleic acid or may be used to modify the target sequence. A nucleic acid template sequence may be of any length, for example between 2 and 10,000 nucleotides in length (or any integer value there between or there above), preferably between about 100 and 1,000 nucleotides in length (or any integer there between), more preferably between about 200 and 500 nucleotides in length. A nucleic acid template may be a single stranded nucleic acid, a double stranded nucleic acid. In some embodiment, the nucleic acid template comprises a nucleotide sequence, e.g., of one or more nucleotides, that corresponds to wild type sequence of the target nucleic acid, e.g., of the target position. In some embodiment, the nucleic acid template comprises a ribonucleotide sequence, e.g., of one or more ribonucleotides, that corresponds to wild type sequence of the target nucleic acid, e.g., of the target position. In some embodiment, the nucleic acid template comprises modified ribonucleotides.

Insertion of an exogenous sequence (also called a “donor sequence,” donor template” or “donor”), for example, for correction of a mutant gene or for increased expression of a wild-type gene can also be carried out. It will be readily apparent that the donor sequence is typically not identical to the genomic sequence where it is placed. A donor sequence can contain a non-homologous sequence flanked by two regions of homology to allow for efficient HDR at the location of interest. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

The donor polynucleotide can be DNA or RNA, single-stranded and/or double-stranded and can be introduced into a cell in linear or circular form. See, e.g., U.S. Patent Publication Nos. 2010/0047805; 2011/0281361; 2011/0207221; and 2019/0330620. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang and Wilson, Proc. Natl. Acad. Sci. USA (1987); Nehls et al., Science (1996). Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

Accordingly, embodiments of the present invention using a donor template for repair may use a DNA or RNA, single-stranded and/or double-stranded donor template that can be introduced into a cell in linear or circular form. In embodiments of the present invention a gene-editing composition comprises: (1) an RNA molecule comprising a guide sequence to affect a double-strand break in a gene prior to repair and (2) a donor RNA template for repair, the RNA molecule comprising the guide sequence is a first RNA molecule and the donor RNA template is a second RNA molecule. In some embodiments, the guide RNA molecule and template RNA molecule are connected as part of a single molecule.

A donor sequence may also be an oligonucleotide and be used for gene correction or targeted alteration of an endogenous sequence. The oligonucleotide may be introduced to the cell on a vector, may be electroporated into the cell, or may be introduced via other methods known in the art. The oligonucleotide can be used to ‘correct’ a mutated sequence in an endogenous gene (e.g., the sickle mutation in beta globin), or may be used to insert sequences with a desired purpose into an endogenous locus.

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome or poloxamer, or can be delivered by recombinant viruses (e.g., adenovirus, AAV, herpesvirus, retrovirus, lentivirus and integrase defective lentivirus (IDLV)).

The donor is generally inserted so that its expression is driven by the endogenous promoter at the integration site, namely the promoter that drives expression of the endogenous gene into which the donor is inserted. However, it will be apparent that the donor may comprise a promoter and/or enhancer, for example a constitutive promoter or an inducible or tissue specific promoter.

The donor molecule may be inserted into an endogenous gene such that all, some or none of the endogenous gene is expressed. For example, a transgene as described herein may be inserted into an endogenous locus such that some (N-terminal and/or C-terminal to the transgene) or none of the endogenous sequences are expressed, for example as a fusion with the transgene. In other embodiments, the transgene (e.g., with or without additional coding sequences such as for the endogenous gene) is integrated into any endogenous locus, for example a safe-harbor locus, for example a CCR5 gene, a CXCR4 gene, a PPP1R12c (also known as AAVS1) gene, an albumin gene or a Rosa gene. See, e.g., U.S. Pat. Nos. 7,951,925 and 8,110,379; U.S. Publication Nos. 2008/0159996; 20100/0218264; 2010/0291048; 2012/0017290; 2011/0265198; 2013/0137104; 2013/0122591; 2013/0177983 and 2013/0177960 and U.S. Provisional Application No. 61/823,689).

When endogenous sequences (endogenous or part of the transgene) are expressed with the transgene, the endogenous sequences may be full-length sequences (wild-type or mutant) or partial sequences. Preferably the endogenous sequences are functional. Non-limiting examples of the function of these full length or partial sequences include increasing the serum half-life of the polypeptide expressed by the transgene (e.g., therapeutic gene) and/or acting as a carrier.

Furthermore, although not required for expression, exogenous sequences may also include transcriptional or translational regulatory sequences, for example, promoters, enhancers, insulators, internal ribosome entry sites, sequences encoding 2A peptides and/or polyadenylation signals.

In certain embodiments, the donor molecule comprises a sequence selected from the group consisting of a gene encoding a protein (e.g., a coding sequence encoding a protein that is lacking in the cell or in the individual or an alternate version of a gene encoding a protein), a regulatory sequence and/or a sequence that encodes a structural nucleic acid such as a microRNA or siRNA.

For the foregoing embodiments, each embodiment disclosed herein is contemplated as being applicable to each of the other disclosed embodiment. For example, it is understood that any of the RNA molecules or compositions of the present invention may be utilized in any of the methods of the present invention.

As used herein, all headings are simply for organization and are not intended to limit the disclosure in any manner. The content of any individual section may be equally applicable to all sections.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Generally, the nomenclature used herein, and the laboratory procedures utilized in the present invention include molecular, biochemical, microbiological and recombinant DNA techniques. Such techniques are thoroughly explained in the literature. See, for example, Sambrook et al., “Molecular Cloning: A laboratory Manual” (1989); Ausubel, R. M. (Ed.), “Current Protocols in Molecular Biology” Volumes I-III (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (Eds.), “Genome Analysis: A Laboratory Manual Series”, Vols. 1-4, Cold Spring Harbor Laboratory Press, New York (1998); Methodologies as set forth in U.S. Pat. Nos. 4,666,828; 4,683,202; 4,801,531; 5,192,659 and 5,272,057; Cellis, J. E. (Ed.), “Cell Biology: A Laboratory Handbook”, Volumes I-III (1994); Freshney, “Culture of Animal Cells - A Manual of Basic Technique” Third Edition, Wiley-Liss, N. Y. (1994); Coligan J. E. (Ed.), “Current Protocols in Immunology” Volumes I-III (1994); Stites et al. (Eds.), “Basic and Clinical Immunology” (8th Edition), Appleton & Lange, Norwalk, CT (1994); Mishell and Shiigi (Eds.), “Strategies for Protein Purification and Characterization—A Laboratory Course Manual” CSHL Press (1996); Clokie and Kropinski (Eds.), “Bacteriophage Methods and Protocols”, Volume 1: Isolation, Characterization, and Interactions (2009), all of which are incorporated by reference. Other general references are provided throughout this document.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### Experimental Details

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### Methods

OMNI-50 sequences, such as the OMNI-50 CRISPR repeat (crRNA) sequence, OMNI-50 transactivating crRNA (tracrRNA) sequence, and OMNI-50 nuclease polypeptide sequence, were predicted from Ezakiella peruensis strain M6.X2.

The full-length scaffold with spacer optimization, the OMNI-50 “NGG” PAM identification, and the activity in mammalian cells were disclosed in PCT International Application No. PCT/US2020/030782, the entire contents of which is hereby incorporated by reference. An overview of OMNI-50 elements can be found below in Table 1.

### OMNI-50 Protein Expression

The expression methods for protein production and synthetic guide production for RNP assembly was described in PCT International Application No. PCT/US2020/030782. Briefly, The nuclease open reading frame was codon optimized for human cells (Table 1) and cloned into a modified pET9a plasmid with the following elements: SV40 NLS-OMNI-50 ORF (from 2^{nd} amino acid, human optimized)-HA tag-SV40 NLS-8 His-tag. The sequence can be found in Table 2. The OMNI-50 construct was expressed in T7-express cells (NEB). Cells were grown in TB, and expressed in mid-log phase by addition of 1mM IPTG while lowering the temperature of the culture to 18° C. Cells were lysed using sonication and the cleared lysate was purified using a Ni-NTA column. Fractions containing OMNI protein were collected and further purified by SEC on HiLoad 16/600 Superdex 200 pg-SEC, AKTA Pure (GE Healthcare Life Sciences). Fractions containing OMNI protein were pooled and concentrated to 10 mg/ml stocks and flash-frozen in liquid nitrogen and stored at −80° C.

Synthetic sgRNA Used

All synthetic sgRNAs of OMNI-50 were synthesized with three 2′-O-methyl 3′-phosphorothioate at the 3′ and 5′ ends (Agilent or Synthego).

RNP Activity In-Vitro

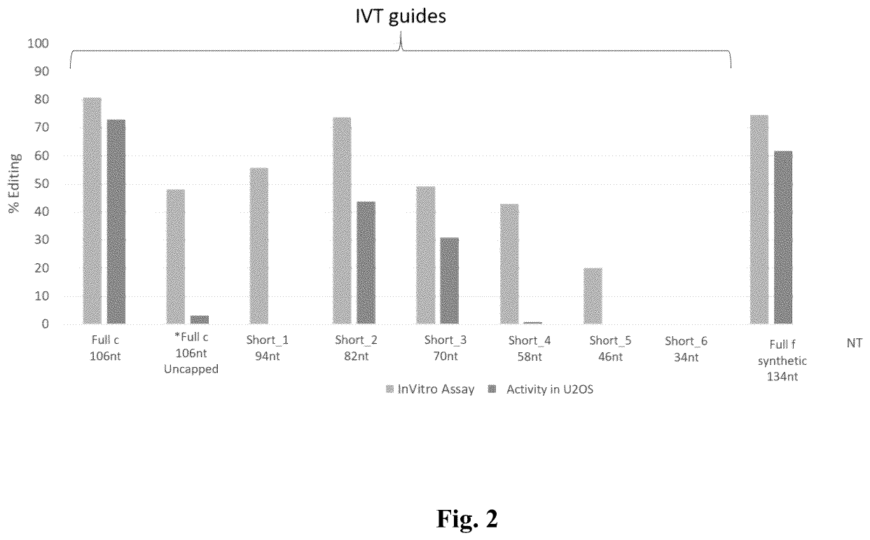

RNPs were formed by incubating 1 mg/mL protein with 1 μM IVT-transcribed or synthetically-manufactured sgRNAs at room-temperature for 10 mins. OMNI-50 RNP was reacted in the recommended cleavage buffer with 10Ong of linear 5′-FAM-labeled DNA substrates containing a g35 protospacer that is targeted by the sgRNA adjacent to the OMNI's PAM sequence (Table 8). Cleavage efficiency was calculated by the fluorescence intensity of the cut fragment divided by the sum of the fluorescence intensities of the cut and uncut fragments (FIG. 2).

### Activity in Mammalian Cell Lines

The different spacers and targets of the scaffolds assayed here are listed in Tables 8. RNPs were assembled by mixing 100 uM nuclease with 120 uM of synthetic guide and 100 uM Cas9 electroporation enhancer (IDT). After 10 mins of incubation at room-temperature, the RNP complexes were mixed with 200,000 pre-washed U2OS, LCL, or HSC cells and electroporated using Lonza SE, SG or P3 Cell Line 4D-Nucleofector™ X Kit (respectively) with the DN100, CA137, or DZ100 program, according to the manufacture's protocol. At 72 hours cells were lysed, and their genomic DNA content was used in a PCR reaction which amplified the corresponding putative genomic targets. Amplicons were subjected to next-generation sequencing (NGS) and the resulting sequences were then used to calculate the percentage of editing events.

### Results

### Activity of 3′ Trimmed Guides

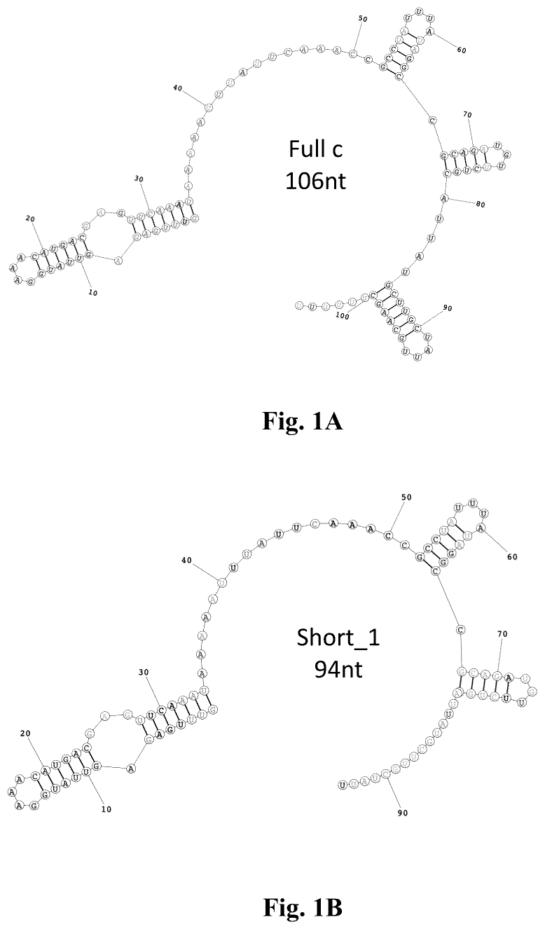

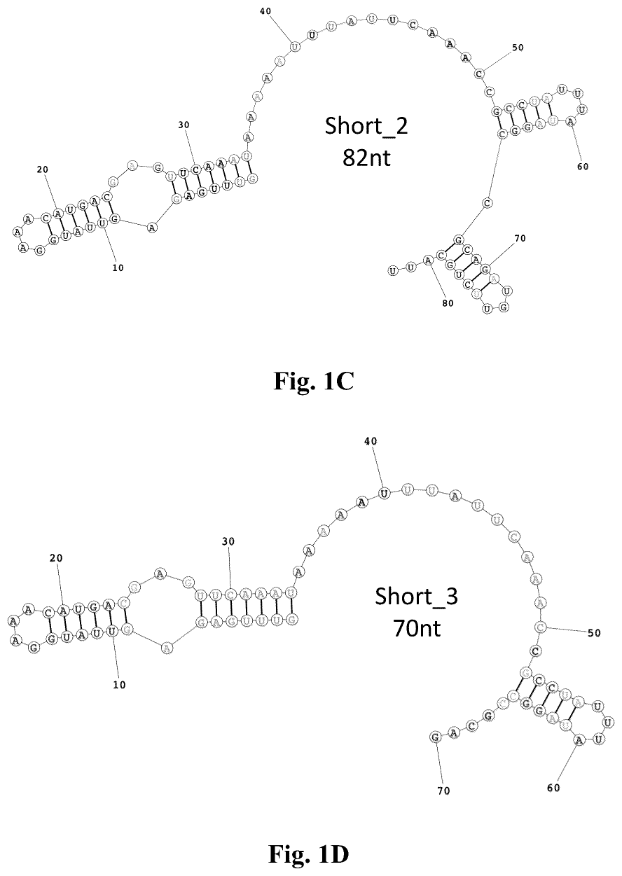

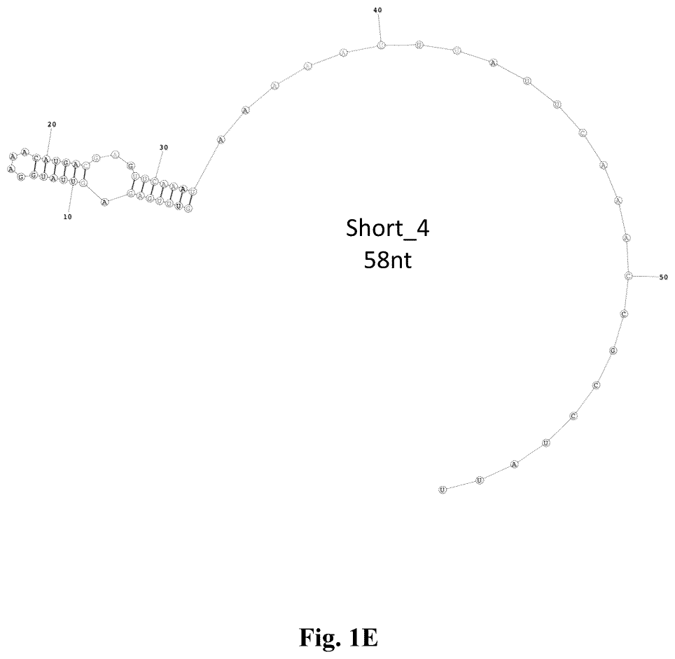

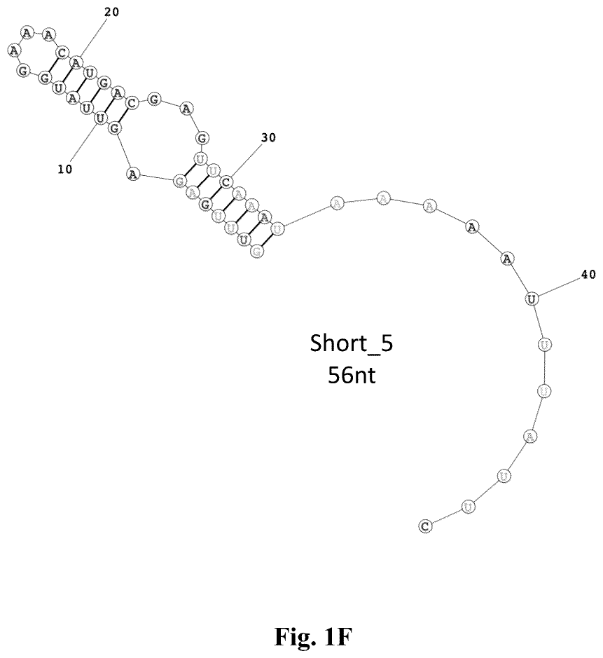

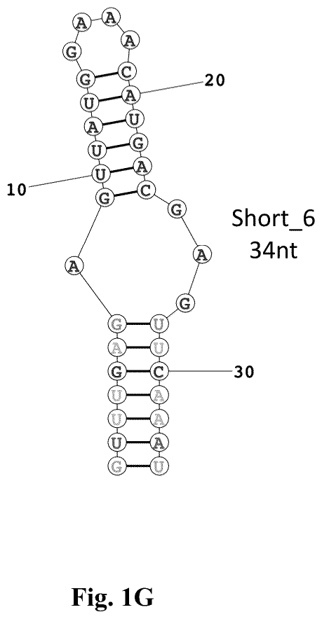

OMNI-50 guide RNAs were optimized by creating shorter scaffold portions. RNA molecules comprising short, non-naturally occurring scaffold portions that minimally sacrifice OMNI-50 nuclease specificity and activity are highly desirable because they reduce complexity of the CRISPR system and permit reliable artificial manufacturing and production of the RNA molecule. First, 3′ trimmed sgRNA scaffolds were tested based on the ‘full c’ duplex version. Six (6) short guides were designed in which the 3′ end was trimmed by 12 nucleotides starting from the full-length guide of OMNI-50 duplex version c (Table 3, FIGS. 1A-1G). The guides were transcribed and vaccinia capped using an IVT kit (GeneJet RNA cleanup and concentration micro kit, ThermoScientific) with a DNA template having a T7 promoter followed by a g35 22-nucleotide spacer (Table 8) and the shortened scaffold designs. Each guide RNA molecule was reacted with purified OMNI-50 nuclease to generate RNPs, tested in vitro for cleavage of the g35 linear template, and tested in vivo for editing of an endogenic g35 site in a U2OS cell line. As can be seen from FIG. 2, RNA molecules having shortened scaffolds up to 46-nucleotides long retained in vitro activity. Furthermore, cell-based editing levels were detected in scaffolds as short as 70 nucleotides.

### Guide Selection for Ranking Guides

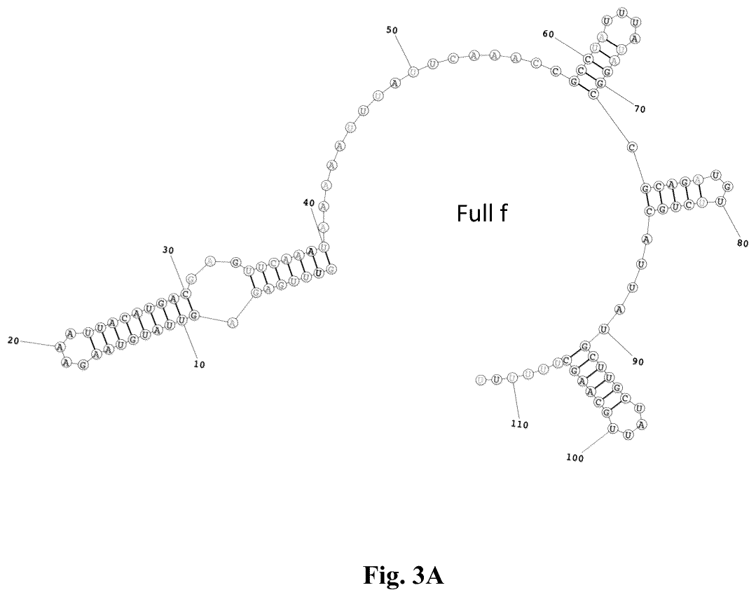

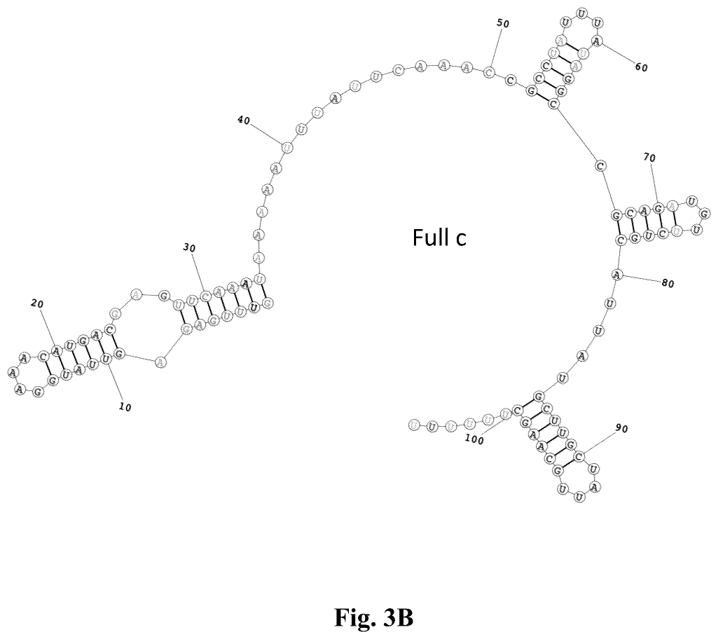

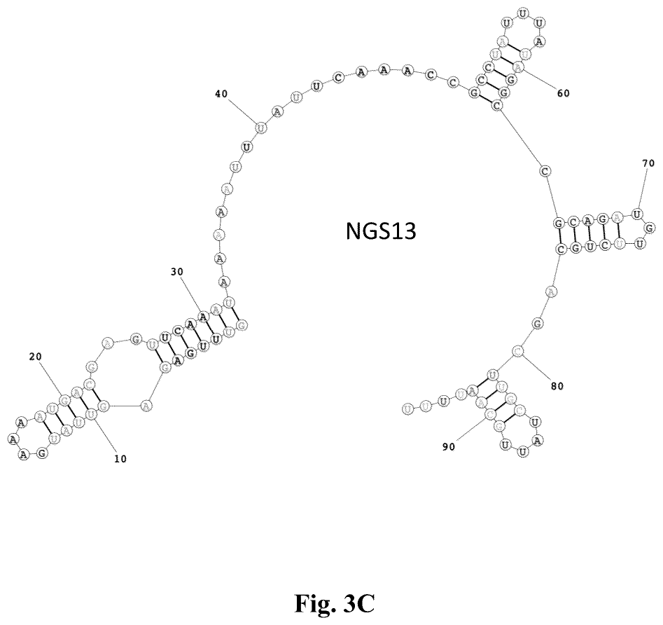

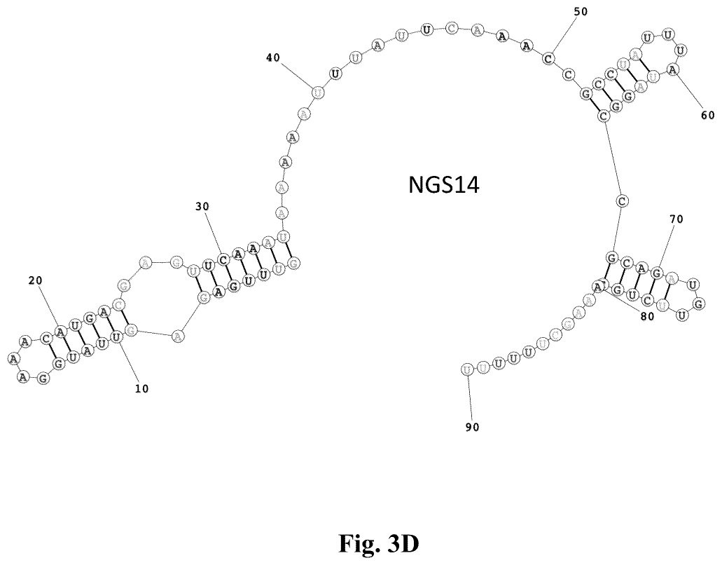

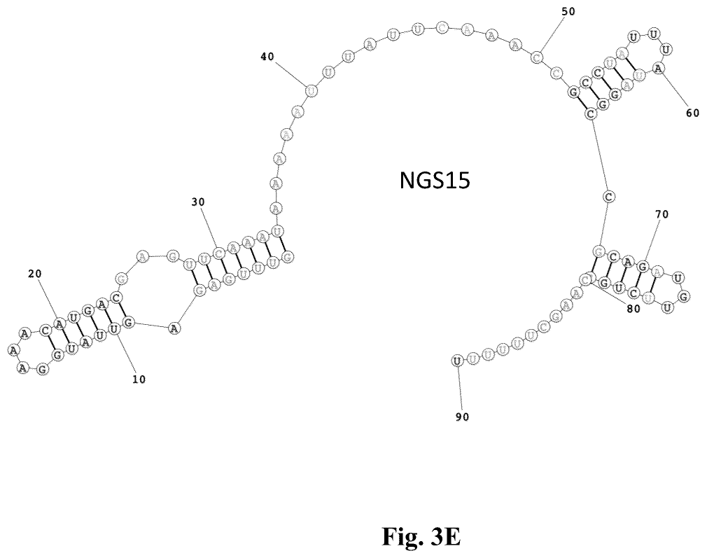

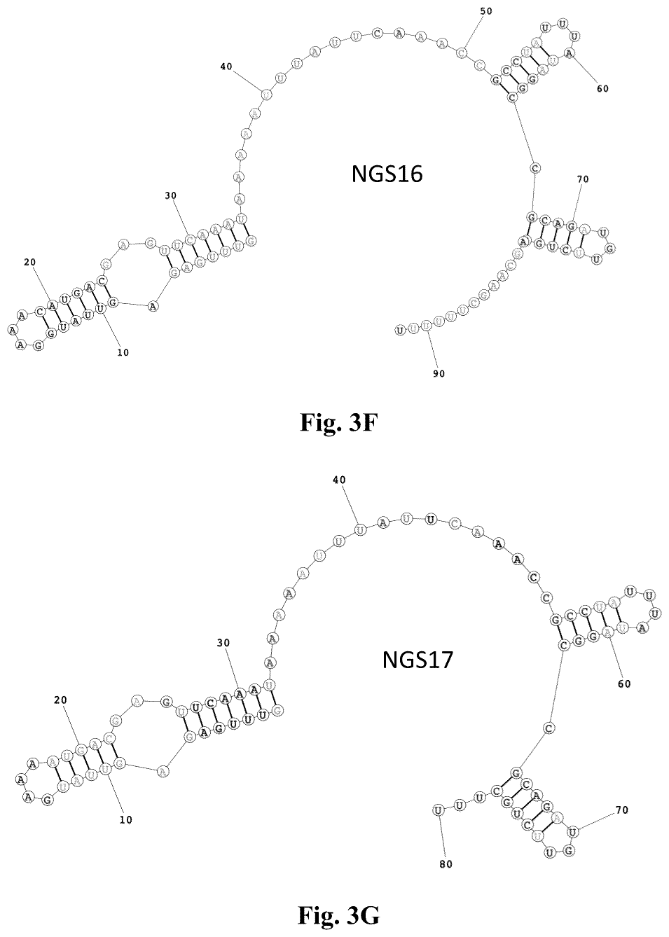

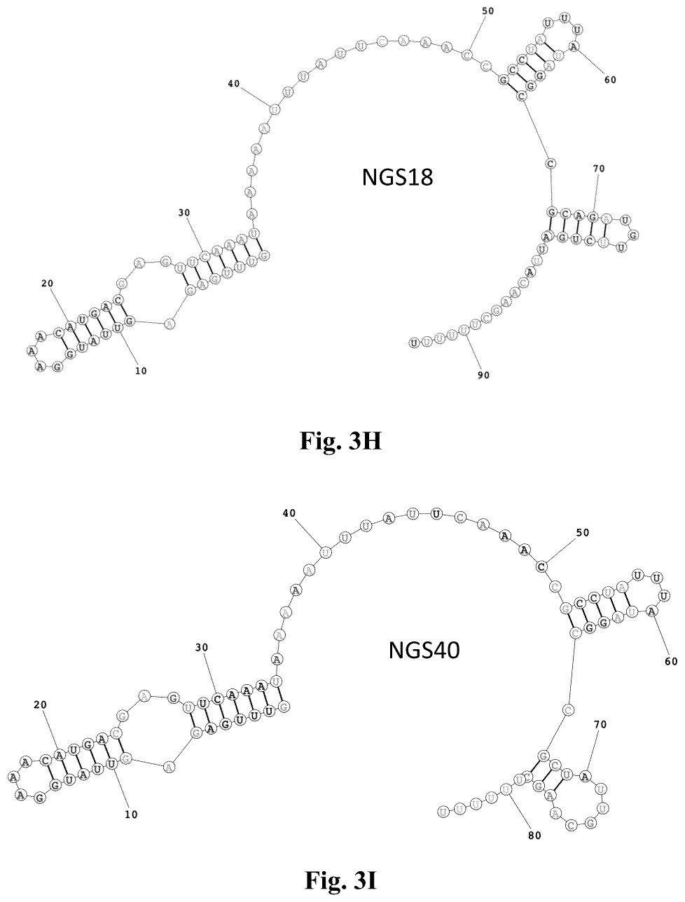

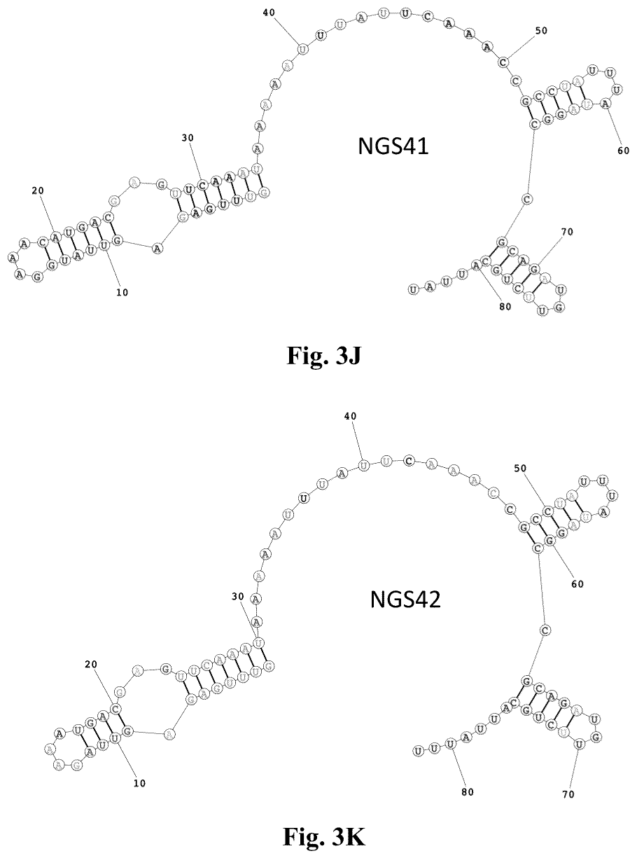

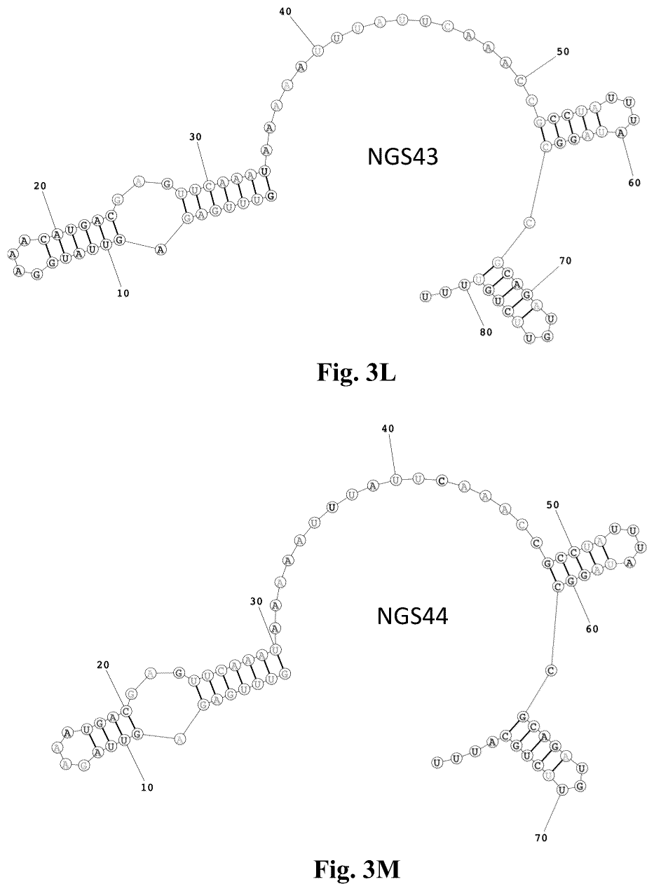

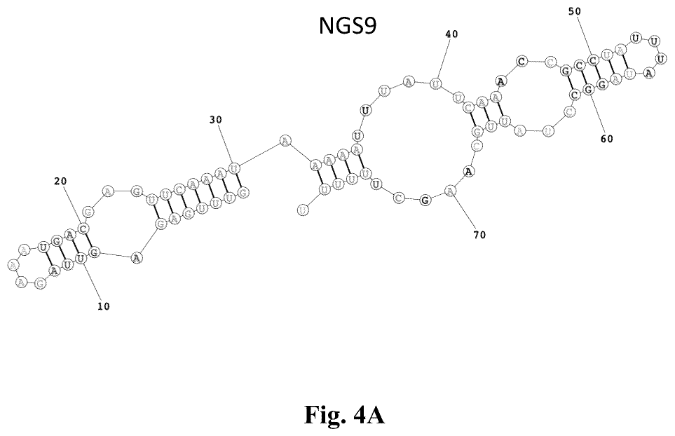

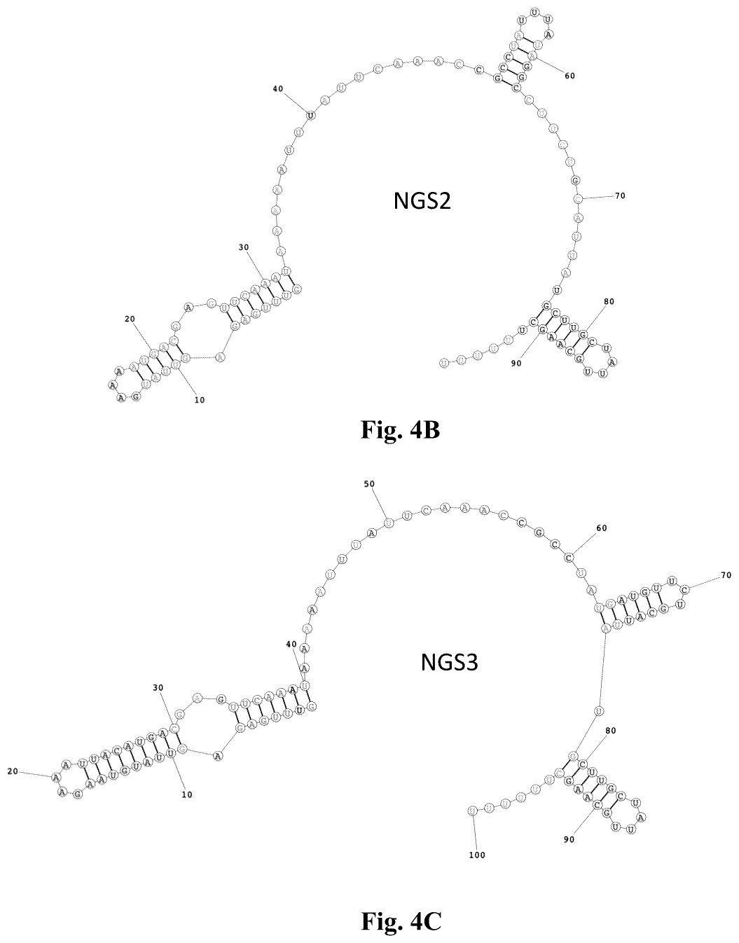

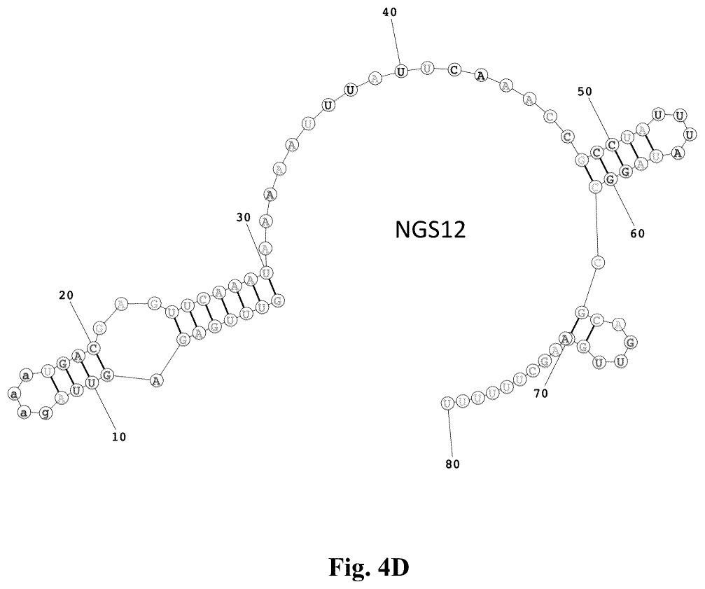

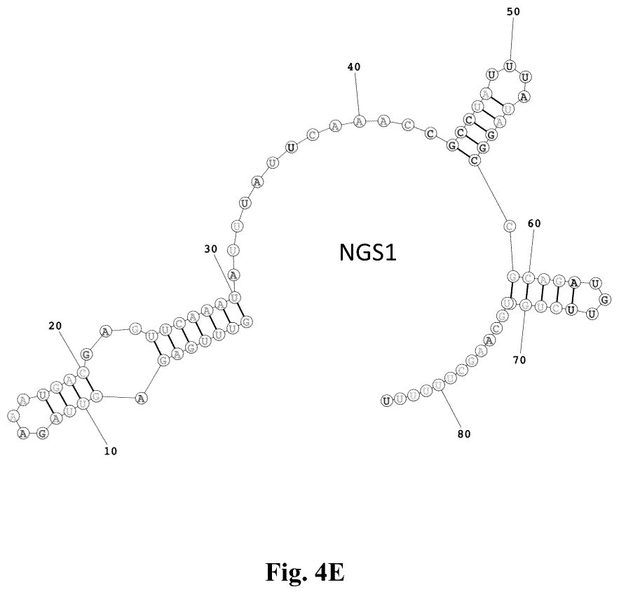

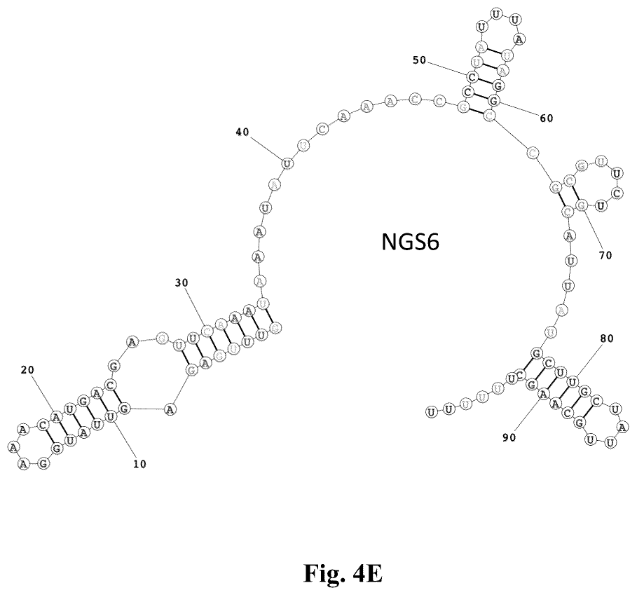

An assay was designed for ranking scaffolds based on their activity. Various scaffolds having deletions along four portions of the scaffold sequence were tested. The scaffold variants were divided into three categories based on their performance in the assay (High, Medium, and Low; Tables 5-7 respectively). Guide variants from the High and the Medium Scoring lists (FIGS. 3 and 4) were also tested for activity using an independent assay as described below.

### Activity of Short Guides Across Genomic Sites and Cell Types

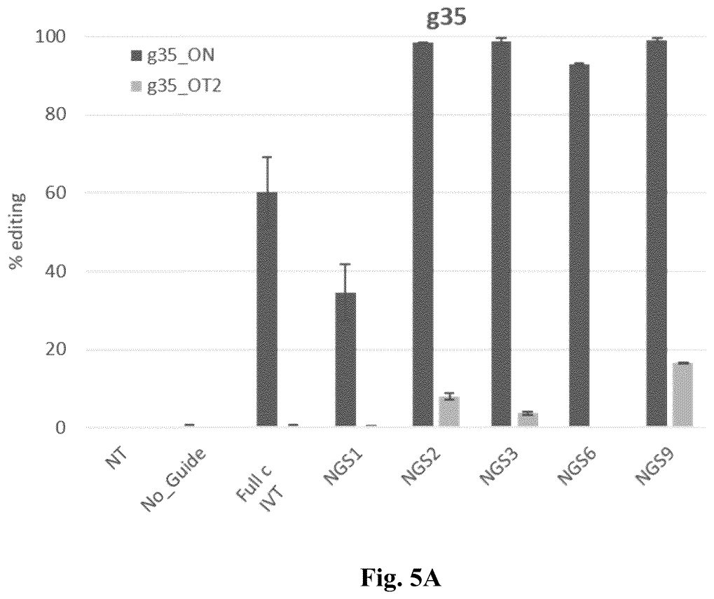

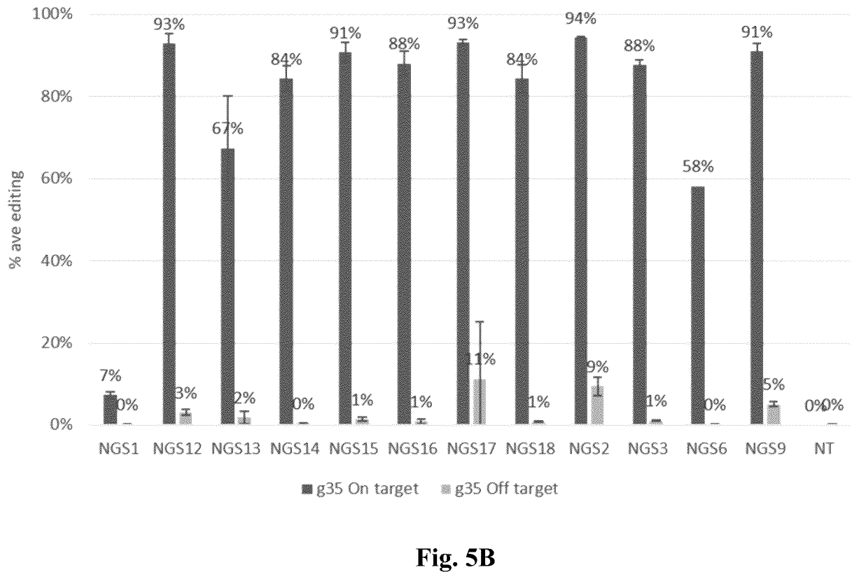

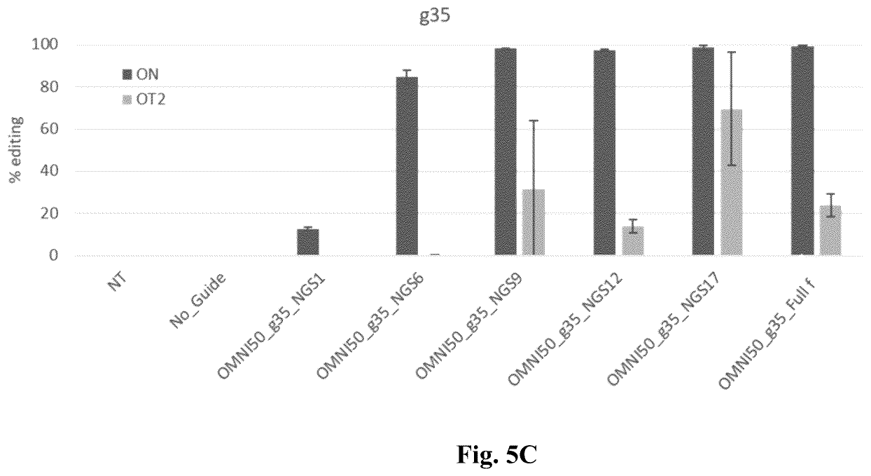

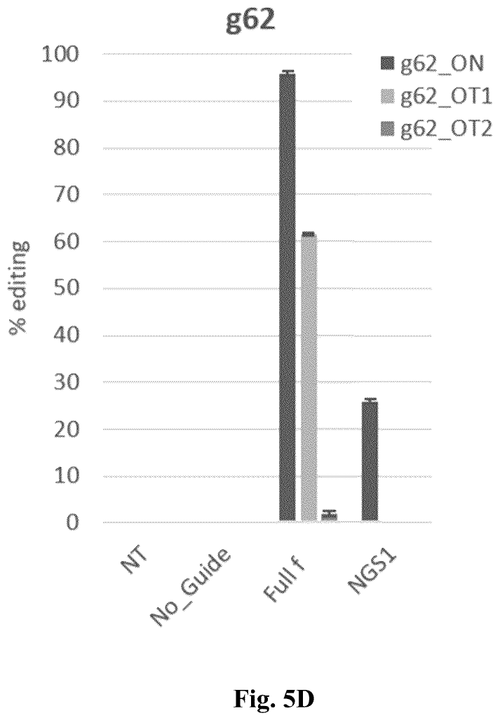

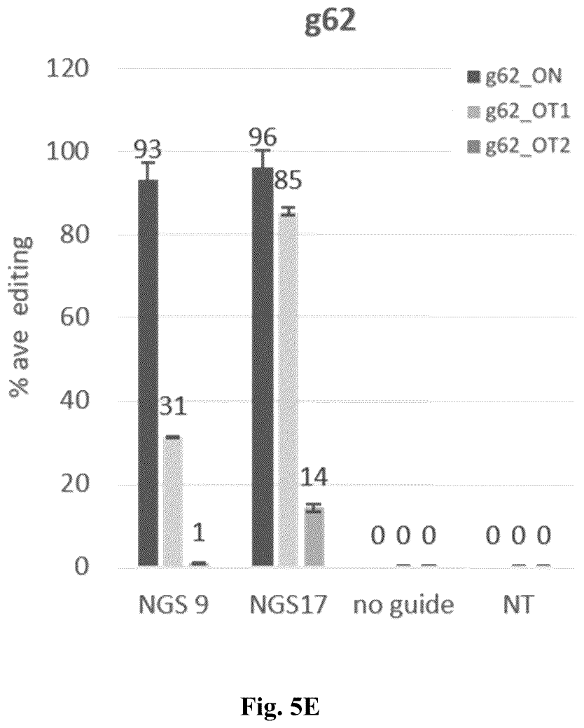

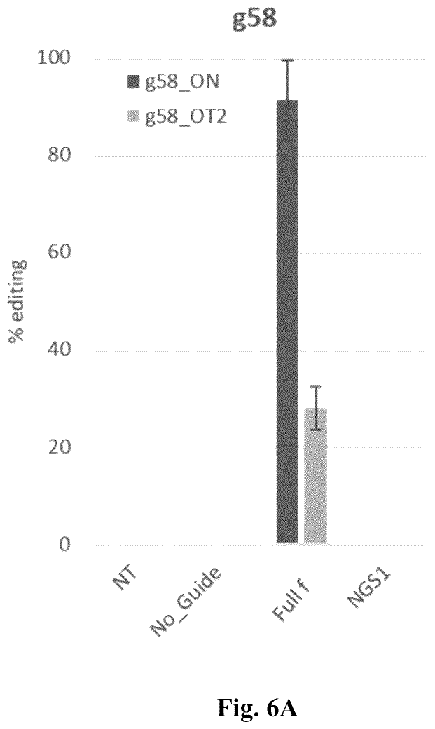

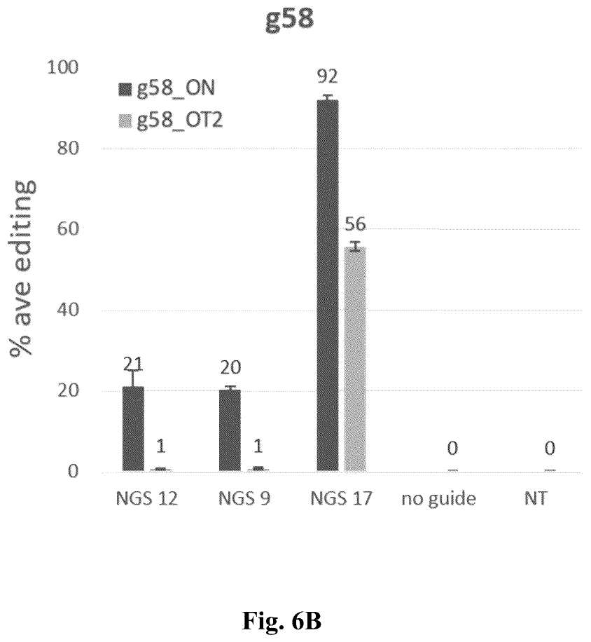

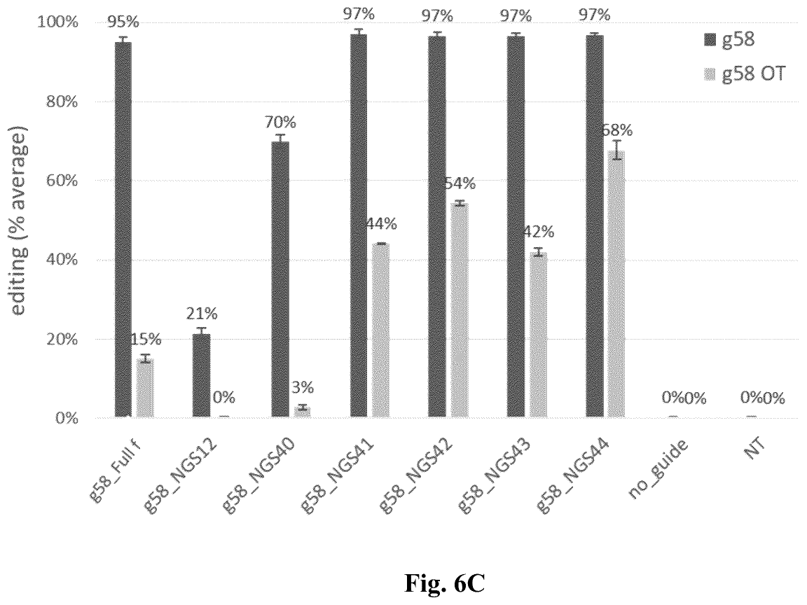

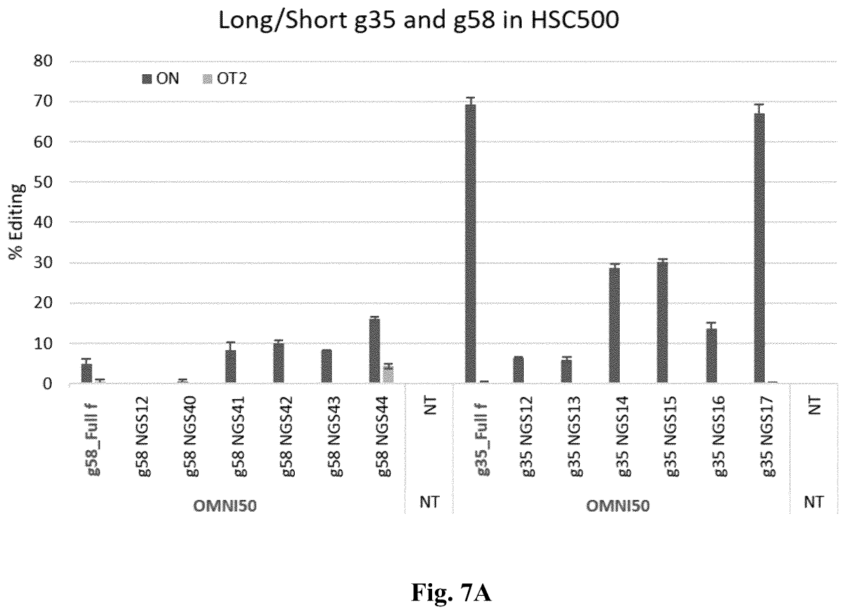

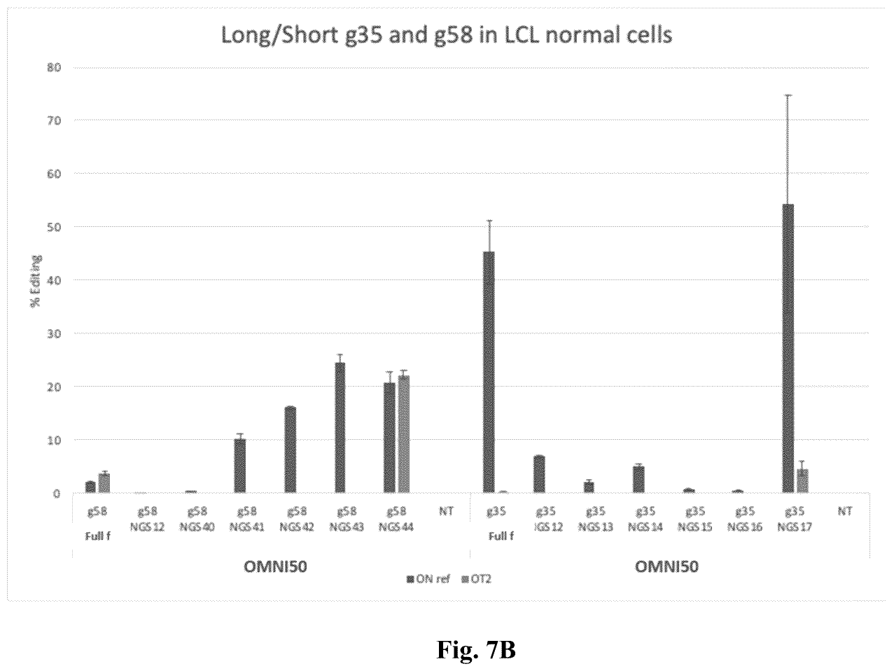

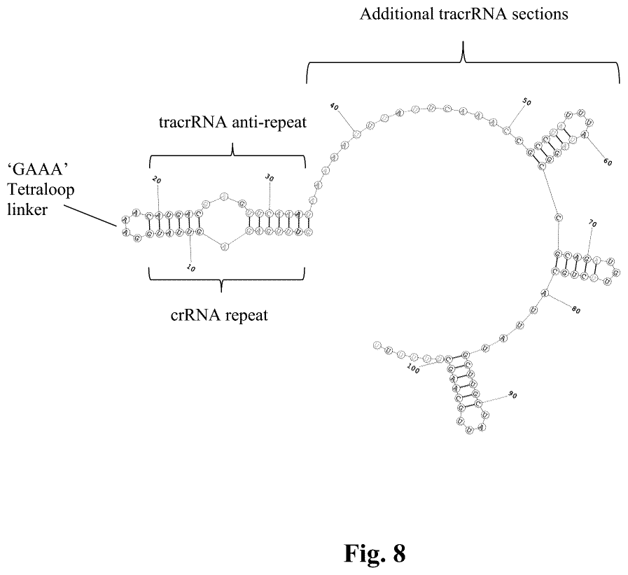

The activity of the short guide RNA molecules on genomic sites was tested with different spacers and on different cell lines (Table 8 -9, FIGS. 5-8). Several optional spacers were chosen, specifically g58, which is a challenging target; and g35 or g62, which are permissive targets. The medium-ranked guides were mostly active on permissive sites (g35 and g62, FIG. 5). However, the high-ranked guides, in particular NGS17 and NGS40-44, proved functional at a challenging site (g58, FIG. 6) and in challenging cell-lines (HSCs and LCLs, FIGS. 7 and 8). An increase in off-target editing using some of the short guide RNA molecules was detected, which imply an increase in the RNP activity with the short guides RNA molecules. As can be seen in Table 5 and Table 9, scaffolds with minimal deletions in the first, second, and third portions retain high activity across genomic sites and cell types.

TABLE 1
OMNI-50 nuclease sequences

| | |
|---|---|
| Nuclease name | OMNI-50 |
| Source organism | Ezakiella peruensis strain M6.X2 |
| Protein sequence | SEQ ID NO: 1 |
| DNA sequence of OMNI-50 ORF | SEQ ID NO: 2 |
| Human optimized DNA sequence of | SEQ ID NO: 3 |
| OMNI-50 ORF | |
| OMNI-50 PAM | NGG |
| sgRNA V1 Scaffold | GUUUGAGAGUUAUGGAAACAUGACG |
| (Experimentally noted as Full c) | AGUUCAAAUAAAAAUUUAUUCAAACC |
| | GCCUAUUUAUAGGCCGCAGAUGUUCU |
| | GCAUUAUGCUUGCUAUUGCAAGCUUU |
| | UUU (SEQ ID NO: 4) |
| sgRNA V2Scaffold | GUUUGAGAGUUAUGUAAGAAAUUAC |
| (Experimentally noted as Full f) | AUGACGAGUUCAAAUAAAAAUUUAU |
| | UCAAACCGCCUAUUUAUAGGCCGCAG |
| | AUGUUCUGCAUUAUGCUUGCUAUUGC |
| | AAGCUUUUUU (SEQ ID NO: 5) |

TABLE 2
Plasmid for OMNI-50 Expression and Purification

| | | Coding ORF | DNA |
|---|---|---|---|
| Plasmid | Elements | Sequence | Sequence |
| pET9a | T7 promoter - SV40 NLS - | SEQ ID | SEQ ID |
| | OMNI ORF (human optimized) - | NO: 6 | NO: 7 |
| | HA - SV40 NLS - 8 His-tag - | | |
| | T7 terminator | | |

TABLE 3
IVT products of 3′ trimming of Full Scaffold C (dashes represent nucleotide deletions
relative to the “Full c” sequence)

| Experimental | | | |
|---|---|---|---|
| Name | crRNA (Repeat) | Linker | tracrRNA (Anti-repeat) |
| Full_c | GUUUGAGAGUUAUG | GAAA | CAUGACGAGUUCAAAU (SEQ ID |
| | (SEQ ID NO: 8) | | NO: 9) |
| Short_1 | GUUUGAGAGUUAUG | GAAA | CAUGACGAGUUCAAAU (SEQ ID |
| | (SEQ ID NO: 8) | | NO: 9) |
| Short_2 | GUUUGAGAGUUAUG | GAAA | CAUGACGAGUUCAAAU (SEQ ID |
| | (SEQ ID NO: 8) | | NO: 9) |
| Short_3 | GUUUGAGAGUUAUG | GAAA | CAUGACGAGUUCAAAU (SEQ ID |
| | (SEQ ID NO: 8) | | NO: 9) |
| Short_4 | GUUUGAGAGUUAUG | GAAA | CAUGACGAGUUCAAAU (SEQ ID |
| | (SEQ ID NO: 8) | | NO: 9) |
| Short_5 | GUUUGAGAGUUAUG | GAAA | CAUGACGAGUUCAAAU (SEQ ID |
| | (SEQ ID NO: 8) | | NO: 9) |
| Short_6 | GUUUGAGAGUUAUG | GAAA | CAUGACGAGUUCAAAU (SEQ ID |
| | (SEQ ID NO: 8) | | NO: 9) |

IVT products of 3′ trimming of Full Scaffold C

| Experimental | First tracrRNA | Second tracrRNA | Third tracrRNA | Fourth tracrRNA |
|---|---|---|---|---|
| Name | Section | Section | Section | Section |
| Full_c | AAAAAUUUAU | GCCUAUUUAUA | CGCAGAUGUUC | AUUAUGCUUGC |
| | UCAAACC (SEQ | GGC (SEQ ID | UGC (SEQ ID | UAUUGCAAGCU |
| | ID NO: 10) | NO: 12) | NO: 13) | UUUUU (SEQ ID |
| | | | | NO: 14) |
| Short_1 | AAAAAUUUAU | GCCUAUUUAUA | CGCAGAUGUUC | AUUAUGCUUGC |
| | UCAAACC (SEQ | GGC (SEQ ID | UGC (SEQ ID | UAUU------------ |
| | ID NO: 10) | NO: 12) | NO: 13) | (SEQ ID NO: 15) |
| Short_2 | AAAAAUUUAU | GCCUAUUUAUA | CGCAGAUGUUC | AUU------------- |
| | UCAAACC (SEQ | GGC (SEQ ID | UGC (SEQ ID | ----------- |
| | ID NO: 10) | NO: 12) | NO: 13) | |
| Short_3 | AAAAAUUUAU | GCCUAUUUAUA | CGCAG--------- | --------------- |
| | UCAAACC (SEQ | GGC (SEQ ID | | ------------ |
| | ID NO: 10) | NO: 12) | | |
| Short_4 | AAAAAUUUAU | GCCUAUU------- | -------------- | --------------- |
| | UCAAACC (SEQ | | | ------------ |
| | ID NO: 10) | | | |
| Short_5 | AAAAAUUUAU | -------------- | -------------- | --------------- |
| | UC----- (SEQ ID | | | ------------ |
| | NO: 11) | | | |
| Short_6 | ----------------- | -------------- | -------------- | --------------- |
| | | | | ------------ |

IVT products of 3′ trimming of Full Scaffold C

| Experimental | | |
|---|---|---|
| Name | Full sequence | Length |
| Full_c | GUUUGAGAGUUAUGGAAACAUGACGAGUUCAAAUAAAAAUUU | 106 |
| | AUUCAAACCGCCUAUUUAUAGGCCGCAGAUGUUCUGCAUUAUG | |
| | CUUGCUAUUGCAAGCUUUUUU (SEQ ID NO: 4) | |
| Short_1 | GUUUGAGAGUUAUGGAAACAUGACGAGUUCAAAUAAAAAUUU | 94 |
| | AUUCAAACCGCCUAUUUAUAGGCCGCAGAUGUUCUGCAUUAUG | |
| | CUUGCUAUU (SEQ ID NO: 16) | |
| Short_2 | GUUUGAGAGUUAUGGAAACAUGACGAGUUCAAAUAAAAAUUU | 82 |
| | AUUCAAACCGCCUAUUUAUAGGCCGCAGAUGUUCUGCAUU | |
| | (SEQ ID NO: 17) | |
| Short_3 | GUUUGAGAGUUAUGGAAACAUGACGAGUUCAAAUAAAAAUUU | 70 |
| | AUUCAAACCGCCUAUUUAUAGGCCGCAG (SEQ ID NO: 18) | |
| Short_4 | GUUUGAGAGUUAUGGAAACAUGACGAGUUCAAAUAAAAAUUU | 58 |
| | AUUCAAACCGCCUAUU (SEQ ID NO: 19) | |
| Short_5 | GUUUGAGAGUUAUGGAAACAUGACGAGUUCAAAUAAAAAUUU | 46 |
| | AUUC (SEQ ID NO: 20) | |
| Short_6 | GUUUGAGAGUUAUGGAAACAUGACGAGUUCAAAU (SEQ ID NO: | 34 |
| | 21) | |

TABLE 4
All designed full scaffold guides with different lengths after the crRNA:tracrRNA duplex
bulge (dashes where present represent nucleotide deletions relative to the “Full pre-processed”
sequence)

| Experimental | | | |
|---|---|---|---|
| Name | crRNA (Repeat) | Linker | tracrRNA (Anti-repeat) |
| Full_pre- | GUUUGAGAGUUAUGUA | GAAA | UUACAUGACGAGUUCAAAU |
| processed | AUUUCAUAUAGGACUA | | (SEQ ID NO: 26) |
| | AAACGAAAGCUAUAAU | | |
| | AUGUAU (SEQ ID NO: 22) | | |
| Full_f | GUUUGAGAGUUAUGUA | GAAA | UUACAUGACGAGUUCAAAU |
| | A------------------------- | | (SEQ ID NO: 26) |
| | ------------ (SEQ ID NO: 23) | | |
| Full_c | GUUUGAGAGUUAUG----- | GAAA | ---CAUGACGAGUUCAAAU (SEQ |
| | --------------------------- | | ID NO: 9) |
| | -------- (SEQ ID NO: 8) | | |
| Full_b | GUUUGAGAGUUAU------- | GAAA | ----AUGACGAGUUCAAAU ((SEQ |
| | -------------------------- | | ID NO: 27) |
| | -------- (SEQ ID NO: 24) | | |
| Full_a | GUUUGAGAGUUA---------- | GAAA | -----UGACGAGUUCAAAU (SEQ ID |
| | ---------------------------- | | NO: 28) |
| | ---- (SEQ ID NO: 25) | | |

All designed full scaffold guides with different lengths after the
crRNA:tracrRNA duplex bulge

| Experimental | First tracrRNA | Second tracrRNA | Third tracrRNA | Fourth tracrRNA |
|---|---|---|---|---|
| Name | Section | Section | Section | Section |
| Full_pre- | AAAAAUUUAU | GCCUAUUUAUA | CGCAGAUGUUC | AUUAUGCUUGC |
| processed | UCAAACC (SEQ | GGC (SEQ ID | UGC (SEQ ID | UAUUGCAAGCU |
| | ID NO: 10) | NO: 12) | NO: 13) | UUUUU (SEQ ID |
| | | | | NO: 14) |
| Full_f | AAAAAUUUAU | GCCUAUUUAUA | CGCAGAUGUUC | AUUAUGCUUGC |
| | UCAAACC (SEQ | GGC (SEQ ID | UGC (SEQ ID | UAUUGCAAGCU |
| | ID NO: 10) | NO: 12) | NO: 13) | UUUUU (SEQ ID |
| | | | | NO: 14) |
| Full_c | AAAAAUUUAU | GCCUAUUUAUA | CGCAGAUGUUC | AUUAUGCUUGC |
| | UCAAACC (SEQ | GGC (SEQ ID | UGC (SEQ ID | UAUUGCAAGCU |
| | ID NO: 10) | NO: 12) | NO: 13) | UUUUU (SEQ ID |
| | | | | NO: 14) |
| Full_b | AAAAAUUUAU | GCCUAUUUAUA | CGCAGAUGUUC | AUUAUGCUUGC |
| | UCAAACC (SEQ | GGC (SEQ ID | UGC (SEQ ID | UAUUGCAAGCU |
| | ID NO: 10) | NO: 12) | NO: 13) | UUUUU (SEQ ID |
| | | | | NO: 14) |
| Full_a | AAAAAUUUAU | GCCUAUUUAUA | CGCAGAUGUUC | AUUAUGCUUGC |
| | UCAAACC (SEQ | GGC (SEQ ID | UGC (SEQ ID | UAUUGCAAGCU |
| | ID NO: 10) | NO: 12) | NO: 13) | UUUUU (SEQ ID |
| | | | | NO: 14) |

All designed full scaffold guides with different lengths after the
crRNA:TracrRNA duplex blulge

| Experimental | |
|---|---|
| Name | Full sequence |
| Full pre- | GUUUGAGAGUUAUGUAAUUUCAUAUAGGACUAAAACgaaaGCUAU |
| processed | AAUAUGUAUGAAAUUACAUGACGAGUUCAAAUAAAAAUUUAUUC |
| | AAACCGCCUAUUUAUAGGCCGCAGAUGUUCUGCAUUAUGCUUGC |
| | UAUUGCAAGCUUUUUU (SEQ ID NO: 29) |
| Full_f | GUUUGAGAGUUAUGUAAGAAAUUACAUGACGAGUUCAAAUAAAA |
| | AUUUAUUCAAACCGCCUAUUUAUAGGCCGCAGAUGUUCUGCAUU |
| | AUGCUUGCUAUUGCAAGCUUUUUU (SEQ ID NO: 5) |
| Full_c | GUUUGAGAGUUAUGGAAACAUGACGAGUUCAAAUAAAAAUUUAU |
| | UCAAACCGCCUAUUUAUAGGCCGCAGAUGUUCUGCAUUAUGCUU |
| | GCUAUUGCAAGCUUUUUU (SEQ ID NO: 4) |
| Full_b | GUUUGAGAGUUAUGAAAAUGACGAGUUCAAAUAAAAAUUUAUUC |
| | AAACCGCCUAUUUAUAGGCCGCAGAUGUUCUGCAUUAUGCUUGC |
| | UAUUGCAAGCUUUUUU (SEQ ID NO: 30) |
| Full_a | GUUUGAGAGUUAGAAAUGACGAGUUCAAAUAAAAAUUUAUUCAA |
| | ACCGCCUAUUUAUAGGCCGCAGAUGUUCUGCAUUAUGCUUGCUA |
| | UUGCAAGCUUUUUU (SEQ ID NO: 31) |

TABLE 5
High ranked short scaffolds (dashes where present represent nucleotide deletions
relative to the “Full c” sequence)

| Scaffold | | | |
|---|---|---|---|
| (Experimental | | | |
| Name) | crRNA (Repeat) | Linker | tracrRNA (Anti-repeat) |
| Full_c | GUUUGAGAGUUAUG | GAAA | CAUGACGAGUUCAAAU (SEQ ID |
| | (SEQ ID NO: 8) | | NO: 9) |
| 01 (NGS_13) | GUUUGAGAGUUAU- | GAAA | -AUGACGAGUUCAAAU (SEQ ID |
| | (SEQ ID NO: 24) | | NO: 27) |
| 02 | GUUUGAGAGUUA-- (SEQ | GAAA | --UGACGAGUUCAAAU (SEQ ID |
| | ID NO: 25) | | NO: 28) |
| 03 | GUUUGAGAGUUAUG | GAAA | CAUGACGAGUUCAAAU (SEQ ID |
| | (SEQ ID NO: 8) | | NO: 9) |
| 04 | GUUUGAGAGUUAUG | GAAA | CAUGACGAGUUCAAAU (SEQ ID |
| | (SEQ ID NO: 8) | | NO: 9) |
| 05 | GUUUGAGAGUUAUG | GAAA | CAUGACGAGUUCAAAU (SEQ ID |
| | (SEQ ID NO: 8) | | NO: 9) |
| 06 (NGS_15) | GUUUGAGAGUUAUG | GAAA | CAUGACGAGUUCAAAU (SEQ ID |
| | (SEQ ID NO: 8) | | NO: 9) |
| 07 | GUUUGAGAGUUAUG | GAAA | CAUGACGAGUUCAAAU (SEQ ID |
| | (SEQ ID NO: 8) | | NO: 9) |
| 08 (NGS_40) | GUUUGAGAGUUAUG | GAAA | CAUGACGAGUUCAAAU (SEQ ID |
| | (SEQ ID NO: 8) | | NO: 9) |
| 09 (NGS_42) | GUUUGAGAGUUA-- (SEQ | GAAA | --UGACGAGUUCAAAU (SEQ ID |
| | ID NO: 25) | | NO: 28) |
| 10 (NGS_44) | GUUUGAGAGUUA-- (SEQ | GAAA | --UGACGAGUUCAAAU (SEQ ID |
| | ID NO: 25) | | NO: 28) |
| 11 | GUUUGAGAGUUAU- | GAAA | --UGACGAGUUCAAAU (SEQ ID |
| | (SEQ ID NO: 24) | | NO: 28) |
| 12 | GUUUGAGAGUUAUG | GAAA | CAUGACGAGUUCAAAU (SEQ ID |
| | (SEQ ID NO: 8) | | NO: 9) |
| 13 | GUUUGAGAGUUAUG | GAAA | CAUGACGAGUUCAAAU (SEQ ID |
| | (SEQ ID NO: 8) | | NO: 9) |
| 14 | GUUUGAGAGUUAUG | GAAA | CAUGACGAGUUCAAAU (SEQ ID |
| | (SEQ ID NO: 8) | | NO: 9) |
| 15 (NGS_16) | GUUUGAGAGUUAUG | GAAA | CAUGACGAGUUCAAAU (SEQ ID |
| | (SEQ ID NO: 8) | | NO: 9) |
| 16 | GUUUGAGAGUUAUG | GAAA | CAUGACGAGUUCAAAU (SEQ ID |
| | (SEQ ID NO: 8) | | NO: 9) |
| 17 | GUUUGAGAGUUA-- (SEQ | GAAA | --UGACGAGUUCAAAU (SEQ ID |
| | ID NO: 25) | | NO: 28) |
| 18 | GUUUGAGAGUUAUG | GAAA | CAUGACGAGUUCAAAU (SEQ ID |
| | (SEQ ID NO: 8) | | NO: 9) |
| 19 | GUUUGAGAGUUAUG | GAAA | CAUGACGAGUUCAAAU (SEQ ID |
| | (SEQ ID NO: 8) | | NO: 9) |
| 20 (NGS_18) | GUUUGAGAGUUAUG | GAAA | CAUGACGAGUUCAAAU (SEQ ID |
| | (SEQ ID NO: 8) | | NO: 9) |
| 21 | GUUUGAGAGUUAUG | GAAA | CAUGACGAGUUCAAAU (SEQ ID |
| | (SEQ ID NO: 8) | | NO: 9) |
| 22 | GUUUGAGAGUUAUG | GAAA | CAUGACGAGUUCAAAU (SEQ ID |
| | (SEQ ID NO: 8) | | NO: 9) |
| 23 | GUUUGAGAGUUAUG | GAAA | CAUGACGAGUUCAAAU (SEQ ID |
| | (SEQ ID NO: 8) | | NO: 9) |
| 24 | GUUUGAGAGUUAUG | GAAA | CAUGACGAGUUCAAAU (SEQ ID |
| | (SEQ ID NO: 8) | | NO: 9) |
| 25 | GUUUGAGAGUUAUG | GAAA | CAUGACGAGUUCAAAU (SEQ ID |
| | (SEQ ID NO: 8) | | NO: 9) |
| 26 | GUUUGAGAGUUAUG | GAAA | CAUGACGAGUUCAAAU (SEQ ID |
| | (SEQ ID NO: 8) | | NO: 9) |
| 27 | GUUUGAGAGUUAU- | GAAA | -- UGACGAGUUCAAAU (SEQ ID |
| | (SEQ ID NO: 24) | | NO: 28) |
| 28 | GUUUGAGAGUUAUG | GAAA | CAUGACGAGUUCAAAU (SEQ ID |
| | (SEQ ID NO: 8) | | NO: 9) |
| 29 | GUUUGAGAGUUAUG | GAAA | CAUGACGAGUUCAAAU (SEQ ID |
| | (SEQ ID NO: 8) | | NO: 9) |
| 30 | GUUUGAGAGUUAUG | GAAA | CAUGACGAGUUCAAAU (SEQ ID |
| | (SEQ ID NO: 8) | | NO: 9) |
| 31 (NGS_14) | GUUUGAGAGUUAUG | GAAA | CAUGACGAGUUCAAAU (SEQ ID |
| | (SEQ ID NO: 8) | | NO: 9) |
| 32 | GUUUGAGAGUUAUG | GAAA | CAUGACGAGUUCAAAU (SEQ ID |
| | (SEQ ID NO: 8) | | NO: 9) |
| 33 (NGS_41) | GUUUGAGAGUUAUG | GAAA | CAUGACGAGUUCAAAU (SEQ ID |
| | (SEQ ID NO: 8) | | NO: 9) |
| 34 | GUUUGAGAGUUAUG | GAAA | CAUGACGAGUUCAAAU (SEQ ID |
| | (SEQ ID NO: 8) | | NO: 9) |
| 35 | GUUUGAGAGUUAUG | GAAA | CAUGACGAGUUCAAAU (SEQ ID |
| | (SEQ ID NO: 8) | | NO: 9) |
| 36 | GUUUGAGAGUUAUG | GAAA | CAUGACGAGUUCAAAU (SEQ ID |
| | (SEQ ID NO: 8) | | NO: 9) |
| 37 | GUUUGAGAGUUAUG | GAAA | CAUGACGAGUUCAAAU (SEQ ID |
| | (SEQ ID NO: 8) | | NO: 9) |
| 38 | GUUUGAGAGUUAUG | GAAA | CAUGACGAGUUCAAAU (SEQ ID |
| | (SEQ ID NO: 8) | | NO: 9) |
| 39 | GUUUGAGAGUUAUG | GAAA | CAUGACGAGUUCAAAU (SEQ ID |
| | (SEQ ID NO: 8) | | NO: 9) |
| 40 | GUUUGAGAGUUA-- (SEQ | GAAA | --UGACGAGUUCAAAU (SEQ ID |
| | ID NO: 25) | | NO: 28) |
| 41 | GUUUGAGAGUUAUG | GAAA | CAUGACGAGUUCAAAU (SEQ ID |
| | (SEQ ID NO: 8) | | NO: 9) |
| 42 | GUUUGAGAGUUAUG | GAAA | CAUGACGAGUUCAAAU (SEQ ID |
| | (SEQ ID NO: 8) | | NO: 9) |
| 43 | GUUUGAGAGUUAUG | GAAA | CAUGACGAGUUCAAAU (SEQ ID |
| | (SEQ ID NO: 8) | | NO: 9) |
| 44 | GUUUGAGAGUUAUG | GAAA | CAUGACGAGUUCAAAU (SEQ ID |
| | (SEQ ID NO: 8) | | NO: 9) |
| 45 | GUUUGAGAGUUAUG | GAAA | CAUGACGAGUUCAAAU (SEQ ID |
| | (SEQ ID NO: 8) | | NO: 9) |
| 46 | GUUUGAGAGUUAUG | GAAA | CAUGACGAGUUCAAAU (SEQ ID |
| | (SEQ ID NO: 8) | | NO: 9) |
| 47 | GUUUGAGAGUUAUG | GAAA | CAUGACGAGUUCAAAU (SEQ ID |
| | (SEQ ID NO: 8) | | NO: 9) |
| 48 | GUUUGAGAGUUAUG | GAAA | CAUGACGAGUUCAAAU (SEQ ID |
| | (SEQ ID NO: 8) | | NO: 9) |
| 49 | GUUUGAGAGUUAUG | GAAA | CAUGACGAGUUCAAAU (SEQ ID |
| | (SEQ ID NO: 8) | | NO: 9) |
| 50 (NGS_43) | GUUUGAGAGUUAUG | GAAA | CAUGACGAGUUCAAAU (SEQ ID |
| | (SEQ ID NO: 8) | | NO: 9) |
| 51 | GUUUGAGAGUUAUG | GAAA | CAUGACGAGUUCAAAU (SEQ ID |
| | (SEQ ID NO: 8) | | NO: 9) |
| 52 | GUUUGAGAGUUAUG | GAAA | CAUGACGAGUUCAAAU (SEQ ID |
| | (SEQ ID NO: 8) | | NO: 9) |
| 53 (NGS_17) | GUUUGAGAGUUAU- | GAAA | -AUGACGAGUUCAAAU (SEQ ID |
| | (SEQ ID NO: 24) | | NO: 27) |

High ranked short scaffolds

| Scaffold | | | | |
|---|---|---|---|---|
| (Experimental | First tracrRNA | Second tracrRNA | Third tracrRNA | Fourth tracrRNA |
| Name) | Section | Section | Section | Section |
| Full_c | AAAAAUUUAU | GCCUAUUUAUA | CGCAGAUGUUC | AUUAUGCUUGC |
| | UCAAACC (SEQ | GGC (SEQ ID | UGC (SEQ ID | UAUUGCAAGCU |
| | ID NO: 10) | NO: 12) | NO: 13) | UUUUU (SEQ ID |
| | | | | NO: 14) |
| 01 (NGS_13) | AAAAAUUUAU | GCCUAUUUAUA | CGCAGAUGUUC | A---- |
| | UCAAACC (SEQ | GGC (SEQ ID | UGC (SEQ ID | GCUUGCUAUUG |
| | ID NO: 10) | NO: 12) | NO: 13) | CAA----UUUU |
| | | | | (SEQ ID NO: 35) |
| 02 | AAAAAUUUAU | GCCUAUUUAUA | CGCAGAUGUUC | AU---------------- |
| | UCAAACC (SEQ | GGC (SEQ ID | UGC (SEQ ID | UAUUUUUUU |
| | ID NO: 10) | NO: 12) | NO: 13) | (SEQ ID NO: 36) |
| 03 | AAAAAUUUAU | GCCUAUUUAUA | CGCAGAUGUUC | AU---- |
| | UCAAACC (SEQ | GGC (SEQ ID | UGC (SEQ ID | UAUGCUUUUGC |
| | ID NO: 10) | NO: 12) | NO: 13) | AA----UUUU |
| | | | | (SEQ ID NO: 37) |
| 04 | AAAAAUUUAU | GCCUAUUUAUA | CGCAGAUGUUC | AU---------- |
| | UCAAACC (SEQ | GGC (SEQ ID | UGC (SEQ ID | UUUGCAAGCUU |
| | ID NO: 10) | NO: 12) | NO: 13) | UUUU (SEQ ID |
| | | | | NO: 38) |
| 05 | AAAAAUUUAU | GCCUAUUUAUA | CGCAGAUGUUC | CU---------- |
| | UCAAACC (SEQ | GGC (SEQ ID | UGC (SEQ ID | AUUGCAAGCUU |
| | ID NO: 10) | NO: 12) | NO: 13) | UUUU (SEQ ID |
| | | | | NO: 39) |
| 06 (NGS_15) | AAAAAUUUAU | GCCUAUUUAUA | CGCAGAUGUUC | ---------------- |
| | UCAAACC (SEQ | GGC (SEQ ID | UGC (SEQ ID | CAAGCUUUUUU |
| | ID NO: 10) | NO: 12) | NO: 13) | (SEQ ID NO: 40) |
| 07 | AAAAAUUUAU | GCCUAUUUAUA | CGCAGAUGUUC | AUUA---- |
| | UCAAACC (SEQ | GGC (SEQ ID | UGC (SEQ ID | UGCUAUUGCA-- |
| | ID NO: 10) | NO: 12) | NO: 13) | --UUUUU (SEQ |
| | | | | ID NO: 41) |
| 08 (NGS_40) | AAAAAUUUAU | GCCUAUUUAUA | CGC----------- | ----------- |
| | UCAAACC (SEQ | GGC (SEQ ID | | UAUUGCAAGCU |
| | ID NO: 10) | NO: 12) | | UUUUU (SEQ ID |
| | | | | NO: 42) |
| 09 (NGS_42) | AAAAAUUUAU | GCCUAUUUAUA | CGCAGAUGUUC | -------------------- |
| | UCAAACC (SEQ | GGC (SEQ ID | UGC (SEQ ID | AUUAUUU |
| | ID NO: 10) | NO: 12) | NO: 13) | |
| 10 (NGS_44) | AAAAAUUUAU | GCCUAUUUAUA | CGCAGAUGUUC | AUU-U------------- |
| | UCAAACC (SEQ | GGC (SEQ ID | UGC (SEQ ID | --------- |
| | ID NO: 10) | NO: 12) | NO: 13) | |
| 11 | AAAAAUUUAU | GCCUAUUUAUA | CGCAGAUGUUC | AA---------------- |
| | UCAAACC (SEQ | GGC (SEQ ID | UGC (SEQ ID | AGCUUUUUU |
| | ID NO: 10) | NO: 12) | NO: 13) | (SEQ ID NO: 43) |
| 12 | AAAAAUUUAU | GCCUAUUUAUA | CGCAGAUGUUC | AU---- |
| | UCAAACC (SEQ | GGC (SEQ ID | UGC (SEQ ID | UUUGCUAUU---- |
| | ID NO: 10) | NO: 12) | NO: 13) | GCUUUUUU |
| | | | | (SEQ ID NO: 44) |
| 13 | AAAAAUUUAU | GCCUAUUUAUA | CGCAGAUGUUC | AG---- |
| | UCAAACC (SEQ | GGC (SEQ ID | UGC (SEQ ID | CUUGCUAUUGC |
| | ID NO: 10) | NO: 12) | NO: 13) | AA----UUUU |
| | | | | (SEQ ID NO: 35) |
| 14 | AAAAAUUUAU | GCCUAUUUAUA | CGCAGAUGUUC | AU------------ |
| | UCAAACC (SEQ | GGC (SEQ ID | UGU (SEQ ID | UGCAAGCUUUU |
| | ID NO: 10) | NO: 12) | NO: 33) | UU (SEQ ID NO: |
| | | | | 45) |
| 15 (NGS_16) | AAAAAUUUAU | GCCUAUUUAUA | CGCAGAUGUUC | AG-------------- |
| | UCAAACC (SEQ | GGC (SEQ ID | UGC (SEQ ID | CAAGCUUUUUU |
| | ID NO: 10) | NO: 12) | NO: 13) | (SEQ ID NO: 46) |
| 16 | AAAAAUUUAU | GCCUAUUUAUA | CGCAGAUGUUC | AU-------- |
| | UCAAACC (SEQ | GGC (SEQ ID | UGC (SEQ ID | UAAUUGCAAGC |
| | ID NO: 10) | NO: 12) | NO: 13) | UUUUUU (SEQ |
| | | | | ID NO: 47) |
| 17 | AAAAAUUUAU | GCCUAUUUAUA | CGCAGAUGUUC | AU-------- |
| | UCAAACC (SEQ | GGC (SEQ ID | UGC (SEQ ID | UAUGCGCAAGC |
| | ID NO: 10) | NO: 12) | NO: 13) | UUUUUU (SEQ |
| | | | | ID NO: 48) |
| 18 | AAAAAUUUAU | GCCUAUUUAUA | CGCAGAUGUUC | AU----------------- |
| | UCAAACC (SEQ | GGC (SEQ ID | UGC (SEQ ID | AGCUUUUUU |
| | ID NO: 10) | NO: 12) | NO: 13) | (SEQ ID NO: 49) |
| 19 | AAAAAUUUAU | GCCUAUUUAUA | CGCAGAUGUUC | AU------ |
| | UCAAACC (SEQ | GGC (SEQ ID | UGC (SEQ ID | UACUAUUGCAA |
| | ID NO: 10) | NO: 12) | NO: 13) | GCUUUUUU |
| | | | | (SEQ ID NO: 50) |
| 20 (NGS_18) | AAAAAUUUAU | GCCUAUUUAUA | CGCAGAUGUUC | AU------------ |
| | UCAAACC (SEQ | GGC (SEQ ID | UGC (SEQ ID | UACAAGCUUUU |
| | ID NO: 10) | NO: 12) | NO: 13) | UU (SEQ ID NO: |
| | | | | 51) |
| 21 | AAAAAUUUAU | GCCUAUUUAUA | CGCAGAUGUUC | AU------ |
| | UCAAACC (SEQ | GGC (SEQ ID | UGC (SEQ ID | UGCUAUUGCAA |
| | ID NO: 10) | NO: 12) | NO: 13) | GCUUUUUU |
| | | | | (SEQ ID NO: 52) |
| 22 | AAAAAUUUAU | GCCUAUUUAUA | CGCAGAUGUUC | AU---------------- |
| | UCAAACC (SEQ | GGC (SEQ ID | UGC (SEQ ID | UAUUUUUUU |
| | ID NO: 10) | NO: 12) | NO: 13) | (SEQ ID NO: 36) |
| 23 | AAAAAUUUAU | GCCUAUUUAUA | CGCAGAUGUUC | -------- |
| | UCAAACC (SEQ | GGC (SEQ ID | UGC (SEQ ID | UGCUUGCUAUA |
| | ID NO: 10) | NO: 12) | NO: 13) | GCUUUUUU |
| | | | | (SEQ ID NO: 53) |
| 24 | AAAAAUUUAU | GCCUAUUUAUA | CGCAGAUGUUC | ------------- |
| | UCAAACC (SEQ | GGC (SEQ ID | UG- (SEQ ID NO: | UUGCAAGCUUU |
| | ID NO: 10) | NO: 12) | 34) | UUU (SEQ ID |
| | | | | NO: 54) |
| 25 | AAAAAUUUAU | GCCUAUUUAUA | CGCAGAUGUUC | AU-------- |
| | UCAAACC (SEQ | GGC (SEQ ID | UGC (SEQ ID | CUAUUGCAAGC |
| | ID NO: 10) | NO: 12) | NO: 13) | UUUUUU (SEQ |
| | | | | ID NO: 55) |
| 26 | AAAAAUUUAU | GCC---- | CGCAGAUGUUC | AU---- |
| | UCAAACC (SEQ | UAUAGGC (SEQ | UGC (SEQ ID | CUUGCUAUUGC |
| | ID NO: 10) | ID NO: 32) | NO: 13) | AAGCUUUUUU |
| | | | | (SEQ ID NO: 56) |
| 27 | AAAAAUUUAU | GCCUAUUUAUA | CGCAGAUGUUC | AU-------------- |
| | UCAAACC (SEQ | GGC (SEQ ID | UGC (SEQ ID | CAAGCUUUUUU |
| | ID NO: 10) | NO: 12) | NO: 13) | (SEQ ID NO: 57) |
| 28 | AAAAAUUUAU | GCCUAUUUAUA | CGCAGAUGUUC | AU------------ |
| | UCAAACC (SEQ | GGC (SEQ ID | UGC (SEQ ID | UAUGCUCUUUU |
| | ID NO: 10) | NO: 12) | NO: 13) | UU (SEQ ID NO: |
| | | | | 58) |
| 29 | AAAAAUUUAU | GCCUAUUUAUA | CGCAGAUGUUC | AU-------------- |
| | UCAAACC (SEQ | GGC (SEQ ID | UGC (SEQ ID | CAAGCUUUUUU |
| | ID NO: 10) | NO: 12) | NO: 13) | (SEQ ID NO: 57) |
| 30 | AAAAAUUUAU | GCCUAUUUAUA | CGCAGAUGUUC | AU |
| | UCAAACC (SEQ | GGC (SEQ ID | UGC (SEQ ID | AUUGCAAGCUU |
| | ID NO: 10) | NO: 12) | NO: 13) | UUUU (SEQ ID |
| | | | | NO: 59) |
| 31 (NGS_14) | AAAAAUUUAU | GCCUAUUUAUA | CGCAGAUGUUC | A---------------- |
| | UCAAACC (SEQ | GGC (SEQ ID | UGC (SEQ ID | AAGCUUUUUU |
| | ID NO: 10) | NO: 12) | NO: 13) | (SEQ ID NO: 43) |
| 32 | AAAAAUUUAU | GCCUAUUUAUA | CGCAGAUGUUC | AU-------------- |
| | UCAAACC (SEQ | GGC (SEQ ID | UGC (SEQ ID | UAUGCUUUUU |
| | ID NO: 10) | NO: 12) | NO: 13) | U (SEQ ID NO: |
| | | | | 60) |
| 33 (NGS_41) | AAAAAUUUAU | GCCUAUUUAUA | CGCAGAUGUUC | AU------------------- |
| | UCAAACC (SEQ | GGC (SEQ ID | UGC (SEQ ID | ---UAU |
| | ID NO: 10) | NO: 12) | NO: 13) | |
| 34 | AAAAAUUUAU | GCCUAUUUAUA | CGCAGAUGUUC | AU---- |
| | UCAAACC (SEQ | GGC (SEQ ID | UGC (SEQ ID | UAUGCUUGCUA |
| | ID NO: 10) | NO: 12) | NO: 13) | UAGCUUUU-- |
| | | | | (SEQ ID NO: 61) |
| 35 | AAAAAUUUAU | GCCUAUUUAUA | CGCAGAUGUUC | UG---- |
| | UCAAACC (SEQ | GGC (SEQ ID | UGC (SEQ ID | CUUGCUAUUGC |
| | ID NO: 10) | NO: 12) | NO: 13) | AA----UUUU |
| | | | | (SEQ ID NO: 62) |
| 36 | AAAAAUUUAU | GCCUAUUUAUA | CGCAGAUGUUC | AU------------ |
| | UCAAACC (SEQ | GGC (SEQ ID | UGC (SEQ ID | UAUAAGCUUU |
| | ID NO: 10) | NO: 12) | NO: 13) | UUU (SEQ ID |
| | | | | NO: 63) |
| 37 | AAAAAUUUAU | GCCUAUUUAUA | CGCAGAUGUUC | AU---------------- |
| | UCAAACC (SEQ | GGC (SEQ ID | UGC (SEQ ID | UACUUUUUU |
| | ID NO: 10) | NO: 12) | NO: 13) | (SEQ ID NO: 64) |
| 38 | AAAAAUUUAU | GCCUAUUUAUA | CGCAGAUGUUC | AU------------------ |
| | UCAAACC (SEQ | GGC (SEQ ID | UGC (SEQ ID | UUUUUUU |
| | ID NO: 10) | NO: 12) | NO: 13) | |
| 39 | AAAAAUUUAU | GCCUAUUUAUA | CGCAGAUGUUC | AU-------------- |
| | UCAAACC (SEQ | GGC (SEQ ID | UGC (SEQ ID | UAAGCUUUUU |
| | ID NO: 10) | NO: 12) | NO: 13) | U (SEQ ID NO: |
| | | | | 65) |
| 40 | AAAAAUUUAU | GCCUAUUUAUA | CGCAGAUGUUC | AU------------ |
| | UCAAACC (SEQ | GGC (SEQ ID | UGC (SEQ ID | UGCAAGCUUUU |
| | ID NO: 10) | NO: 12) | NO: 13) | UU (SEQ ID NO: |
| | | | | 45) |
| 41 | AAAAAUUUAU | GCCUAUUUAUA | CGCAGAUGUUC | AU------ |
| | UCAAACC (SEQ | GGC (SEQ ID | UGC (SEQ ID | UAUUAUUGCA |
| | ID NO: 10) | NO: 12) | NO: 13) | AGCUUUUUU |
| | | | | (SEQ ID NO: 66) |
| 42 | AAAAAUUUAU | GCCUAUUUAUA | CGCAGAUGUUC | AU-------- |
| | UCAAACC (SEQ | GGC (SEQ ID | UGC (SEQ ID | UUAUUGCAAGC |
| | ID NO: 10) | NO: 12) | NO: 13) | UUUUUU (SEQ |
| | | | | ID NO: 67) |
| 43 | AAAAAUUUAU | GCCUAUUUAUA | CGCAGAUGUUC | -------- |
| | UCAAACC (SEQ | GGC (SEQ ID | UGC (SEQ ID | UGCUUGCUCAA |
| | ID NO: 10) | NO: 12) | NO: 13) | GCUUUUUU |
| | | | | (SEQ ID NO: 68) |
| 44 | AAAAAUUUAU | GCCUAUUUAUA | CGCAGAUGUUC | AU-------- |
| | UCAAACC (SEQ | GGC (SEQ ID | UGC (SEQ ID | UUUUUGCAAGC |
| | ID NO: 10) | NO: 12) | NO: 13) | UUUUUU (SEQ |
| | | | | ID NO: 69) |
| 45 | AAAAAUUUAU | GCCUAUUUAUA | CGCAGAUGUUC | -------- |
| | UCAAACC (SEQ | GGC (SEQ ID | UGC (SEQ ID | UGCUUUUGCAA |
| | ID NO: 10) | NO: 12) | NO: 13) | GCUUUUUU |
| | | | | (SEQ ID NO: 70) |
| 46 | AAAAAUUUAU | GCCUAUUUAUA | CGCAGAUGUUC | AU-------- |
| | UCAAACC (SEQ | GGC (SEQ ID | UGC (SEQ ID | UUUGUGCAAGC |
| | ID NO: 10) | NO: 12) | NO: 13) | UUUUUU (SEQ |
| | | | | ID NO: 71) |
| 47 | AAAAAUUUAU | GCCUAUUUAUA | CGCAGG-------- | ---------- |
| | UCAAACC (SEQ | GGC (SEQ ID | | CUAUUGCAAGC |
| | ID NO: 10) | NO: 12) | | UUUUUU (SEQ |
| | | | | ID NO: 39) |
| 48 | AAAAAUUUAU | GCCUAUUUAUA | CGCAGAUGUUC | ------------------ |
| | UCAAACC (SEQ | GGC (SEQ ID | UGC (SEQ ID | AGCUUUUUU |
| | ID NO: 10) | NO: 12) | NO: 13) | |
| 49 | AAAAAUUUAU | GCCUAUUUAUA | CGCAGAUGUUC | AU-------- |
| | UCAAACC (SEQ | GGC (SEQ ID | UGC (SEQ ID | UAUGCUUAAGC |
| | ID NO: 10) | NO: 12) | NO: 13) | UUUUUU (SEQ |
| | | | | ID NO: 72) |
| 50 (NGS_43) | AAAAAUUUAU | GCCUAUUUAUA | CGCAGAUGUUC | -------------------- |
| | UCAAACC (SEQ | GGC (SEQ ID | UG- (SEQ ID NO: | --- |
| | ID NO: 10) | NO: 12) | 34) | UUUU |
| 51 | AAAAAUUUAU | GCCUAUUUAUA | CGCAGAUGUUC | -------------------- |
| | UCAAACC (SEQ | GGC (SEQ ID | UGC (SEQ ID | -- |
| | ID NO: 10) | NO: 12) | NO: 13) | UUUUU |
| 52 | AAAAAUUUAU | GCCUAUUUAUA | CGCAGAUGUUC | -------- |
| | UCAAACC (SEQ | GGC (SEQ ID | UGC (SEQ ID | UGCUAUUGCAA |
| | ID NO: 10) | NO: 12) | NO: 13) | GCUUUUUU |
| | | | | (SEQ ID NO: 73) |
| 53 (NGS_17) | AAAAAUUUAU | GCCUAUUUAUA | CGCAGAUGUUC | ------------------- |
| | UCAAACC (SEQ | GGC (SEQ ID | UGC (SEQ ID | -----UUU |
| | ID NO: 10) | NO: 12) | NO: 13) | |

High ranked short scaffolds

| Scaffold | | |
|---|---|---|
| (Experimental | | |
| Name) | Full sequence | Length |
| Full_c | GUUUGAGAGUUAUGGAAACAUGACGAGUUCAAAUAAAAA | 106 |
| | UUUAUUCAAACCGCCUAUUUAUAGGCCGCAGAUGUUCUGC | |
| | AUUAUGCUUGCUAUUGCAAGCUUUUUU (SEQ ID NO: 4) | |
| 01 (NGS_13) | GUUUGAGAGUUAUGAAAAUGACGAGUUCAAAUAAAAAUU | 96 |
| | UAUUCAAACCGCCUAUUUAUAGGCCGCAGAUGUUCUGCAG | |
| | CUUGCUAUUGCAAUUUU (SEQ ID NO: 74) | |
| 02 | GUUUGAGAGUUAGAAAUGACGAGUUCAAAUAAAAAUUUA | 86 |
| | UUCAAACCGCCUAUUUAUAGGCCGCAGAUGUUCUGCAUUA | |
| | UUUUUUU (SEQ ID NO: 75) | |
| 03 | GUUUGAGAGUUAUGGAAACAUGACGAGUUCAAAUAAAAA | 98 |
| | UUUAUUCAAACCGCCUAUUUAUAGGCCGCAGAUGUUCUGC | |
| | AUUAUGCUUUUGCAAUUUU (SEQ ID NO: 76) | |
| 04 | GUUUGAGAGUUAUGGAAACAUGACGAGUUCAAAUAAAAA | 96 |
| | UUUAUUCAAACCGCCUAUUUAUAGGCCGCAGAUGUUCUGC | |
| | AUUUUGCAAGCUUUUUU (SEQ ID NO: 77) | |
| 05 | GUUUGAGAGUUAUGGAAACAUGACGAGUUCAAAUAAAAA | 96 |
| | UUUAUUCAAACCGCCUAUUUAUAGGCCGCAGAUGUUCUGC | |
| | CUAUUGCAAGCUUUUUU (SEQ ID NO: 78) | |
| 06 (NGS_15) | GUUUGAGAGUUAUGGAAACAUGACGAGUUCAAAUAAAAA | 90 |
| | UUUAUUCAAACCGCCUAUUUAUAGGCCGCAGAUGUUCUGC | |
| | CAAGCUUUUUU (SEQ ID NO: 79) | |
| 07 | GUUUGAGAGUUAUGGAAACAUGACGAGUUCAAAUAAAAA | 98 |
| | UUUAUUCAAACCGCCUAUUUAUAGGCCGCAGAUGUUCUGC | |
| | AUUAUGCUAUUGCAUUUUU (SEQ ID NO: 80) | |
| 08 (NGS_40) | GUUUGAGAGUUAUGGAAACAUGACGAGUUCAAAUAAAAA | 84 |
| | UUUAUUCAAACCGCCUAUUUAUAGGCCGCUAUUGCAAGCU | |
| | UUUUU (SEQ ID NO: 81) | |
| 09 (NGS_42) | GUUUGAGAGUUAGAAAUGACGAGUUCAAAUAAAAAUUUA | 82 |
| | UUCAAACCGCCUAUUUAUAGGCCGCAGAUGUUCUGCAUUA | |
| | UUU (SEQ ID NO: 82) | |
| 10 (NGS_44) | GUUUGAGAGUUAGAAAUGACGAGUUCAAAUAAAAAUUUA | 79 |
| | UUCAAACCGCCUAUUUAUAGGCCGCAGAUGUUCUGCAUUU | |
| | (SEQ ID NO: 83) | |
| 11 | GUUUGAGAGUUAUGAAAUGACGAGUUCAAAUAAAAAUUU | 87 |
| | AUUCAAACCGCCUAUUUAUAGGCCGCAGAUGUUCUGCAAA | |
| | GCUUUUUU (SEQ ID NO: 84) | |
| 12 | GUUUGAGAGUUAUGGAAACAUGACGAGUUCAAAUAAAAA | 98 |
| | UUUAUUCAAACCGCCUAUUUAUAGGCCGCAGAUGUUCUGC | |
| | AUUUUGCUAUUGCUUUUUU (SEQ ID NO: 85) | |
| 13 | GUUUGAGAGUUAUGGAAACAUGACGAGUUCAAAUAAAAA | 98 |
| | UUUAUUCAAACCGCCUAUUUAUAGGCCGCAGAUGUUCUGC | |
| | AGCUUGCUAUUGCAAUUUU (SEQ ID NO: 86) | |
| 14 | GUUUGAGAGUUAUGGAAACAUGACGAGUUCAAAUAAAAA | 94 |
| | UUUAUUCAAACCGCCUAUUUAUAGGCCGCAGAUGUUCUGU | |
| | AUUGCAAGCUUUUUU (SEQ ID NO: 87) | |
| 15 (NGS_16) | GUUUGAGAGUUAUGGAAACAUGACGAGUUCAAAUAAAAA | 92 |
| | UUUAUUCAAACCGCCUAUUUAUAGGCCGCAGAUGUUCUGC | |
| | AGCAAGCUUUUUU (SEQ ID NO: 88) | |
| 16 | GUUUGAGAGUUAUGGAAACAUGACGAGUUCAAAUAAAAA | 98 |
| | UUUAUUCAAACCGCCUAUUUAUAGGCCGCAGAUGUUCUGC | |
| | AUUAAUUGCAAGCUUUUUU (SEQ ID NO: 89) | |
| 17 | GUUUGAGAGUUAGAAAUGACGAGUUCAAAUAAAAAUUUA | 94 |
| | UUCAAACCGCCUAUUUAUAGGCCGCAGAUGUUCUGCAUUA | |
| | UGCGCAAGCUUUUUU (SEQ ID NO: 90) | |
| 18 | GUUUGAGAGUUAUGGAAACAUGACGAGUUCAAAUAAAAA | 90 |
| | UUUAUUCAAACCGCCUAUUUAUAGGCCGCAGAUGUUCUGC | |
| | AUAGCUUUUUU (SEQ ID NO: 91) | |
| 19 | GUUUGAGAGUUAUGGAAACAUGACGAGUUCAAAUAAAAA | 100 |
| | UUUAUUCAAACCGCCUAUUUAUAGGCCGCAGAUGUUCUGC | |
| | AUUACUAUUGCAAGCUUUUUU (SEQ ID NO: 92) | |
| 20 (NGS_18) | GUUUGAGAGUUAUGGAAACAUGACGAGUUCAAAUAAAAA | 94 |
| | UUUAUUCAAACCGCCUAUUUAUAGGCCGCAGAUGUUCUGC | |
| | AUUACAAGCUUUUUU (SEQ ID NO: 93) | |
| 21 | GUUUGAGAGUUAUGGAAACAUGACGAGUUCAAAUAAAAA | 100 |
| | UUUAUUCAAACCGCCUAUUUAUAGGCCGCAGAUGUUCUGC | |
| | AUUGCUAUUGCAAGCUUUUUU (SEQ ID NO: 94) | |
| 22 | GUUUGAGAGUUAUGGAAACAUGACGAGUUCAAAUAAAAA | 90 |
| | UUUAUUCAAACCGCCUAUUUAUAGGCCGCAGAUGUUCUGC | |
| | AUUAUUUUUUU (SEQ ID NO: 95) | |
| 23 | GUUUGAGAGUUAUGGAAACAUGACGAGUUCAAAUAAAAA | 98 |
| | UUUAUUCAAACCGCCUAUUUAUAGGCCGCAGAUGUUCUGC | |
| | UGCUUGCUAUAGCUUUUUU (SEQ ID NO: 96) | |
| 24 | GUUUGAGAGUUAUGGAAACAUGACGAGUUCAAAUAAAAA | 92 |
| | UUUAUUCAAACCGCCUAUUUAUAGGCCGCAGAUGUUCUGU | |
| | UGCAAGCUUUUUU (SEQ ID NO: 97) | |
| 25 | GUUUGAGAGUUAUGGAAACAUGACGAGUUCAAAUAAAAA | 98 |
| | UUUAUUCAAACCGCCUAUUUAUAGGCCGCAGAUGUUCUGC | |
| | AUCUAUUGCAAGCUUUUUU (SEQ ID NO: 98) | |
| 26 | GUUUGAGAGUUAUGGAAACAUGACGAGUUCAAAUAAAAA | 98 |
| | UUUAUUCAAACCGCCUAUAGGCCGCAGAUGUUCUGCAUCU | |
| | UGCUAUUGCAAGCUUUUUU (SEQ ID NO: 99) | |
| 27 | GUUUGAGAGUUAUGAAAUGACGAGUUCAAAUAAAAAUUU | 89 |
| | AUUCAAACCGCCUAUUUAUAGGCCGCAGAUGUUCUGCAUC | |
| | AAGCUUUUUU (SEQ ID NO: 100) | |
| 28 | GUUUGAGAGUUAUGGAAACAUGACGAGUUCAAAUAAAAA | 94 |
| | UUUAUUCAAACCGCCUAUUUAUAGGCCGCAGAUGUUCUGC | |
| | AUUAUGCUCUUUUUU (SEQ ID NO: 101) | |
| 29 | GUUUGAGAGUUAUGGAAACAUGACGAGUUCAAAUAAAAA | 92 |
| | UUUAUUCAAACCGCCUAUUUAUAGGCCGCAGAUGUUCUGC | |
| | AUCAAGCUUUUUU (SEQ ID NO: 102) | |
| 30 | GUUUGAGAGUUAUGGAAACAUGACGAGUUCAAAUAAAAA | 96 |
| | UUUAUUCAAACCGCCUAUUUAUAGGCCGCAGAUGUUCUGC | |
| | AUAUUGCAAGCUUUUUU (SEQ ID NO: 103) | |
| 31 (NGS_14) | GUUUGAGAGUUAUGGAAACAUGACGAGUUCAAAUAAAAA | 90 |
| | UUUAUUCAAACCGCCUAUUUAUAGGCCGCAGAUGUUCUGC | |
| | AAAGCUUUUUU (SEQ ID NO: 104) | |
| 32 | GUUUGAGAGUUAUGGAAACAUGACGAGUUCAAAUAAAAA | 92 |
| | UUUAUUCAAACCGCCUAUUUAUAGGCCGCAGAUGUUCUGC | |
| | AUUAUGCUUUUUU (SEQ ID NO: 105) | |
| 33 (NGS_41) | GUUUGAGAGUUAUGGAAACAUGACGAGUUCAAAUAAAAA | 84 |
| | UUUAUUCAAACCGCCUAUUUAUAGGCCGCAGAUGUUCUGC | |
| | AUUAU (SEQ ID NO: 106) | |
| 34 | GUUUGAGAGUUAUGGAAACAUGACGAGUUCAAAUAAAAA | 100 |
| | UUUAUUCAAACCGCCUAUUUAUAGGCCGCAGAUGUUCUGC | |
| | AUUAUGCUUGCUAUAGCUUUU (SEQ ID NO: 107) | |
| 35 | GUUUGAGAGUUAUGGAAACAUGACGAGUUCAAAUAAAAA | 98 |
| | UUUAUUCAAACCGCCUAUUUAUAGGCCGCAGAUGUUCUGC | |
| | UGCUUGCUAUUGCAAUUUU (SEQ ID NO: 108) | |
| 36 | GUUUGAGAGUUAUGGAAACAUGACGAGUUCAAAUAAAAA | 94 |
| | UUUAUUCAAACCGCCUAUUUAUAGGCCGCAGAUGUUCUGC | |
| | AUUAUAAGCUUUUUU (SEQ ID NO: 109) | |
| 37 | GUUUGAGAGUUAUGGAAACAUGACGAGUUCAAAUAAAAA | 90 |
| | UUUAUUCAAACCGCCUAUUUAUAGGCCGCAGAUGUUCUGC | |
| | AUUACUUUUUU (SEQ ID NO: 110) | |
| 38 | GUUUGAGAGUUAUGGAAACAUGACGAGUUCAAAUAAAAA | 88 |
| | UUUAUUCAAACCGCCUAUUUAUAGGCCGCAGAUGUUCUGC | |
| | AUUUUUUUU (SEQ ID NO: 111) | |
| 39 | GUUUGAGAGUUAUGGAAACAUGACGAGUUCAAAUAAAAA | 92 |
| | UUUAUUCAAACCGCCUAUUUAUAGGCCGCAGAUGUUCUGC | |
| | AUUAAGCUUUUUU (SEQ ID NO: 112) | |
| 40 | GUUUGAGAGUUAGAAAUGACGAGUUCAAAUAAAAAUUUA | 90 |
| | UUCAAACCGCCUAUUUAUAGGCCGCAGAUGUUCUGCAUUG | |
| | CAAGCUUUUUU (SEQ ID NO: 113) | |
| 41 | GUUUGAGAGUUAUGGAAACAUGACGAGUUCAAAUAAAAA | 100 |
| | UUUAUUCAAACCGCCUAUUUAUAGGCCGCAGAUGUUCUGC | |
| | AUUAUUAUUGCAAGCUUUUUU (SEQ ID NO: 114) | |
| 42 | GUUUGAGAGUUAUGGAAACAUGACGAGUUCAAAUAAAAA | 98 |
| | UUUAUUCAAACCGCCUAUUUAUAGGCCGCAGAUGUUCUGC | |
| | AUUUAUUGCAAGCUUUUUU (SEQ ID NO: 115) | |
| 43 | GUUUGAGAGUUAUGGAAACAUGACGAGUUCAAAUAAAAA | 98 |
| | UUUAUUCAAACCGCCUAUUUAUAGGCCGCAGAUGUUCUGC | |
| | UGCUUGCUCAAGCUUUUUU (SEQ ID NO: 116) | |
| 44 | GUUUGAGAGUUAUGGAAACAUGACGAGUUCAAAUAAAAA | 98 |
| | UUUAUUCAAACCGCCUAUUUAUAGGCCGCAGAUGUUCUGC | |
| | AUUUUUUGCAAGCUUUUUU (SEQ ID NO: 117) | |
| 45 | GUUUGAGAGUUAUGGAAACAUGACGAGUUCAAAUAAAAA | 98 |
| | UUUAUUCAAACCGCCUAUUUAUAGGCCGCAGAUGUUCUGC | |
| | UGCUUUUGCAAGCUUUUUU (SEQ ID NO: 118) | |
| 46 | GUUUGAGAGUUAUGGAAACAUGACGAGUUCAAAUAAAAA | 98 |
| | UUUAUUCAAACCGCCUAUUUAUAGGCCGCAGAUGUUCUGC | |
| | AUUUUGUGCAAGCUUUUUU (SEQ ID NO: 119) | |
| 47 | GUUUGAGAGUUAUGGAAACAUGACGAGUUCAAAUAAAAA | 88 |
| | UUUAUUCAAACCGCCUAUUUAUAGGCCGCAGGCUAUUGCA | |
| | AGCUUUUUU (SEQ ID NO: 120) | |
| 48 | GUUUGAGAGUUAUGGAAACAUGACGAGUUCAAAUAAAAA | 88 |
| | UUUAUUCAAACCGCCUAUUUAUAGGCCGCAGAUGUUCUGC | |
| | AGCUUUUUU (SEQ ID NO: 121) | |
| 49 | GUUUGAGAGUUAUGGAAACAUGACGAGUUCAAAUAAAAA | 98 |
| | UUUAUUCAAACCGCCUAUUUAUAGGCCGCAGAUGUUCUGC | |
| | AUUAUGCUUAAGCUUUUUU (SEQ ID NO: 122) | |
| 50 (NGS_43) | GUUUGAGAGUUAUGGAAACAUGACGAGUUCAAAUAAAAA | 82 |
| | UUUAUUCAAACCGCCUAUUUAUAGGCCGCAGAUGUUCUGU | |
| | UUU (SEQ ID NO: 123) | |
| 51 | GUUUGAGAGUUAUGGAAACAUGACGAGUUCAAAUAAAAA | 84 |
| | UUUAUUCAAACCGCCUAUUUAUAGGCCGCAGAUGUUCUGC | |
| | UUUUU (SEQ ID NO: 124) | |
| 52 | GUUUGAGAGUUAUGGAAACAUGACGAGUUCAAAUAAAAA | 98 |
| | UUUAUUCAAACCGCCUAUUUAUAGGCCGCAGAUGUUCUGC | |
| | UGCUAUUGCAAGCUUUUUU (SEQ ID NO: 125) | |
| 53 (NGS_17) | GUUUGAGAGUUAUGAAAAUGACGAGUUCAAAUAAAAAUU | 80 |
| | UAUUCAAACCGCCUAUUUAUAGGCCGCAGAUGUUCUGCUU | |
| | U (SEQ ID NO: 126) | |

TABLE 6
Medium ranked short scaffolds (dashes where present represent nucleotide deletions
relative to the “Full f” sequence)

| Scaffold | | | |
|---|---|---|---|
| (Experimental | | | |
| Name) | crRNA (Repeat) | Linker | tracrRNA (Anti-repeat) |
| Full_f | GUUUGAGAGUUAUGUA | GAAA | UUACAUGACGAGUUCAAAU |
| | A (SEQ ID NO: 23) | | (SEQ ID NO: 26) |
| 52 (NGS_9) | GUUUGAGAGUUA----- | GAAA | -----UGACGAGUUCAAAU (SEQ ID |
| | (SEQ ID NO: 25) | | NO: 28) |
| 53 (NGS_2) | GUUUGAGAGUUAU---- | GAAA | ----AUGACGAGUUCAAAU (SEQ |
| | (SEQ ID NO: 24) | | ID NO: 27) |
| 54 (NGS_3) | GUUUGAGAGUUAUGUA | GAAA | UUACAUGACGAGUUCAAAU |
| | A (SEQ ID NO: 23) | | (SEQ ID NO: 26) |
| 55 (NGS_12) | GUUUGAGAGUUA----- | GAAA | -----UGACGAGUUCAAAU (SEQ ID |
| | (SEQ ID NO: 25) | | NO: 28) |
| 56 (NGS_1) | GUUUGAGAGUUA----- | GAAA | -----UGACGAGUUCAAAU (SEQ ID |
| | (SEQ ID NO: 25) | | NO: 28) |
| 57 (NGS_6) | GUUUGAGAGUUAUG--- | GAAA | ---CAUGACGAGUUCAAAU (SEQ |
| | (SEQ ID NO: 8) | | ID NO: 9) |

Medium ranked short scaffolds

| Scaffold | | | | |
|---|---|---|---|---|
| (Experimental | First tracrRNA | Second tracrRNA | Third tracrRNA | Fourth tracrRNA |
| Name) | Section | Section | Section | Section |
| Full_f | AAAAAUUUAU | GCCUAUUUAUA | CGCAGAUGUUC | AUUAUGCUUGC |
| | UCAAACC (SEQ | GGC (SEQ ID | UGC (SEQ ID | UAUUGCAAGCU |
| | ID NO: 10) | NO: 12) | NO: 13) | UUUUU (SEQ ID |
| | | | | NO: 14) |
| 52 (NGS_9) | AAAAAUUUAU | GCCUAUUUAUA | C------------- | ----------- |
| | UCAAACC (SEQ | GGC (SEQ ID | | UAUUGCAAGCU |
| | ID NO: 10) | NO: 12) | | UUUUU (SEQ ID |
| | | | | NO: 42) |
| 53 (NGS_2) | AAAAAUUUAU | GCCUAUUUAUA | C-------UUCUGC | AUUAUGCUUGC |
| | UCAAACC (SEQ | GGC (SEQ ID | | UAUUGCAAGCU |
| | ID NO: 10) | NO: 12) | | UUUUU (SEQ ID |
| | | | | NO: 14) |
| 54 (NGS_3) | AAAAAUUUAU | GCCUAU-------- | GAUGUUCUGC | AUUAUGCUUGC |
| | UCAAACC (SEQ | | (SEQ ID NO: 129) | UAUUGCAAGCU |
| | ID NO: 10) | | | UUUUU (SEQ ID |
| | | | | NO: 14) |
| 55 (NGS_12) | AAAAAUUUAU | GCCUAUUUAUA | CGCAGUUG------ | CAAGCUUUUUU |
| | UCAAACC (SEQ | GGC (SEQ ID | | (SEQ ID NO: 40) |
| | ID NO: 10) | NO: 12) | | |
| 56 (NGS_1) | ---- | GCCUAUUUAUA | CGCAGAUGUUC | -------------- |
| | AUUUAUUCAA | GGC (SEQ ID | UGC (SEQ ID | UGCAAGCUUUU |
| | ACC (SEQ ID | NO: 12) | NO: 13) | UU (SEQ ID NO: |
| | NO: 127) | | | 131) |
| 57 (NGS_6) | AAA---- | GCCUAUUUAUA | CGC---- | AUUAUGCUUGC |
| | UAUUCAAACC | GGC (SEQ ID | GUUCUGC (SEQ | UAUUGCAAGCU |
| | (SEQ ID NO: | NO: 12) | ID NO: 130) | UUUUU (SEQ ID |
| | 128) | | | NO: 14) |

Medium ranked short scaffolds

| Scaffold | |
|---|---|
| (Experimental | |
| Name) | Full sequence |
| Full_f | GUUUGAGAGUUAUGUAAGAAAUUACAUGACGAGUUCAAAUAAAAAUU |
| | UAUUCAAACCGCCUAUUUAUAGGCCGCAGAUGUUCUGCAUUAUGCUUG |
| | CUAUUGCAAGCUUUUUU (SEQ ID NO: 5) |
| 52 (NGS_9) | GUUUGAGAGUUAGAAAUGACGAGUUCAAAUAAAAAUUUAUUCAAACC |
| | GCCUAUUUAUAGGCCUAUUGCAAGCUUUUUU (SEQ ID NO: 132) |
| 53 (NGS_2) | GUUUGAGAGUUAUGAAAAUGACGAGUUCAAAUAAAAAUUUAUUCAAA |
| | CCGCCUAUUUAUAGGCCUUCUGCAUUAUGCUUGCUAUUGCAAGCUUUU |
| | UU (SEQ ID NO: 133) |
| 54 (NGS_3) | GUUUGAGAGUUAUGUAAGAAAUUACAUGACGAGUUCAAAUAAAAAUU |
| | UAUUCAAACCGCCUAUGAUGUUCUGCAUUAUGCUUGCUAUUGCAAGCU |
| | UUUUU (SEQ ID NO: 134) |
| 55 (NGS_12) | GUUUGAGAGUUAgaaaUGACGAGUUCAAAUAAAAAUUUAUUCAAACCGC |
| | CUAUUUAUAGGCCGCAGUUGCAAGCUUUUUU (SEQ ID NO: 135) |
| 56 (NGS_1) | GUUUGAGAGUUAGAAAUGACGAGUUCAAAUAUUUAUUCAAACCGCCU |
| | AUUUAUAGGCCGCAGAUGUUCUGCUGCAAGCUUUUUU (SEQ ID NO: 136) |
| 57 (NGS_6) | GUUUGAGAGUUAUGGAAACAUGACGAGUUCAAAUAAAUAUUCAAACC |
| | GCCUAUUUAUAGGCCGCGUUCUGCAUUAUGCUUGCUAUUGCAAGCUUU |
| | UUU (SEQ ID NO: 137) |

TABLE 7
Low ranked short scaffolds (dashes where present represent nucleotide deletions relative to
the ″Full f″' sequence)

| Scaffold | | | |
|---|---|---|---|
| (Experimental | | | |
| Name) | crRNA (Repeat) | Linker | tracrRNA (Anti-repeat) |
| Full_f | GUUUGAGAGUUAUGUA | GAAA | UUACAUGACGAGUUCAAAU |
| | A (SEQ ID NO: 23) | | (SEQ ID NO: 26) |
| 58 | GUUUGAGAGUUAUG--- | GAAA | ---CAUGACGAGUUCAAAU (SEQ |
| | (SEQ ID NO: 8) | | ID NO: 9) |
| 59 | GUUUGAGAGUUAUG--- | GAAA | ---CAUGACGAGUUCAAAU (SEQ |
| | (SEQ ID NO: 8) | | ID NO: 9) |
| 60 | GUUUGAGAGUUAUG--- | GAAA | ---CAUGACGAGUUCAAAU (SEQ |
| | (SEQ ID NO: 8) | | ID NO: 9) |
| 61 | GUUUGAGAGUUAUG--- | GAAA | ---CAUGACGAGUUCAAAU (SEQ |
| | (SEQ ID NO: 8) | | ID NO: 9) |
| 62 | GUUUGAGAGUUAUG--- | GAAA | ---CAUGACGAGUUCAAAU (SEQ |
| | (SEQ ID NO: 8) | | ID NO: 9) |
| 63 | GUUUGAGAGUUAUG--- | GAAA | ---CAUGACGAGUUCAAAU (SEQ |
| | (SEQ ID NO: 8) | | ID NO: 9) |
| 64 | GUUUGAGAGUUAUG--- | GAAA | ---CAUGACGAGUUCAAAU (SEQ |
| | (SEQ ID NO: 8) | | ID NO: 9) |
| 65 | GUUUGAGAGUUAUG--- | GAAA | ---CAUGACGAGUUCAAAU (SEQ |
| | (SEQ ID NO: 8) | | ID NO: 9) |
| 66 | GUUUGAGAGUUA----- | GAAA | -----UGACGAGUUCAAAU (SEQ ID |
| | (SEQ ID NO: 25) | | NO: 28) |
| 67 | GUUUGAGAGUUAUG--- | GAAA | ---CAUGACGAGUUCAAAU (SEQ |
| | (SEQ ID NO: 8) | | ID NO: 9) |
| 68 | GUUUGAGAGUUAUG--- | GAAA | ---CAUGACGAGUUCAAAU (SEQ |
| | (SEQ ID NO: 8) | | ID NO: 9) |
| 69 | GUUUGAGAGUUAUG--- | GAAA | ---CAUGACGAGUUCAAAU (SEQ |
| | (SEQ ID NO: 8) | | ID NO: 9) |
| 70 | GUUUGAGAGUUAUG--- | GAAA | ---CAUGACGAGUUCAAAU (SEQ |
| | (SEQ ID NO: 8) | | ID NO: 9) |
| 71 | GUUUGAGAGUUAUG--- | GAAA | ---CAUGACGAGUUCAAAU (SEQ |
| | (SEQ ID NO: 8) | | ID NO: 9) |
| 72 | GUUUGAGAGUUAUG--- | GAAA | ---CAUGACGAGUUCAAAU (SEQ |
| | (SEQ ID NO: 8) | | ID NO: 9) |
| 73 | GUUUGAGAGUUAU---- | GAAA | ----AUGACGAGUUCAAAU (SEQ |
| | (SEQ ID NO: 24) | | ID NO: 27) |
| 74 | GUUUGAGAGUUAUG--- | GAAA | ---CAUGACGAGUUCAAAU (SEQ |
| | (SEQ ID NO: 8) | | ID NO: 9) |
| 75 | GUUUGAGAGUUAUG--- | GAAA | ---CAUGACGAGUUCAAAU (SEQ |
| | (SEQ ID NO: 8) | | ID NO: 9) |
| 76 | GUUUGAGAGUUA----- | GAAA | ----UGACGAGUUCAAAUA (SEQ |
| | (SEQ ID NO: 25) | | ID NO: 138) |
| 77 | GUUUGAGAGUUAUG--- | GAAA | ---CAUGACGAGUUCAAAU (SEQ |
| | (SEQ ID NO: 8) | | ID NO: 9) |

Low ranked short scaffolds

| Scaffold | | | | |
|---|---|---|---|---|
| (Experimental | First tracrRNA | Second tracrRNA | Third tracrRNA | Fourth tracrRNA |
| Name) | Section | Section | Section | Section |
| Full_f | AAAAAUUUAU | GCCUAUUUAUA | CGCAGAUGUUC | AUUAUGCUUGC |
| | UCAAACC (SEQ | GGC (SEQ ID | UGC (SEQ ID | UAUUGCAAGCU |
| | ID NO: 10) | NO: 12) | NO: 13) | UUUUU (SEQ ID |
| | | | | NO: 14) |
| 58 | AAA---- | GCCUAUUUAUA | CGCAGAUGUUC | AUUAUGCUUGC |
| | AAUUCAAACC | GGC (SEQ ID | UGC (SEQ ID | UAUUGCAAGCU |
| | (SEQ ID NO: | NO: 12) | NO: 13) | UUUUU (SEQ ID |
| | 139) | | | NO: 14) |
| 59 | AAU---- | GCCUAUUUAUA | CGCAGAUGUUC | AUUAUGCUUGC |
| | UAUUCAAACC | GGC (SEQ ID | UGC (SEQ ID | UAUUGCAAGCU |
| | (SEQ ID NO: | NO: 12) | NO: 13) | UUUUU (SEQ ID |
| | 140) | | | NO: 14) |
| 60 | AUU---- | GCCUAUUUAUA | CGCAGAUGUUC | AUUAUGCUUGC |
| | UAUUCAAACC | GGC (SEQ ID | UGC (SEQ ID | UAUUGCAAGCU |
| | (SEQ ID NO: | NO: 12) | NO: 13) | UUUUU (SEQ ID |
| | 127) | | | NO: 14) |
| 61 | AAC | GCCUAUUUAUA | CGCAGAUGUUC | AUUAUGCUUGC |
| | -------------- | GGC (SEQ ID | UGC (SEQ ID | UAUUGCAAGCU |
| | | NO: 12) | NO: 13) | UUUUU (SEQ ID |
| | | | | NO: 14) |
| 62 | AAA---- | GCCUAUUUAUA | CGCAGAUGUUC | AUUAUGCUUGC |
| | UAUUCAAACC | GGC (SEQ ID | UGC (SEQ ID | UAUUGCAAGCU |
| | (SEQ ID NO: | NO: 12) | NO: 13) | UUUUU (SEQ ID |
| | 128) | | | NO: 14) |
| 63 | AAA | -AAUGGC | CGCAGAUGUUC | AUUAUGCUUGC |
| | -------------- | | UGC (SEQ ID | UAUUGCAAGCU |
| | | | NO: 13) | UUUUU (SEQ ID |
| | | | | NO: 14) |
| 64 | AAA---- | GCCUAUUUAUA | CGCAGAUGUUC | AUUAUGCUUGC |
| | AAUUUAAACC | GGC (SEQ ID | UGC (SEQ ID | UAUUGCAAGCU |
| | (SEQ ID NO: | NO: 12) | NO: 13) | UUUUU (SEQ ID |
| | 141) | | | NO: 14) |
| 65 | AAA---- | GCCUAUUUAUA | CGCAGAUGUUC | AUUAUGCUUGC |
| | AAUUUAUUCC | GGC (SEQ ID | UGC (SEQ ID | UAUUGCAAGCU |
| | (SEQ ID NO: | NO: 12) | NO: 13) | UUUUU (SEQ ID |
| | 142) | | | NO: 14) |
| 66 | A | ---------AAAGGC | CGCAGAUGUUC | AUUAUGCUUGC |
| | ---------------- | | UGC (SEQ ID | UAUUGCAAGCU |
| | | | NO: 13) | UUUUU (SEQ ID |
| | | | | NO: 14) |
| 67 | A | - | CGCAGAUGUUC | AUUAUGCUUGC |
| | ---------------- | CCUAUUUAUAG | UGC (SEQ ID | UAUUGCAAGCU |
| | | GC (SEQ ID NO: | NO: 13) | UUUUU (SEQ ID |
| | | 144) | | NO: 14) |
| 68 | AAA | GCCUAUUUAUA | CGCAGAUGUUC | AUUAUGCUUGC |
| | -------------- | GGC (SEQ ID | UGC (SEQ ID | UAUUGCAAGCU |
| | | NO: 12) | NO: 13) | UUUUU (SEQ ID |
| | | | | NO: 14) |
| 69 | A | -- | CGCAGAUGUUC | AUUAUGCUUGC |
| | ---------------- | CUAUUUAUAG | UGC (SEQ ID | UAUUGCAAGCU |
| | | GC (SEQ ID NO: | NO: 13) | UUUUU (SEQ ID |
| | | 145) | | NO: 14) |
| 70 | AA | -------UAUAGGC | CGCAGAUGUUC | AUUAUGCUUGC |
| | --------------- | | UGC (SEQ ID | UAUUGCAAGCU |
| | | | NO: 13) | UUUUU (SEQ ID |
| | | | | NO: 14) |
| 71 | A | --------AUAGGC | CGCAGAUGUUC | AUUAUGCUUGC |
| | ---------------- | | UGC (SEQ ID | UAUUGCAAGCU |
| | | | NO: 13) | UUUUU (SEQ ID |
| | | | | NO: 14) |
| 72 | AAA---- | GCCUAUUUAUA | CGCAGAUGUUC | AUUAUGCUUGC |
| | AAUUUAUUCA | GGC (SEQ ID | UGC (SEQ ID | UAUUGCAAGCU |
| | (SEQ ID NO: | NO: 12) | NO: 13) | UUUUU (SEQ ID |
| | 143) | | | NO: 14) |
| 73 | A-------------- | GCCUAUUUAUA | CGCAGAUGUUC | AUUAUGCUUGC |
| | CC | GGC (SEQ ID | UGC (SEQ ID | UAUUGCAAGCU |
| | | NO: 12) | NO: 13) | UUUUU (SEQ ID |
| | | | | NO: 14) |
| 74 | ACA----AA------ | GCCUAUUUAUA | CGCAGAUGUUC | AUUAUGCUUGC |
| | CC | GGC (SEQ ID | UGC (SEQ ID | UAUUGCAAGCU |
| | | NO: 12) | NO: 13) | UUUUU (SEQ ID |
| | | | | NO: 14) |
| 75 | AAAAAUUUAU | GCCUAUUUAUA | --- | AUUAUGCUUGC |
| | UCAAACC (SEQ | GG- (SEQ ID NO: | AGAUGUUCUGC | UAUUGCAAGCU |
| | ID NO: 10) | 146) | (SEQ ID NO: 147) | UUUUU (SEQ ID |
| | | | | NO: 14) |
| 76 | ----------------- | --------AUAGGC | CGCAGAUGUUC | AUUAUGCUUGC |
| | | | UGC (SEQ ID | UAUUGCAAGCU |
| | | | NO: 13) | UUUUU (SEQ ID |
| | | | | NO: 14) |
| 77 | AAAAAUUUAU | GCCU---------- | ---- | AUUAUGCUUGC |
| | UCAAACC (SEQ | | GAUGUUCUGC | UAUUGCAAGCU |
| | ID NO: 10) | | (SEQ ID NO: 129) | UUUUU (SEQ ID |
| | | | | NO: 14) |

Low ranked short scaffolds

| Scaffold | |
|---|---|
| (Experimental | |
| Name) | Full sequence |
| Full_f | GUUUGAGAGUUAUGUAAGAAAUUACAUGACGAGUUCAAAUAAAAAUU |
| | UAUUCAAACCGCCUAUUUAUAGGCCGCAGAUGUUCUGCAUUAUGCUUG |
| | CUAUUGCAAGCUUUUUU (SEQ ID NO: 5) |
| 58 | GUUUGAGAGUUAUGGAAACAUGACGAGUUCAAAUAAAAAUUCAAACC |
| | GCCUAUUUAUAGGCCGCAGAUGUUCUGCAUUAUGCUUGCUAUUGCAAG |
| | CUUUUUU (SEQ ID NO: 148) |
| 59 | GUUUGAGAGUUAUGGAAACAUGACGAGUUCAAAUAAUUAUUCAAACC |
| | GCCUAUUUAUAGGCCGCAGAUGUUCUGCAUUAUGCUUGCUAUUGCAAG |
| | CUUUUUU (SEQ ID NO: 149) |
| 60 | GUUUGAGAGUUAUGGAAACAUGACGAGUUCAAAUAUUUAUUCAAACC |
| | GCCUAUUUAUAGGCCGCAGAUGUUCUGCAUUAUGCUUGCUAUUGCAAG |
| | CUUUUUU (SEQ ID NO: 150) |
| 61 | GUUUGAGAGUUAUGGAAACAUGACGAGUUCAAAUAACGCCUAUUUAU |
| | AGGCCGCAGAUGUUCUGCAUUAUGCUUGCUAUUGCAAGCUUUUUU |
| | (SEQ ID NO: 151) |
| 62 | GUUUGAGAGUUAUGGAAACAUGACGAGUUCAAAUAAAUAUUCAAACC |
| | GCCUAUUUAUAGGCCGCAGAUGUUCUGCAUUAUGCUUGCUAUUGCAAG |
| | CUUUUUU (SEQ ID NO: 152) |
| 63 | GUUUGAGAGUUAUGGAAACAUGACGAGUUCAAAUAAAAAUGGCCGCA |
| | GAUGUUCUGCAUUAUGCUUGCUAUUGCAAGCUUUUUU (SEQ ID NO: |
| | 153) |
| 64 | GUUUGAGAGUUAUGGAAACAUGACGAGUUCAAAUAAAAAUUUAAACC |
| | GCCUAUUUAUAGGCCGCAGAUGUUCUGCAUUAUGCUUGCUAUUGCAAG |
| | CUUUUUU (SEQ ID NO: 154) |
| 65 | GUUUGAGAGUUAUGGAAACAUGACGAGUUCAAAUAAAAAUUUAUUCC |
| | GCCUAUUUAUAGGCCGCAGAUGUUCUGCAUUAUGCUUGCUAUUGCAAG |
| | CUUUUUU (SEQ ID NO: 155) |
| 66 | GUUUGAGAGUUAGAAAUGACGAGUUCAAAUAAAAGGCCGCAGAUGUU |
| | CUGCAUUAUGCUUGCUAUUGCAAGCUUUUUU (SEQ ID NO: 156) |
| 67 | GUUUGAGAGUUAUGGAAACAUGACGAGUUCAAAUAACCUAUUUAUAG |
| | GCCGCAGAUGUUCUGCAUUAUGCUUGCUAUUGCAAGCUUUUUU (SEQ |
| | ID NO: 157) |
| 68 | GUUUGAGAGUUAUGGAAACAUGACGAGUUCAAAUAAAACGCCUAUUU |
| | AUAGGCCGCAGAUGUUCUGCAUUAUGCUUGCUAUUGCAAGCUUUUUU |
| | (SEQ ID NO: 158) |
| 69 | GUUUGAGAGUUAUGGAAACAUGACGAGUUCAAAUACUAUUUAUAGGC |
| | CGCAGAUGUUCUGCAUUAUGCUUGCUAUUGCAAGCUUUUUU (SEQ ID |
| | NO: 159) |
| 70 | GUUUGAGAGUUAUGGAAACAUGACGAGUUCAAAUAAUAUAGGCCGCA |
| | GAUGUUCUGCAUUAUGCUUGCUAUUGCAAGCUUUUUU (SEQ ID NO: |
| | 160) |
| 71 | GUUUGAGAGUUAUGGAAACAUGACGAGUUCAAAUAAUAGGCCGCAGA |
| | UGUUCUGCAUUAUGCUUGCUAUUGCAAGCUUUUUU (SEQ ID NO: 161) |
| 72 | GUUUGAGAGUUAUGGAAACAUGACGAGUUCAAAUAAAAAUUUAUUCA |
| | GCCUAUUUAUAGGCCGCAGAUGUUCUGCAUUAUGCUUGCUAUUGCAAG |
| | CUUUUUU (SEQ ID NO: 162) |
| 73 | GUUUGAGAGUUAUGAAAAUGACGAGUUCAAAUACCGCCUAUUUAUAG |
| | GCCGCAGAUGUUCUGCAUUAUGCUUGCUAUUGCAAGCUUUUUU (SEQ |
| | ID NO: 163) |
| 74 | GUUUGAGAGUUAUGGAAACAUGACGAGUUCAAAUACAAACCGCCUAU |
| | UUAUAGGCCGCAGAUGUUCUGCAUUAUGCUUGCUAUUGCAAGCUUUUU |
| | U (SEQ ID NO: 164) |
| 75 | GUUUGAGAGUUAUGGAAACAUGACGAGUUCAAAUAAAAAUUUAUUCA |
| | AACCGCCUAUUUAUAGGAGAUGUUCUGCAUUAUGCUUGCUAUUGCAAG |
| | CUUUUUU (SEQ ID NO: 165) |
| 76 | GUUUGAGAGUUAGAAAUGACGAGUUCAAAUAAUAGGCCGCAGAUGUU |
| | CUGCAUUAUGCUUGCUAUUGCAAGCUUUUUU (SEQ ID NO: 166) |
| 77 | GUUUGAGAGUUAUGGAAACAUGACGAGUUCAAAUAAAAAUUUAUUCA |
| | AACCGCCUGAUGUUCUGCAUUAUGCUUGCUAUUGCAAGCUUUUUU |
| | (SEQ ID NO: 167) |

TABLE 8
Targets for testing activity short-scaffold guide activity

| | DNA coding | | | |
|---|---|---|---|---|
| Guide name | sequence of | | | |
| and target type | Spacer | On target | OT1 | OT2 |
| g35-Permissive | GCAGTCCGGG | GCAGTCCGGG | GCAGACAGGG | ACAGTCCTGGC |
| | CTGGGAGCGG | CTGGGAGCGG | CCAGGAGCGG | TGGGAGCAGGT |
| | GT (SEQ ID NO: | GTGGG (SEQ ID | GAGGG (SEQ ID | GGG (SEQ ID |
| | 168) | NO: 175) | NO: 182) | NO: 187) |
| g58- | CAGCTGCGGG | CAGCTGCGGG | GTGGGGCGGG | CAGCTGAGGGA |
| Challenging | AAAGGGATTC | AAAGGGATTC | AAAGGGAGTC | AAGGAATTCCC |
| | CC (SEQ ID NO: | CCAGG (SEQ ID | CCAGG (SEQ ID | AGG (SEQ ID |
| | 169) | NO: 176) | NO: 183) | NO: 184) |
| g58_alt- | CAGCTGCGGG | CAGCTGCGGG | CAGCTGAGGG | CGCCTGTGGGA |
| Permissive | AATGGGATTCC | AATGGGATTCC | AAAGGAATTC | ATGGGCTTCCC |
| | C (SEQ ID NO: | CAGG (SEQ ID | CCAGG (SEQ ID | CGG (SEQ ID |
| | 170) | NO: 177) | NO: 184) | NO: 188) |
| g62-Permissive | GTGTCAAGCCC | GTGTCAAGCCC | AAGCCAAACC | TGGCCAAGCCT |
| | CAGAGGCCAC | CAGAGGCCAC | CCAAAGGCCA | CAGAGGCCACA |
| | A (SEQ ID NO: | AGGG (SEQ ID | CACGG (SEQ ID | GGG (SEQ ID |
| | 171) | NO: 178) | NO: 185) | NO: 189) |
| g62 Alt- | GTGTCAAGCCC | GTGTCAAGCCC | AAGACAAGAC | TCTTCAAGCCCC |
| Challenging | CAGAGGACAC | CAGAGGACAC | CCAGAGGACA | AGAGGACACTG |
| | A (SEQ ID NO: | AGGG (SEQ ID | CAGGG (SEQ ID | GG (SEQ ID NO: |
| | 172) | NO: 179) | NO: 186) | 190) |
| g39- | CACAGCGGGT | CACAGCGGGT | N/A | N/A |
| Challenging | GTAGACTCCGA | GTAGACTCCGA | | |
| | G (SEQ ID NO: | GGGG (SEQ ID | | |
| | 173) | NO: 180) | | |
| g39alt - | CACAGCaGGTG | CACAGCaGGTG | N/A | N/A |
| Challenging | TAGACTCCGAG | TAGACTCCGAG | | |
| | (SEQ ID NO: 174) | GGG (SEQ ID | | |
| | | NO: 181) | | |

TABLE 9
Targets for testing activity short-scaffold guide activity

| Activity | Scaffold | | Length |
|---|---|---|---|
| Ranking | name | Scaffold sequence | (scaffold + spacer) |
| N/A | Full f | GUUUGAGAGUUAUGUAAGAAAUUACAUGA | 134 |
| | | CGAGUUCAAAUAAAAAUUUAUUCAAACCG | |
| | | CCUAUUUAUAGGCCGCAGAUGUUCUGCAU | |
| | | UAUGCUUGCUAUUGCAAGCUUUUUU (SEQ | |
| | | ID NO: 5) | |
| Medium | NGS_1 | GUUUGAGAGUUAGAAAUGACGAGUUCAAA | 106 |
| Ranked | | UAUUUAUUCAAACCGCCUAUUUAUAGGCC | |
| | | GCAGAUGUUCUGCUGCAAGCUUUUUU (SEQ | |
| | | ID NO: 136) | |
| | NGS_2 | GUUUGAGAGUUAUGAAAAUGACGAGUUCA | 119 |
| | | AAUAAAAAUUUAUUCAAACCGCCUAUUUA | |
| | | UAGGCCUUCUGCAUUAUGCUUGCUAUUGC | |
| | | AAGCUUUUUU (SEQ ID NO: 133) | |
| | NGS_3 | GUUUGAGAGUUAUGUAAGAAAUUACAUGA | 122 |
| | | CGAGUUCAAAUAAAAAUUUAUUCAAACCG | |
| | | CCUAUGAUGUUCUGCAUUAUGCUUGCUAU | |
| | | UGCAAGCUUUUUU (SEQ ID NO: 134) | |
| | NGS_6 | GUUUGAGAGUUAUGGAAACAUGACGAGUU | 120 |
| | | CAAAUAAAUAUUCAAACCGCCUAUUUAUA | |
| | | GGCCGCGUUCUGCAUUAUGCUUGCUAUUG | |
| | | CAAGCUUUUUU (SEQ ID NO: 137) | |
| | NGS_9 | GUUUGAGAGUUAGAAAUGACGAGUUCAAA | 100 |
| | | UAAAAAUUUAUUCAAACCGCCUAUUUAUA | |
| | | GGCCUAUUGCAAGCUUUUUU (SEQ ID NO: | |
| | | 132) | |
| | NGS_12 | GUUUGAGAGUUAgaaaUGACGAGUUCAAAU | 102 |
| | | AAAAAUUUAUUCAAACCGCCUAUUUAUAG | |
| | | GCCGCAGUUGCAAGCUUUUUU (SEQ ID NO: | |
| | | 135) | |
| High | NGS_13 | GUUUGAGAGUUAUGAAAAUGACGAGUUCA | 118 |
| Ranked | | AAUAAAAAUUUAUUCAAACCGCCUAUUUA | |
| | | UAGGCCGCAGAUGUUCUGCAGCUUGCUAU | |
| | | UGCAAUUUU (SEQ ID NO: 74) | |
| | NGS_14 | GUUUGAGAGUUAUGGAAACAUGACGAGUU | 112 |
| | | CAAAUAAAAAUUUAUUCAAACCGCCUAUU | |
| | | UAUAGGCCGCAGAUGUUCUGCAAAGCUUU | |
| | | UUU (SEQ ID NO: 104) | |
| | NGS_15 | GUUUGAGAGUUAUGGAAACAUGACGAGUU | 112 |
| | | CAAAUAAAAAUUUAUUCAAACCGCCUAUU | |
| | | UAUAGGCCGCAGAUGUUCUGCCAAGCUUU | |
| | | UUU (SEQ ID NO: 79) | |
| | NGS_16 | GUUUGAGAGUUAUGGAAACAUGACGAGUU | 114 |
| | | CAAAUAAAAAUUUAUUCAAACCGCCUAUU | |
| | | UAUAGGCCGCAGAUGUUCUGCAGCAAGCU | |
| | | UUUUU (SEQ ID NO: 88) | |
| | NGS_17 | GUUUGAGAGUUAUGAAAAUGACGAGUUCA | 102 |
| | | AAUAAAAAUUUAUUCAAACCGCCUAUUUA | |
| | | UAGGCCGCAGAUGUUCUGCUUU (SEQ ID | |
| | | NO: 126) | |
| | NGS_18 | GUUUGAGAGUUAUGGAAACAUGACGAGUU | 116 |
| | | CAAAUAAAAAUUUAUUCAAACCGCCUAUU | |
| | | UAUAGGCCGCAGAUGUUCUGCAUUACAAG | |
| | | CUUUUUU (SEQ ID NO: 93) | |
| | NGS_40 | GUUUGAGAGUUAUGGAAACAUGACGAGUU | 106 |
| | | CAAAUAAAAAUUUAUUCAAACCGCCUAUU | |
| | | UAUAGGCCGCUAUUGCAAGCUUUUUU (SEQ | |
| | | ID NO: 81) | |
| | NGS_41 | GUUUGAGAGUUAUGGAAACAUGACGAGUU | 106 |
| | | CAAAUAAAAAUUUAUUCAAACCGCCUAUU | |
| | | UAUAGGCCGCAGAUGUUCUGCAUUAU (SEQ | |
| | | ID NO: 106) | |
| | NGS_42 | GUUUGAGAGUUAGAAAUGACGAGUUCAAA | 104 |
| | | UAAAAAUUUAUUCAAACCGCCUAUUUAUA | |
| | | GGCCGCAGAUGUUCUGCAUUAUUU (SEQ ID | |
| | | NO: 82) | |
| | NGS_43 | GUUUGAGAGUUAUGGAAACAUGACGAGUU | 104 |
| | | CAAAUAAAAAUUUAUUCAAACCGCCUAUU | |
| | | UAUAGGCCGCAGAUGUUCUGUUUU (SEQ ID | |
| | | NO: 123) | |
| | NGS_44 | GUUUGAGAGUUAGAAAUGACGAGUUCAAA | 101 |
| | | UAAAAAUUUAUUCAAACCGCCUAUUUAUA | |
| | | GGCCGCAGAUGUUCUGCAUUU (SEQ ID NO: | |
| | | 83) | |

Targets for testing activity short-scaffold guide activity

| | Permissive guide: | | | |
|---|---|---|---|---|
| Scaffold | g35 activity | g35 activity | g35 activity | g62 activity |
| name | in U2OS | in LCL | in HSC | in U2OS |
| Full f | 98.8% | 45.0% | 15.0% | 96.0% |
| NGS 1 | 7%% | N/A | N/A | N/A |
| NGS 2 | 100% | N/A | N/A | N/A |
| NGS 3 | 100% | N/A | N/A | N/A |
| NGS 6 | 58% | N/A | N/A | N/A |
| NGS 9 | 100% | N/A | N/A | 93% |
| NGS 12 | 100% | 7% | 6% | N/A |
| NGS 13 | 67% | 3% | 6% | N/A |
| NGS 14 | 84% | 5% | 29% | N/A |
| NGS 15 | 91% | 2% | 30% | N/A |
| NGS 16 | 88% | 1% | 14% | N/A |
| NGS 17 | 93% | 53% | 67% | 96% |
| NGS 18 | 84% | N/A | N/A | N/A |
| NGS 40 | N/A | N/A | N/A | N/A |
| NGS 41 | N/A | N/A | N/A | N/A |
| NGS 42 | N/A | N/A | N/A | N/A |
| NGS 43 | N/A | N/A | N/A | N/A |
| NGS 44 | N/A | N/A | N/A | N/A |

Targets for testing activity short-scaffold guide activity

| | Challenging guide | | |
|---|---|---|---|
| Scaffold | g58 activity | g58 activity | g58 activity |
| name | in U2OS | in LCL | in HSC |
| Full f | 95%% | 3.0% | 4.8% |
| NGS_1 | 0% | N/A | N/A |
| NGS_2 | N/A | N/A | N/A |
| NGS_3 | N/A | N/A | N/A |
| NGS_6 | N/A | N/A | N/A |
| NGS_9 | 20% | N/A | N/A |
| NGS_12 | 21% | 0% | 0% |
| NGS_13 | N/A | N/A | N/A |
| NGS_14 | N/A | N/A | N/A |
| NGS_15 | N/A | N/A | N/A |
| NGS_16 | N/A | N/A | N/A |
| NGS_17 | 92% | N/A | 12% |
| NGS_18 | N/A | N/A | N/A |
| NGS_40 | 70% | 1% | 1% |
| NGS_41 | 97% | 10% | 8% |
| NGS_42 | 97% | 16% | 10% |
| NGS_43 | 97% | 24% | 8% |
| NGS_44 | 97% | 20% | 16% |

### REFERENCES

1. Ahmad and Allen (1992) “Antibody-mediated Specific Binging and Cytotoxicity of Lipsome-entrapped Doxorubicin to Lung Cancer Cells in Vitro”, Cancer Research 52:4817-20.
2. Anderson (1992) “Human gene therapy”, Science 256:808-13.
3. Basha et al. (2011) “Influence of Cationic Lipid Composition on Gene Silencing Properties of Lipid Nanoparticle Formulations of siRNA in Antigen-Presenting Cells”, Mol. Ther. 19(12):2186-200.
4. Behr (1994) “Gene transfer with synthetic cationic amphiphiles: Prospects for gene therapy”, Bioconjuage Chem 5:382-89.
5. Blaese et al. (1995) “Vectors in cancer therapy: how will they deliver”, Cancer Gene Ther. 2:291-97.
6. Blaese et al. (1995) “T lympocyte-directed gene therapy for ADA-SCID: initial trial results after 4 years”, Science 270(5235):475-80.
7. Briner et al. (2014) “Guide RNA functional modules direct Cas9 activity and orthognality”, Molecular Cell 56:333-39.
8. Buchschacher and Panganiban (1992) “Human immunodeficiency virus vectors for inducible expression of foreign genes”, J. Virol. 66:2731-39.
9. Burstein et al. (2017) “New CRISPR-Cas systems from uncultivated microbes”, Nature 542:237-41.
10. Canver et al., (2015) “BCL11A enhancer dissection by Cas9-mediated in situ saturating mutagenesis”, Nature Vol. 527, Pgs. 192-214.
11. Chang and Wilson (1987) “Modification of DNA ends can decrease end-joining relative to homologous recombination in mammalian cells”, Proc. Natl. Acad. Sci. USA 84:4959-4963.
12. Charlesworth et al. (2019) “Identification of preexisting adaptive immunity to Cas9 proteins in humans”, Nature Medicine, 25(2), 249.
13. Chung et al. (2006) “Agrobacterium is not alone: gene transfer to plants by viruses and other bacteria”, Trends Plant Sci. 11(1):1-4.
14. Coelho et al. (2013) “Safety and efficacy of RNAi therapy for transthyretin amyloidosis” N. Engl. J. Med. 369, 819-829.
15. Crystal (1995) “Transfer of genes to humans: early lessons and obstacles to success”, Science 270(5235):404-10.
16. Dillon (1993) “Regulation gene expression in gene therapy” Trends in Biotechnology 11(5):167-173.
17. Dranoff et al. (1997) “A phase I study of vaccination with autologous, irradiated melanoma cells engineered to secrete human granulocyte macrophage colony stimulating factor”, Hum. Gene Ther. 8(1):111-23.
18. Dunbar et al. (1995) “Retrovirally marked CD34-enriched peripheral blood and bone marrow cells contribute to long-term engraftment after autologous transplantation”, Blood
19. Ellem et al. (1997) “A case report: immune responses and clinical course of the first human use of ganulocyte/macrophage-colony-stimulating-factor-tranduced autologous melanoma cells for immunotherapy”, Cancer Immunol Immunother 44:10-20.
20. Gao and Huang (1995) “Cationic liposome-mediated gene transfer” Gene Ther. 2(10):710-22.
21. Haddada et al. (1995) “Gene Therapy Using Adenovirus Vectors”, in: The Molecular Repertoire of Adenoviruses III: Biology and Pathogenesis, ed. Doerfler and Bohm, pp. 297-306.
22. Han et al. (1995) “Ligand-directed retro-viral targeting of human breast cancer cells”, Proc. Natl. Acad. Sci. USA 92(21):9747-51.
23. Humbert et al., (2019) “Therapeutically relevant engraftment of a CRISPR-Cas9—edited HSC-enriched population with HbF reactivation in nonhuman primates”, Sci. Trans. Med., Vol. 11, Pgs. 1-13.
24. Inaba et al. (1992) “Generation of large numbers of dendritic cells from mouse bone marrow cultures supplemented with granulocyte/macrophage colony-stimulating factor”, J Exp Med. 176(6):1693-702.
25. Jiang and Doudna (2017) “CRISPR-Cas9 Structures and Mechanisms”, Annual Review of Biophysics 46:505-29.
26. Jinek et al. (2012) “A programmable dual-RNA-guided DNA endonuclease in adaptive bacterial immunity”, Science 337(6096):816-21.
27. Johan et al. (1992) “GLVR1, a receptor for gibbon ape leukemia virus, is homologous to a phosphate permease of Neurospora crassa and is expressed at high levels in the brain and thymus”, J Virol 66(3):1635-40.
28. Judge et al. (2006) “Design of noninflammatory synthetic siRNA mediating potent gene silencing in vivo”, Mol Ther. 13(3):494-505.
29. Kohn et al. (1995) “Engraftment of gene-modified umbilical cord blood cells in neonates with adnosine deaminase deficiency”, Nature Medicine 1:1017-23.
30. Kremer and Perricaudet (1995) “Adenovirus and adeno-associated virus mediated gene transfer”, Br. Med. Bull. 51(1):31-44.
31. Macdiarmid et al. (2009) “Sequential treatment of drug-resistant tumors with targeted minicells containing siRNA or a cytotoxic drug”, Nat Biotehcnol. 27(7):643-51.
32. Malech et al. (1997) “Prolonged production of NADPH oxidase-corrected granulocyes after gene therapy of chronic granulomatous disease”, PNAS 94(22):12133-38.
33. Maxwell et al. (2018) “A detailed cell-free transcription-translation-based assay to decipher CRISPR protospacer adjacent motifs”, Methods 14348-57
34. Miller et al. (1991) “Construction and properties of retrovirus packaging cells based on gibbon ape leukemia virus”, J Virol. 65(5):2220-24.
35. Miller (1992) “Human gene therapy comes of age”, Nature 357:455-60.
36. Mir et al. (2019) “Type II-C CRISPR-Cas9 Biology, Mechanism and Application”, ACS Chem. Biol. 13(2):357-365.
37. Mitani and Caskey (1993) “Delivering therapeutic genes—matching approach and application”, Trends in Biotechnology 11(5):162-66.
38. Nabel and Felgner (1993) “Direct gene transfer for immunotherapy and immunization”, Trends in Biotechnology 11(5):211-15.
39. Nehls et al. (1996) “Two genetically separable steps in the differentiation of thymic epithelium” Science 272:886-889.
40. Nishimasu et al. “Crystal structure of Cas9 in complex with guide RNA and target DNA” (2014) Cell 156(5):935-49.
41. Nishimasu et al. (2015) “Crystal Structure of Staphylococcus aureus Cas9” Cell 162(5):1113-26.
42. Palermo et al. (2018) “Key role of the REC lobe during CRISPR-Cas9 activation by ‘sensing’, ‘regulating’, and ‘locking’ the catalytic HNH domain” Quarterly Reviews of Biophysics 51, e9, 1-11.
43. Remy et al. (1994) “Gene Transfer with a Series of Lipphilic DNA-Binding Molecules”, Bioconjugate Chem. 5(6):647-54.
44. Sentmanat et al. (2018) “A Survey of Validation Strategies for CRISPR-Cas9 Editing”, Scientific Reports 8:888, doi:10.1038/s41598-018-19441-8.

Sommerfelt et al. (1990) “Localization of the receptor gene for type D simian retroviruses on human chromosome 19”, J. Virol. 64(12):6214-20.
46. Van Brunt (1988) “Molecular framing: transgenic animals as bioactors” Biotechnology 6:1149-54.
47. Vigne et al. (1995) “Third-generation adenovectors for gene therapy”, Restorative Neurology and Neuroscience 8(1,2): 35-36.
48. Wagner et al. (2019) “High prevalence of Streptococcus pyogenes Cas9-reactive T cells within the adult human population” Nature Medicine, 25(2), 242
49. Wilson et al. (1989) “Formation of infectious hybrid virion with gibbon ape leukemia virus and human T-cell leukemia virus retroviral envelope glycoproteins and the gag and pol proteins of Moloney murine leukemia virus”, J. Virol. 63:2374-78.
50. Yu et al. (1994) “Progress towards gene therapy for HIV infection”, Gene Ther. 1(1):13-26.
51. Zetsche et al. (2015) “Cpfl is a single RNA-guided endonuclease of a class 2 CRIPSR-Cas system” Cell 163(3):759-71.
52. Zuris et al. (2015) “Cationic lipid-mediated delivery of proteins enables efficient protein based genome editing in vitro and in vivo” Nat Biotechnol. 33(1):73-80.

What is claimed is:

1. A composition comprising a non-naturally occurring RNA molecule, the RNA molecule comprising a crRNA repeat sequence portion and a guide sequence portion, wherein the RNA molecule forms a complex with and targets an OMNI-50 nuclease to a DNA target site in the presence of a tracrRNA sequence, wherein the tracrRNA sequence is encoded by a tracrRNA portion of the RNA molecule or a tracrRNA portion of a second RNA molecule.

2. The composition of claim 1, wherein the crRNA repeat sequence portion is less than 17 nucleotides in length, preferably 12-16 nucleotides in length, or wherein the crRNA repeat sequence portion is 17 or more nucleotides in length, preferably 18-24 nucleotides in length.

3. The composition of claim 1 or 2, wherein the crRNA repeat sequence portion has at least 71-80%, 81-90%, 91-95%, or 96-99% sequence identity to a mature OMNI-50 crRNA repeat sequence encoded by Ezakiella peruensis strain M6.X2.

4. The composition of any one of claims 1-3, wherein the crRNA repeat sequence portion has at least 60-70%, 71-80%, 81-90%, 91-95%, or 96-99% sequence identity to SEQ ID NO: 23.

5. The composition of any one of claims 1-4, wherein the crRNA repeat sequence portion has at least 95% sequence identity to any one of SEQ ID NOs: 8, 23, 24, and 25.

6. The composition of any one of claims 1-5, wherein the crRNA repeat sequence is other than SEQ ID NO: 8 or 23.

7. The composition of any one of claims 1-6, wherein the RNA molecule comprising the crRNA repeat sequence portion and the guide sequence portion further comprises the tracrRNA portion.

8. The composition of claim 7, wherein the crRNA repeat sequence portion is covalently linked to the tracrRNA portion by a polynucleotide linker portion.

9. The composition of any one of claims 1-6, wherein the composition comprises a second RNA molecule comprising the tracrRNA portion.

10. The composition of any one of claims 1-9, wherein the OMNI-50 nuclease has at least 95% sequence identity to the amino acid sequence of SEQ ID NO: 1.

11. The composition of any one of claims 1-10, wherein the guide sequence portion is 17-30 nucleotides in length, preferably 22 nucleotides in length.

12. A composition comprising a non-naturally occurring RNA molecule, the RNA molecule comprising a tracrRNA portion, wherein the RNA molecule forms a complex with and targets an OMNI-50 nuclease to a DNA target site in the presence of a crRNA repeat sequence portion and a guide sequence portion, wherein the crRNA repeat sequence portion and the guide sequence portion are encoded by the RNA molecule or a second RNA molecule.

13. The composition of claim 12, wherein the tracrRNA portion is less than 91 nucleotides in length, preferably 90-80, 89-80, 79-70, 69-60, 59-50, 49-40, or 39-28 nucleotides in length, or wherein the tracrRNA portion is 91 or more nucleotides in length, preferably 91-112 nucleotides in length.

14. The composition of claim 12 or 13, wherein the tracrRNA portion has at least 30-40%, 41-50%, 51-60%, 61-70%, 71-80%, 81-90%, 91-95%, or 96-99% sequence identity to a mature OMNI-50 tracrRNA sequence encoded by Ezakiella peruensis strain M6.X2.

15. The composition of any one of claims 12-14, wherein the tracrRNA portion has at least 30-40%, 41-50%, 51-60%, 61-70%, 71-80%, 81-90%, 91-95%, or 96-99% sequence identity to the tracrRNA portion of SEQ ID NO: 5.

16. The composition of any one of claims 12-15, wherein the tracrRNA portion has at least 95% sequence identity to the tracrRNA portions of any one of SEQ ID NOs: 4, 5, 16-21, 29-31, 74-126, 132-137, and 148-167.

17. The composition of any one of claims 12-16, wherein the tracrRNA portion is other than the tracr portion of SEQ ID NO: 4 or 5.

18. The composition of any one of claims 12-17, wherein the tracrRNA portion comprises a tracrRNA anti-repeat sequence portion that is less than 19 nucleotides in length, preferably 14-18 nucleotides in length.

19. The composition of any one of claims 12-18, wherein the tracrRNA portion comprises a tracrRNA anti-repeat sequence portion that has at least 60-70%, 71-80%, 81-90%, 91-95%, or 96-99% sequence identity to SEQ ID NO: 26.

20. The composition of any one of claims 12-19, wherein the tracrRNA portion comprises a tracrRNA anti-repeat sequence portion that has at least 95% sequence identity to any one of SEQ ID NOs: 9, 26-28, and 138.

21. The composition of any one of claims 12-20, wherein the tracrRNA portion comprises a tracrRNA anti-repeat sequence portion is other than SEQ ID NO: 9 or 26.

22. The composition of any one of claims 12-21, wherein the RNA molecule comprises a tracrRNA portion and further comprises a crRNA repeat sequence portion and a guide sequence portion.

23. The composition of any one of claims 12-22, wherein the tracrRNA portion is covalently linked to the crRNA repeat sequence by a polynucleotide linker portion.

24. The composition of claim 23, wherein the polynucleotide linker portion is 4-10 nucleotides in length.

25. The composition of claim 24, wherein the polynucleotide linker has a sequence of GAAA.

26. The composition of any one of claims 12-21, the composition further comprises a second RNA molecule comprising a crRNA repeat sequence portion and a guide sequence portion.

27. The composition of any one of claims 12-26, wherein the OMNI-50 nuclease is at least 95% sequence identity to the amino acid sequence of SEQ ID NO: 1.

28. The composition of any one of claims 12-27, wherein the guide sequence portion is 17-30 nucleotides in length, preferably 22 nucleotides in length.

29. A composition comprising a non-naturally occurring RNA molecule, the RNA molecule comprising an RNA scaffold portion, the RNA scaffold portion having the structure:
crRNA repeat sequence portion-Linker portion-tracrRNA portion;
wherein the RNA scaffold portion froms a complex with and targets an OMNI-50 CRISPR nuclease to a DNA target site having complimentarity to a guide sequence portion of the RNA molecule.

30. The composition of claim 29, wherein the RNA scaffold portion is 112, 111-110, 109-105, 104-100, 99-95, 94-90, 89-85, 84-80, 79-75, 74-70, 69-50, or 49-45 nucleotides in length.

31. The composition of claim 29 or 30, wherein the RNA scaffold portion has at least 30%, at least 40%, at least 50%, at least 60%, at least 70%, at least 80%, at least 85%, at least 90%, or at least 95% sequence identity to SEQ ID NO: 5.

32. The composition of any one of claims 29-31, wherein the crRNA repeat sequence portion is less than 17 nucleotides in length, preferably 12-16 nucleotides in length, or wherein the crRNA repeat sequence portion is 17 or more nucleotides in length, preferably 18-24 nucleotides in length.

33. The composition any one of claims 29-32, wherein the crRNA repeat sequence portion has at least 60-70%, 71-80%, 81-90%, 91-95%, or 96-99% sequence identity to a mature OMNI-50 crRNA repeat sequence encoded by Ezakiella peruensis strain M6.X2.

34. The composition any one of claims 29-33, wherein the crRNA repeat sequence portion has at least 60-70%, 71-80%, 81-90%, 91-95%, or 96-99% sequence identity to SEQ ID NO:

23. The composition of any one of claims 29-34, wherein the crRNA repeat sequence portion has at least 95% sequence identity to any one of SEQ ID NOs: 8, 23, 24, and 25.

36. The composition of any one of claims 29-35, wherein the crRNA repeat sequence is other than SEQ ID NO: 8 or 23.

37. The composition of any one of claims 29-36, wherein the tracrRNA portion is less than 91 nucleotides in length, preferably 90-80, 89-80, 79-70, 69-60, 59-50, 49-40, or 39-28 nucleotides in length, or wherein the tracrRNA portion is 91 or more nucleotides in length, preferably 91-112 nucleotides in length.

38. The composition of any one of claims 29-37, wherein the tracrRNA portion has at least 30-40%, 41-50%, 51-60%, 61-70%, 71-80%, 81-90%, 91-95%, or 96-99% sequence identity to a mature OMNI-50 tracrRNA sequence encoded by Ezakiella peruensis strain M6.X2.

39. The composition of any one of claims 29-38, wherein the tracrRNA portion has at least 30-40%, 41-50%, 51-60%, 61-70%, 71-80%, 81-90%, 91-95%, or 96-99% sequence identity to the tracrRNA portion of SEQ ID NO: 5.

40. The composition of any one of claims 29-39, wherein the tracrRNA portion has at least 95% sequence identity to the tracrRNA portion of any one of SEQ ID NOs: 4, 5, 16-21, 29-31, 74-126, 132-137, and 148-167.

41. The composition of any one of claims 29-40, wherein the tracrRNA portion is other than the tracrRNA portion of SEQ ID NO: 4 or 5.

42. The composition of any one of claims 29-41, wherein the tracrRNA portion comprises a tracrRNA anti-repeat sequence portion, wherein the crRNA repeat sequence and the tracrRNA anti-repeat sequence portion are covalently linked by the linker portion.

43. The compoisiton of claim 42, wherein the linker portion is a polynucleotide linker that is 4-10 nucleotides in length.

44. The composition of claim 43, the polynucleotide linker has a sequence of GAAA. The composition of any one of claims 42-44, wherein the tracrRNA anti-repeat sequence portion is less than 19 nucleotides in length, preferably 14-18 nucleotides in length.

46. The composition of any one of claims 42-45, wherein the tracrRNA portion comprises a tracrRNA anti-repeat sequence portion that has at least 60-70%, 71-80%, 81-90%, 91-95%, or 96-99% sequence identity to SEQ ID NO: 5.

47. The composition of any one of claims 42-46, wherein the tracrRNA anti-repeat sequence portion has at least 95% sequence identity to any one of SEQ ID NOs: 9, 26-28, and 138.

48. The composition of any one of claims 42-47, wherein the tracrRNA anti-repeat sequence portion is other than SEQ ID NO: 9 or 26.

49. The composition of any one of claims 42-48, wherein the tracrRNA portion comprises a first section of nucleotides linked to the tracrRNA anti-repeat portion, and the first section of nucleotides has at least 95% sequence identity to any one of SEQ ID NOs: 10, 11, 127, 128, 139-143, AAC, A, AA, AAA, and ACAAACC.

50. The composition of any one of claims 42-49, wherein the tracrRNA portion comprises a second section of nucleotides linked to a first section of nucleotides, and the second section of nucleotides has at least 95% sequence identity to any one of SEQ ID NOs: 12, 32, 144-146, GCCUAUU, GCCUAU, AAUGGC, AAAGGC, UAUAGGC, AUAGGC, and GCCU.

51. The composition of any one of claims 42-50, wherein the tracrRNA portion comprises a third section of nucleotides linked to a second section of nucleotides, and the third section of nucleotides has at least 95% sequence identity to any one of SEQ ID NOs: 13, 33, 34, 129, 130, 147, CGCAG, CGC, CGCAGG, C, CUUCUGC, and CGCAGUUG.

52. The composition of any one of claims 42-51, wherein the tracrRNA portion comprises a fourth portion of nucleotides linked to a third section of nucletides, and the fourth section of nucleotides has at least 95% sequence identity to any one of SEQ ID NOs: 14, 15, 35-73, 131, AUU, AUUAUUU, AUUU, AUUUUUUUU, AGCUUUUUU, UUUU, UUUUU, and UUU.

53. The composition of any one of claims 29-52, wherein the RNA scaffold portion has at least 95% identity to the nucleotide sequence of SEQ ID NO: 4, 5, 16-21, 29-31, 74-126, 132-137, and 148-167.

54. The composition of any one of claims 29-53, wherein the RNA scaffold portion has a predicted structure of any one of the Full F, Full C, Short 1, Short 2, Short 3, Short 4, Short Short 6, NGS13, NGS14, NGS15, NGS16, NGS17, NGS18, NGS40, NGS41, NGS42, NGS43, NGS44, NGS9, NGS2, NGS3, NGS12, NGS1, or NGS6 RNA scaffolds.

55. The composition of any one of claims 29-54, wherein the RNA scaffold portion is other than SEQ ID NO: 4 or 5.

56. The composition of any one of claims 29-55, wherein the guide sequence portion is covalently linked to the crRNA repeat sequence portion of the RNA molecule, forming a single-guide RNA molecule having a stucture:
Guide sequence portion-crRNA repeat sequence portion-Linker portion-tracrRNA portion.

57. The composition of any one of claims 29-56, wherein the guide sequence portion is 17-30 nucleotides, more preferably 20-23 nucleotides, more preferably 22 nucleotides in length.

58. The composition of any one of claims 29-57, further comprising an OMNI-50 CRISPR nuclease, wherein the OMNI-50 CRISPR nuclease has at least 95% identity to the amino acid sequence of SEQ ID NO: 1.

59. The composition of any one of claims 29-58, wherein the RNA molecule is formed by in vitro transcription (IVT) or solid-phase artificial oligonucleotide synthesis.

60. The composition of claim 59, wherein the RNA molecule comprises modified nucleotides.

61. A polynucleotide molecule encoding the RNA molecule of any one of claims 29-56.

62. A method of modifying a nucleotide sequence at a DNA target site in a cell-free system or a genome of a cell comprising introducing into the system or cell the composition of any one of claims 29-56 and an OMNI-50 CRISPR nuclease.

63. The method of claim 62, wherein the cell is a eukaryotic cell or a prokaryotic cell.

64. A kit for modifying a nucleotide sequence at a DNA target site in a cell-free system or a genome of a cell comprising introducing into the system or cell the composition of any one of claims 29-56, an OMNI-50 CRISPR nuclease, and instructions for delivering the RNA molecule and the OMNI-50 CRISPR nuclease to the cell.


SEQUENCE LISTING (190 sequences)

SEQ ID NO: 1 - protein, 1370 aa, Ezakiella peruensis strain M6.X2
MTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKL

SEQ ID NO: 2 - DNA, 4113 bp, Ezakiella peruensis strain M6.X2
atgacaaaagtaaaagattattatatcggacttgatataggtacatcatcagttggctgggcagtaacagacgaggcttacaatgttctaaaattcaactccaagaagatgtggggagttcgtctttttgatgatgccaaaactgctgaagaaagacgagggcaaagaggggccaggagaagacttgaccgcaaaaaagaacgcttaagtctcttgcaagatttttttgcagaggaagttgctaaagtagatccaaatttctttttgcgtctagataacagcgacctttatatggaggacaaagatcaaaagttaaagtccaagtacactttatttaatgataaagattttaaagacaagaacttccacaaaaaatatccgactatccaccatctccttatggacttgattgaagatgatagcaaaaaagatattagactggtttatttagcttgccattacttacttaaaaatcgtggccactttatttttgaaggacaaaaatttgatacaaagagctcctttgaaaattctctaaatgaattaaaggtccacttaaatgatgaatacggtcttgatcttgagtttgataatgaaaatttgataaatatacttacagatcctaagttaaacaagaccgcaaaaaagaaagaacttaaaagtgttattggagatacaaaatttctaaaggcagtatctgctattatgattggtagctctcaaaagctagtagatctatttgaaaatcctgaagactttgatgattcggcaatcaaatcagtggatttttctacgacgagttttgatgataaatatagcgattacgagttagcccttggggataaaattgcccttgtaaatatattaaaagaaatctatgactcatctatacttgaaaatttattaaaagaagccgataaatcaaaagatggcaataagtacatttctaacgcctttgtaaaaaaatataacaagcatggccaggacctcaaggaatttaagcgcctagttagacagtaccataaatcagcctacttcgacatctttaggagtgaaaaagtaaacgataactatgtttcatataccaagtcaagtatatccaataacaagagagtgaaggcgaataagtttacagaccaagaagctttttataagtttgctaaaaagcacctagaaactataaaatacaaaattaataaagttaatggtagcaaagctgaccttgaactaatagatggaatgctaagggatatggaatttaaaaatttcatgccaaagataaaatcttctgataatggagttataccttatcaattgaaacttatggagctaaataagatccttgaaaaccaatccaaacaccatgaatttttaaacgtatccgatgaatatggaagcgtttgcgacaagattgcttcgattatggaatttaggattccatattatgttgggcctttaaatcctaactcaaaatatgcttggattaagaagcaaaaggacagcgaaatcacgccatggaattttaaagatgtagttgatttggattcttcaagggaagagtttatagatagcttaattggcaggtgcacatatttaaaagatgaaaaagttctaccaaaggcctcgcttctctacaatgagtatatggttttaaatgaactcaacaatttaaaattaaatgatcttcctattactgaagaaatgaagaagaaaatcttcgatcaactctttaagaccaggaaaaaagtaacattaaaggctgtcgctaatcttctcaaaaaagaatttaatataaatggagaaatcctattgtccggcacagatggggattttaaacaagggctaaactcttataacgattttaaggccattgttggggacaaggttgacagcgacgactatagggataaaatcgaagaaattatcaagctaatcgtcctctatggagatgacaaatcttacttgcaaaagaaaataaaggcgggatacggcaagtattttacagattcagaaatcaaaaagatggctggcctaaattataaagactggggcagattaagtaaaaaactactcacaggtttagaaggcgccaataaaattacaggcgaaagaggatctataatccattttatgcgtgagtacaatttaaacttaatggaattaatgagcgccagcttcacttttacagaggaaattcaaaagttaaatccagttgacgatagaaaactctcctatgagatggttgatgagctttatttatcaccttcagttaagagaatgttatggcaaagtctaagaatagttgatgaaattaaaaatataatgggcactgattccaagaaaatctttattgaaatggccaggggcaaagaagaagtcaaggctagaaaagaatctagaaaaaatcagctcttaaaattttacaaggatggcaaaaaagcctttatatcagaaatcggcgaagaaagatatagctatcttttaagtgaaatcgaaggagaagaggaaaacaaattcagatgggacaatctttatctctactacacccagcttggcaggtgtatgtatagtcttgagccaattgatatttcagaactctcatcgaaaaacatctatgaccaagaccacatttatccaaagtcaaaaatctatgatgattcaattgaaaacagagttttggttaagaaagatttaaatagcaagaaaggcaattcatacccaataccggatgagattttaaataaaaattgctatgcttattggaaaattctatatgacaagggactaattggtcaaaagaaatataccagacttacacgtaggacaggatttactgatgatgaacttgtccaatttatatccaggcaaatagttgagaccaggcaggctaccaaagaaacagcaaatctcttaaaaaccatttgcaaaaattcagaaatagtttactctaaggcagaaaatgctagcagattcagacaggaatttgatatagtaaaatgccgtgcagtcaatgacctccaccacatgcatgacgcttatataaatataatcgttggcaatgtctacaatacaaaatttaccaaagaccccatgaactttgtcaaaaaacaagagaaagctagaagttataacttggaaaacatgtttaaatatgacgtaaagcgcgggggctatacagcatggatagcagacgatgaaaaaggcactgttaaaaatgctagcatcaagagaataagaaaagaactagaggggaccaactacagatttactcgcatgaattatatagaaagtggtgcactatttaatgctaccctgcaaagaaaaaacaaaggaagtcgccctctaaaagataaggggcctaagagctcaatagaaaaatatggtggatatactaatataaacaaggcttgctttgcagtgttggatattaaatcaaaaaataaaatagaaagaaaattaatgccagttgaaagagaaatatacgctaagcaaaagaatgataaaaaattgagtgatgaaatatttagcaaatatttgaaagatagattcggaattgaagattatagagtagtatatcctgtagtaaagatgagaactttgttaaaaatagatggatcttattattttataactggtggaagtgacaaaacattggaattaaggagtgcacttcaattaatattaccaaagaaaaatgaatgggcaataaagcaaattgataaatccagtgagaatgattacctaacaattgaaaggatacaagatttaacggaagaacttgtatacaatacgtttgatataatagtgaataaatttaaaacatctgtatttaaaaaatcatttttgaatttattccaagatgataaaatcgaaaatatagattttaaattcaaatcaatggattttaaagaaaagtgtaaaactctattgatgctagtaaaagccatcagagcttctggtgtacgccaagacttaaaatctatagatttaaaatcagactatggtagattgagctccaagactaataatataggaaactatcaagaatttaaaatcataaaccaatcaattacaggcctctttgaaaacgaagtggacttgttaaaattatga

SEQ ID NO: 3 - DNA, 4107 bp, Artificial Sequence
  Human optimized OMNI-50 DNA sequence
accaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggaccttgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctg

SEQ ID NO: 4 - RNA, 106 nt, Artificial Sequence
  sgRNA V1 scaffold
guuugagaguuauggaaacaugacgaguucaaauaaaaauuuauucaaaccgccuauuuauaggccgcagauguucugcauuaugcuugcuauugcaagcuuuuuu

SEQ ID NO: 5 - RNA, 112 nt, Artificial Sequence
  sgRNA V2 scaffold
guuugagaguuauguaagaaauuacaugacgaguucaaauaaaaauuuauucaaaccgccuauuuauaggccgcagauguucugcauuaugcuugcuauugcaagcuuuuuu

SEQ ID NO: 6 - protein, 1410 aa, Artificial Sequence
  pET9a OMNI50-HisTag Coding ORF Protein Sequence
MPKKKRKVGTTKVKDYYIGLDIGTSSVGWAVTDEAYNVLKFNSKKMWGVRLFDDAKTAEERRGQRGARRRLDRKKERLSLLQDFFAEEVAKVDPNFFLRLDNSDLYMEDKDQKLKSKYTLFNDKDFKDKNFHKKYPTIHHLLMDLIEDDSKKDIRLVYLACHYLLKNRGHFIFEGQKFDTKSSFENSLNELKVHLNDEYGLDLEFDNENLINILTDPKLNKTAKKKELKSVIGDTKFLKAVSAIMIGSSQKLVDLFENPEDFDDSAIKSVDFSTTSFDDKYSDYELALGDKIALVNILKEIYDSSILENLLKEADKSKDGNKYISNAFVKKYNKHGQDLKEFKRLVRQYHKSAYFDIFRSEKVNDNYVSYTKSSISNNKRVKANKFTDQEAFYKFAKKHLETIKYKINKVNGSKADLELIDGMLRDMEFKNFMPKIKSSDNGVIPYQLKLMELNKILENQSKHHEFLNVSDEYGSVCDKIASIMEFRIPYYVGPLNPNSKYAWIKKQKDSEITPWNFKDVVDLDSSREEFIDSLIGRCTYLKDEKVLPKASLLYNEYMVLNELNNLKLNDLPITEEMKKKIFDQLFKTRKKVTLKAVANLLKKEFNINGEILLSGTDGDFKQGLNSYNDFKAIVGDKVDSDDYRDKIEEIIKLIVLYGDDKSYLQKKIKAGYGKYFTDSEIKKMAGLNYKDWGRLSKKLLTGLEGANKITGERGSIIHFMREYNLNLMELMSASFTFTEEIQKLNPVDDRKLSYEMVDELYLSPSVKRMLWQSLRIVDEIKNIMGTDSKKIFIEMARGKEEVKARKESRKNQLLKFYKDGKKAFISEIGEERYSYLLSEIEGEEENKFRWDNLYLYYTQLGRCMYSLEPIDISELSSKNIYDQDHIYPKSKIYDDSIENRVLVKKDLNSKKGNSYPIPDEILNKNCYAYWKILYDKGLIGQKKYTRLTRRTGFTDDELVQFISRQIVETRQATKETANLLKTICKNSEIVYSKAENASRFRQEFDIVKCRAVNDLHHMHDAYINIIVGNVYNTKFTKDPMNFVKKQEKARSYNLENMFKYDVKRGGYTAWIADDEKGTVKNASIKRIRKELEGTNYRFTRMNYIESGALFNATLQRKNKGSRPLKDKGPKSSIEKYGGYTNINKACFAVLDIKSKNKIERKLMPVEREIYAKQKNDKKLSDEIFSKYLKDRFGIEDYRVVYPVVKMRTLLKIDGSYYFITGGSDKTLELRSALQLILPKKNEWAIKQIDKSSENDYLTIERIQDLTEELVYNTFDIIVNKFKTSVFKKSFLNLFQDDKIENIDFKFKSMDFKEKCKTLLMLVKAIRASGVRQDLKSIDLKSDYGRLSSKTNNIGNYQEFKIINQSITGLFENEVDLLKLGSYPYDVPDYAAAPKKKRKVAASHHHHHHHH

SEQ ID NO: 7 - DNA, 6439 bp, Artificial Sequence
  pET9a OMNI50-HisTag DNA sequence
taatacgactcactatagggagaccacaacggtttccctctagagagacaataaccctgataatgcttcaataatattgaaaaaggaagagtatgcctaagaagaagagaaaggtgggtaccaccaaggtgaaggactactacataggcttggacatcggcacctctagcgtcgggtgggccgtcaccgatgaagcctataacgtgcttaagtttaatagcaagaaaatgtggggcgtgcggctgttcgacgacgctaagacggcagaggagcgtaggggccagcgaggagcaagacgacgtctggatcggaagaaggagagactcagcctgctgcaggacttcttcgccgaagaggtagcaaaggtcgaccccaacttcttcctcaggctggacaattccgatctgtacatggaagataaggaccagaaactgaaaagcaaatatacactgttcaacgacaaggacttcaaggataagaattttcataagaagtaccccacaatacatcacctgctgatggatctgatcgaggacgacagtaagaaggacatccggctcgtctacctggcctgtcactatttgctcaagaacaggggtcatttcatcttcgagggccagaagttcgacactaaatcaagcttcgagaacagtttgaacgagctcaaagttcatttgaacgacgagtatggactggacctcgaatttgacaacgagaacctgattaacatcttgactgacccaaaactcaataaaacggccaagaagaaggagctgaagtccgtaatcggcgacaccaagttcctcaaagccgtttccgcgataatgatcggctctagccagaaactcgtcgacttgttcgagaaccccgaggatttcgacgactctgcgataaagtccgttgacttctcaactacctctttcgacgacaagtactctgactatgaactcgctctgggtgacaagatcgctctggtcaacatccttaaggaaatttacgatagctccatcctcgagaacctgctcaaagaggcagacaagtctaaggacggtaacaaatatatcagtaatgcattcgtgaagaagtacaataaacacggacaagatctgaaagagttcaaacgtctggtacgacaatatcacaagagtgcgtattttgatattttcagatccgagaaggtgaatgacaattacgtcagctacactaaaagctcaattagcaacaataaacgcgtcaaagcaaacaagttcactgatcaagaggccttctacaaattcgccaagaaacatctggagacaatcaagtataagatcaacaaggtaaacggctccaaggcagatctggagctgattgacgggatgctgcgggacatggagttcaagaactttatgcccaaaattaagtccagtgacaacggggtgattccataccagctcaagctgatggaattgaacaaaatactcgagaatcagtcaaagcatcacgagttcctcaatgtcagcgacgagtacggctccgtgtgtgataaaatcgcatctatcatggagttccgtatcccctactacgtgggacccctgaaccccaatagcaagtacgcctggatcaagaagcagaaagatagtgagattactccctggaacttcaaggacgtcgtggacctcgactccagcagagaggagttcattgactcactgatcggacgctgtacttaccttaaggacgagaaggtccttcccaaagcttctttgctgtataacgaatacatggtgctgaacgagctgaataacctgaagttgaacgaccttcccatcaccgaggagatgaagaagaagatatttgaccagttgttcaaaacaagaaagaaggtcacccttaaagcggtggcaaacctgctgaagaaggagttcaacatcaacggcgagattctgctctctgggaccgacggtgacttcaagcagggcttgaactcatacaatgacttcaaagctatcgtgggcgataaagtcgattccgatgattaccgggacaagattgaggagatcattaaactgatagttctttacggtgacgataagagttaccttcagaagaagattaaagctgggtatggaaaatacttcaccgacagtgagattaagaaaatggcggggctgaactacaaggattggggaaggctctcaaagaagctgctgacgggactcgagggtgcaaacaagatcactggagagcggggctccattattcacttcatgagggaatataaccttaatctgatggagcttatgtcagcttcatttacgttcaccgaagagatacagaaacttaaccccgtggatgaccgcaagctgtcatacgaaatggtggacgaactgtacctttctcccagtgtgaaacggatgctctggcagtccctgcgcatcgtcgacgagataaagaacatcatgggaaccgacagtaagaagattttcatcgagatggctcggggtaaggaagaggtgaaagcccgcaaggagtcaaggaagaaccaactgctgaagttctataaagacggaaagaaggcattcatcagcgagattggcgaggagaggtactcttacttgctttctgagatagagggtgaggaagagaataagtttcgatgggataacctgtacctttattatactcaactgggtcgctgcatgtactctttggaacctatcgacatatctgagctgtcttcaaagaatatttacgatcaggatcatatctaccccaaaagcaagatttacgacgacagtatcgagaatagggtgctggtgaagaaggaccttaactccaagaagggtaacagctatcctatcccagacgaaatcctgaacaagaactgttacgcctactggaagatcctgtacgataaaggtcttatcgggcagaagaagtacactcggctgacccggagaactggcttcacggacgacgagctcgttcagttcatctcaagacagatcgtggaaactagacaagcaacaaaggagactgctaacctgctcaagacaatatgtaagaactccgagatcgtgtattccaaagccgagaacgcaagtcggtttaggcaagagttcgacatcgtgaagtgtagggcggtgaacgatcttcatcatatgcacgatgcctacatcaacatcatagtggggaacgtgtataacaccaagttcacgaaggaccctatgaatttcgtaaagaagcaggaaaaggcgcggagctacaatctcgagaatatgttcaagtacgatgtgaaacgtggcggatacaccgcttggatcgccgatgacgagaagggcaccgtgaagaacgcgagtattaaacgtatccggaaggagctggaaggcacaaattataggttcacaagaatgaactacattgagtctggagcgcttttcaacgccactctccagcggaagaataagggctccagacccctgaaggacaaaggcccgaaatcttccatcgagaagtacggcggctacacaaacatcaataaagcctgtttcgctgttcttgacatcaagtctaagaacaagattgagaggaagctgatgcccgtcgagcgtgagatctatgccaaacagaagaacgacaagaagctgtccgacgagattttctcaaagtacctcaaggaccgatttggcatcgaggactacagggttgtctacccagtggtgaaaatgcgcacactgctcaagatcgacggcagctactacttcatcacaggcggttctgataagaccctggagttgcgatctgctctgcagctgattctccctaagaagaacgagtgggcgatcaaacagatcgacaagtcttccgaaaacgactatctgacgatcgagcgtatccaggacctgaccgaggagctggtgtataacactttcgacatcatcgtcaacaagttcaagaccagtgtcttcaagaagtctttccttaacttgtttcaggacgacaagattgagaacattgacttcaagtttaagtccatggacttcaaggagaaatgcaagacacttctcatgctggtcaaggcgattcgggcatccggcgtgaggcaggatctcaagtccatcgacctcaagtctgattacggacggctcagttcaaagaccaacaacatcggcaattaccaggagttcaagattattaatcagtccatcactggactgttcgagaatgaggtcgatctcctgaagctgggatcctacccatacgatgttccagattacgcggccgctccaaaaaagaaaagaaaagttgcggctagccatcatcaccatcaccatcatcattaaggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcgatcccgcgaaat

SEQ ID NO: 8 - RNA, 14 nt, Artificial Sequence
  OMNI-50 scaffold or scaffold portion
guuugagaguuaug

SEQ ID NO: 9 - RNA, 16 nt, Artificial Sequence
  OMNI-50 scaffold or scaffold portion
caugacgaguucaaau

SEQ ID NO: 10 - RNA, 17 nt, Artificial Sequence
  OMNI-50 scaffold or scaffold portion
aaaaauuuauucaaacc

SEQ ID NO: 11 - RNA, 12 nt, Artificial Sequence
  OMNI-50 scaffold or scaffold portion
aaaaauuuauuc

SEQ ID NO: 12 - RNA, 14 nt, Artificial Sequence
  OMNI-50 scaffold or scaffold portion
gccuauuuauaggc

SEQ ID NO: 13 - RNA, 14 nt, Artificial Sequence
  OMNI-50 scaffold or scaffold portion
cgcagauguucugc

SEQ ID NO: 14 - RNA, 27 nt, Artificial Sequence
  OMNI-50 scaffold or scaffold portion
auuaugcuugcuauugcaagcuuuuuu

SEQ ID NO: 15 - RNA, 15 nt, Artificial Sequence
  OMNI-50 scaffold or scaffold portion
auuaugcuugcuauu

SEQ ID NO: 16 - RNA, 94 nt, Artificial Sequence
  OMNI-50 scaffold or scaffold portion
guuugagaguuauggaaacaugacgaguucaaauaaaaauuuauucaaaccgccuauuuauaggccgcagauguucugcauuaugcuugcuauu

SEQ ID NO: 17 - RNA, 82 nt, Artificial Sequence
  OMNI-50 scaffold or scaffold portion
guuugagaguuauggaaacaugacgaguucaaauaaaaauuuauucaaaccgccuauuuauaggccgcagauguucugcauu

SEQ ID NO: 18 - RNA, 70 nt, Artificial Sequence
  OMNI-50 scaffold or scaffold portion
guuugagaguuauggaaacaugacgaguucaaauaaaaauuuauucaaaccgccuauuuauaggccgcag

SEQ ID NO: 19 - RNA, 58 nt, Artificial Sequence
  OMNI-50 scaffold or scaffold portion
guuugagaguuauggaaacaugacgaguucaaauaaaaauuuauucaaaccgccuauu

SEQ ID NO: 20 - RNA, 46 nt, Artificial Sequence
  OMNI-50 scaffold or scaffold portion
guuugagaguuauggaaacaugacgaguucaaauaaaaauuuauuc

SEQ ID NO: 21 - RNA, 34 nt, Artificial Sequence
  OMNI-50 scaffold or scaffold portion
guuugagaguuauggaaacaugacgaguucaaau

SEQ ID NO: 22 - RNA, 54 nt, Artificial Sequence
  OMNI-50 scaffold or scaffold portion
guuugagaguuauguaauuucauauaggacuaaaacgaaagcuauaauauguau

SEQ ID NO: 23 - RNA, 17 nt, Artificial Sequence
  OMNI-50 scaffold or scaffold portion
guuugagaguuauguaa

SEQ ID NO: 24 - RNA, 13 nt, Artificial Sequence
  OMNI-50 scaffold or scaffold portion
guuugagaguuau

SEQ ID NO: 25 - RNA, 12 nt, Artificial Sequence
  OMNI-50 scaffold or scaffold portion
guuugagaguua

SEQ ID NO: 26 - RNA, 19 nt, Artificial Sequence
  OMNI-50 scaffold or scaffold portion
uuacaugacgaguucaaau

SEQ ID NO: 27 - RNA, 15 nt, Artificial Sequence
  OMNI-50 scaffold or scaffold portion
augacgaguucaaau

SEQ ID NO: 28 - RNA, 14 nt, Artificial Sequence
  OMNI-50 scaffold or scaffold portion
ugacgaguucaaau

SEQ ID NO: 29 - RNA, 149 nt, Artificial Sequence
  OMNI-50 scaffold or scaffold portion
guuugagaguuauguaauuucauauaggacuaaaacgaaagcuauaauauguaugaaauuacaugacgaguucaaauaaaaauuuauucaaaccgccuauuuauaggccgcagauguucugcauuaugcuugcuauugcaagcuuuuuu

SEQ ID NO: 30 - RNA, 104 nt, Artificial Sequence
  OMNI-50 scaffold or scaffold portion
guuugagaguuaugaaaaugacgaguucaaauaaaaauuuauucaaaccgccuauuuauaggccgcagauguucugcauuaugcuugcuauugcaagcuuuuuu

SEQ ID NO: 31 - RNA, 102 nt, Artificial Sequence
  OMNI-50 scaffold or scaffold portion
guuugagaguuagaaaugacgaguucaaauaaaaauuuauucaaaccgccuauuuauaggccgcagauguucugcauuaugcuugcuauugcaagcuuuuuu

SEQ ID NO: 32 - RNA, 10 nt, Artificial Sequence
  OMNI-50 scaffold or scaffold portion
gccuauaggc

SEQ ID NO: 33 - RNA, 14 nt, Artificial Sequence
  OMNI-50 scaffold or scaffold portion
cgcagauguucugu

SEQ ID NO: 34 - RNA, 13 nt, Artificial Sequence
  OMNI-50 scaffold or scaffold portion
cgcagauguucug

SEQ ID NO: 35 - RNA, 19 nt, Artificial Sequence
  OMNI-50 scaffold or scaffold portion
agcuugcuauugcaauuuu

SEQ ID NO: 36 - RNA, 11 nt, Artificial Sequence
  OMNI-50 scaffold or scaffold portion
auuauuuuuuu

SEQ ID NO: 37 - RNA, 19 nt, Artificial Sequence
  OMNI-50 scaffold or scaffold portion
auuaugcuuuugcaauuuu

SEQ ID NO: 38 - RNA, 17 nt, Artificial Sequence
  OMNI-50 scaffold or scaffold portion
auuuugcaagcuuuuuu

SEQ ID NO: 39 - RNA, 17 nt, Artificial Sequence
  OMNI-50 scaffold or scaffold portion
cuauugcaagcuuuuuu

SEQ ID NO: 40 - RNA, 11 nt, Artificial Sequence
  OMNI-50 scaffold or scaffold portion
caagcuuuuuu

SEQ ID NO: 41 - RNA, 19 nt, Artificial Sequence
  OMNI-50 scaffold or scaffold portion
auuaugcuauugcauuuuu

SEQ ID NO: 42 - RNA, 16 nt, Artificial Sequence
  OMNI-50 scaffold or scaffold portion
uauugcaagcuuuuuu

SEQ ID NO: 43 - RNA, 11 nt, Artificial Sequence
  OMNI-50 scaffold or scaffold portion
aaagcuuuuuu

SEQ ID NO: 44 - RNA, 19 nt, Artificial Sequence
  OMNI-50 scaffold or scaffold portion
auuuugcuauugcuuuuuu

SEQ ID NO: 45 - RNA, 15 nt, Artificial Sequence
  OMNI-50 scaffold or scaffold portion
auugcaagcuuuuuu

SEQ ID NO: 46 - RNA, 13 nt, Artificial Sequence
  OMNI-50 scaffold or scaffold portion
agcaagcuuuuuu

SEQ ID NO: 47 - RNA, 19 nt, Artificial Sequence
  OMNI-50 scaffold or scaffold portion
auuaauugcaagcuuuuuu

SEQ ID NO: 48 - RNA, 19 nt, Artificial Sequence
  OMNI-50 scaffold or scaffold portion
auuaugcgcaagcuuuuuu

SEQ ID NO: 49 - RNA, 11 nt, Artificial Sequence
  OMNI-50 scaffold or scaffold portion
auagcuuuuuu

SEQ ID NO: 50 - RNA, 21 nt, Artificial Sequence
  OMNI-50 scaffold or scaffold portion
auuacuauugcaagcuuuuuu

SEQ ID NO: 51 - RNA, 15 nt, Artificial Sequence
  OMNI-50 scaffold or scaffold portion
auuacaagcuuuuuu

SEQ ID NO: 52 - RNA, 21 nt, Artificial Sequence
  OMNI-50 scaffold or scaffold portion
auugcuauugcaagcuuuuuu

SEQ ID NO: 53 - RNA, 19 nt, Artificial Sequence
  OMNI-50 scaffold or scaffold portion
ugcuugcuauagcuuuuuu

SEQ ID NO: 54 - RNA, 14 nt, Artificial Sequence
  OMNI-50 scaffold or scaffold portion
uugcaagcuuuuuu

SEQ ID NO: 55 - RNA, 19 nt, Artificial Sequence
  OMNI-50 scaffold or scaffold portion
aucuauugcaagcuuuuuu

SEQ ID NO: 56 - RNA, 23 nt, Artificial Sequence
  OMNI-50 scaffold or scaffold portion
aucuugcuauugcaagcuuuuuu

SEQ ID NO: 57 - RNA, 13 nt, Artificial Sequence
  OMNI-50 scaffold or scaffold portion
aucaagcuuuuuu

SEQ ID NO: 58 - RNA, 15 nt, Artificial Sequence
  OMNI-50 scaffold or scaffold portion
auuaugcucuuuuuu

SEQ ID NO: 59 - RNA, 17 nt, Artificial Sequence
  OMNI-50 scaffold or scaffold portion
auauugcaagcuuuuuu

SEQ ID NO: 60 - RNA, 13 nt, Artificial Sequence
  OMNI-50 scaffold or scaffold portion
auuaugcuuuuuu

SEQ ID NO: 61 - RNA, 21 nt, Artificial Sequence
  OMNI-50 scaffold or scaffold portion
auuaugcuugcuauagcuuuu

SEQ ID NO: 62 - RNA, 19 nt, Artificial Sequence
  OMNI-50 scaffold or scaffold portion
ugcuugcuauugcaauuuu

SEQ ID NO: 63 - RNA, 15 nt, Artificial Sequence
  OMNI-50 scaffold or scaffold portion
auuauaagcuuuuuu

SEQ ID NO: 64 - RNA, 11 nt, Artificial Sequence
  OMNI-50 scaffold or scaffold portion
auuacuuuuuu

SEQ ID NO: 65 - RNA, 13 nt, Artificial Sequence
  OMNI-50 scaffold or scaffold portion
auuaagcuuuuuu

SEQ ID NO: 66 - RNA, 21 nt, Artificial Sequence
  OMNI-50 scaffold or scaffold portion
auuauuauugcaagcuuuuuu

SEQ ID NO: 67 - RNA, 19 nt, Artificial Sequence
  OMNI-50 scaffold or scaffold portion
auuuauugcaagcuuuuuu

SEQ ID NO: 68 - RNA, 19 nt, Artificial Sequence
  OMNI-50 scaffold or scaffold portion
ugcuugcucaagcuuuuuu

SEQ ID NO: 69 - RNA, 19 nt, Artificial Sequence
  OMNI-50 scaffold or scaffold portion
auuuuuugcaagcuuuuuu

SEQ ID NO: 70 - RNA, 19 nt, Artificial Sequence
  OMNI-50 scaffold or scaffold portion
ugcuuuugcaagcuuuuuu

SEQ ID NO: 71 - RNA, 19 nt, Artificial Sequence
  OMNI-50 scaffold or scaffold portion
auuuugugcaagcuuuuuu

SEQ ID NO: 72 - RNA, 19 nt, Artificial Sequence
  OMNI-50 scaffold or scaffold portion
auuaugcuuaagcuuuuuu

SEQ ID NO: 73 - RNA, 19 nt, Artificial Sequence
  OMNI-50 scaffold or scaffold portion
ugcuauugcaagcuuuuuu

SEQ ID NO: 74 - RNA, 96 nt, Artificial Sequence
  OMNI-50 scaffold or scaffold portion
guuugagaguuaugaaaaugacgaguucaaauaaaaauuuauucaaaccgccuauuuauaggccgcagauguucugcagcuugcuauugcaauuuu

SEQ ID NO: 75 - RNA, 86 nt, Artificial Sequence
  OMNI-50 scaffold or scaffold portion
guuugagaguuagaaaugacgaguucaaauaaaaauuuauucaaaccgccuauuuauaggccgcagauguucugcauuauuuuuuu

SEQ ID NO: 76 - RNA, 98 nt, Artificial Sequence
  OMNI-50 scaffold or scaffold portion
guuugagaguuauggaaacaugacgaguucaaauaaaaauuuauucaaaccgccuauuuauaggccgcagauguucugcauuaugcuuuugcaauuuu

SEQ ID NO: 77 - RNA, 96 nt, Artificial Sequence
  OMNI-50 scaffold or scaffold portion
guuugagaguuauggaaacaugacgaguucaaauaaaaauuuauucaaaccgccuauuuauaggccgcagauguucugcauuuugcaagcuuuuuu

SEQ ID NO: 78 - RNA, 96 nt, Artificial Sequence
  OMNI-50 scaffold or scaffold portion
guuugagaguuauggaaacaugacgaguucaaauaaaaauuuauucaaaccgccuauuuauaggccgcagauguucugccuauugcaagcuuuuuu

SEQ ID NO: 79 - RNA, 90 nt, Artificial Sequence
  OMNI-50 scaffold or scaffold portion
guuugagaguuauggaaacaugacgaguucaaauaaaaauuuauucaaaccgccuauuuauaggccgcagauguucugccaagcuuuuuu

SEQ ID NO: 80 - RNA, 98 nt, Artificial Sequence
  OMNI-50 scaffold or scaffold portion
guuugagaguuauggaaacaugacgaguucaaauaaaaauuuauucaaaccgccuauuuauaggccgcagauguucugcauuaugcuauugcauuuuu

SEQ ID NO: 81 - RNA, 84 nt, Artificial Sequence
  OMNI-50 scaffold or scaffold portion
guuugagaguuauggaaacaugacgaguucaaauaaaaauuuauucaaaccgccuauuuauaggccgcuauugcaagcuuuuuu

SEQ ID NO: 82 - RNA, 82 nt, Artificial Sequence
  OMNI-50 scaffold or scaffold portion
guuugagaguuagaaaugacgaguucaaauaaaaauuuauucaaaccgccuauuuauaggccgcagauguucugcauuauuu

SEQ ID NO: 83 - RNA, 79 nt, Artificial Sequence
  OMNI-50 scaffold or scaffold portion
guuugagaguuagaaaugacgaguucaaauaaaaauuuauucaaaccgccuauuuauaggccgcagauguucugcauuu

SEQ ID NO: 84 - RNA, 87 nt, Artificial Sequence
  OMNI-50 scaffold or scaffold portion
guuugagaguuaugaaaugacgaguucaaauaaaaauuuauucaaaccgccuauuuauaggccgcagauguucugcaaagcuuuuuu

SEQ ID NO: 85 - RNA, 98 nt, Artificial Sequence
  OMNI-50 scaffold or scaffold portion
guuugagaguuauggaaacaugacgaguucaaauaaaaauuuauucaaaccgccuauuuauaggccgcagauguucugcauuuugcuauugcuuuuuu

SEQ ID NO: 86 - RNA, 98 nt, Artificial Sequence
  OMNI-50 scaffold or scaffold portion
guuugagaguuauggaaacaugacgaguucaaauaaaaauuuauucaaaccgccuauuuauaggccgcagauguucugcagcuugcuauugcaauuuu

SEQ ID NO: 87 - RNA, 94 nt, Artificial Sequence
  OMNI-50 scaffold or scaffold portion
guuugagaguuauggaaacaugacgaguucaaauaaaaauuuauucaaaccgccuauuuauaggccgcagauguucuguauugcaagcuuuuuu

SEQ ID NO: 88 - RNA, 92 nt, Artificial Sequence
  OMNI-50 scaffold or scaffold portion
guuugagaguuauggaaacaugacgaguucaaauaaaaauuuauucaaaccgccuauuuauaggccgcagauguucugcagcaagcuuuuuu

SEQ ID NO: 89 - RNA, 98 nt, Artificial Sequence
  OMNI-50 scaffold or scaffold portion
guuugagaguuauggaaacaugacgaguucaaauaaaaauuuauucaaaccgccuauuuauaggccgcagauguucugcauuaauugcaagcuuuuuu

SEQ ID NO: 90 - RNA, 94 nt, Artificial Sequence
  OMNI-50 scaffold or scaffold portion
guuugagaguuagaaaugacgaguucaaauaaaaauuuauucaaaccgccuauuuauaggccgcagauguucugcauuaugcgcaagcuuuuuu

SEQ ID NO: 91 - RNA, 90 nt, Artificial Sequence
  OMNI-50 scaffold or scaffold portion
guuugagaguuauggaaacaugacgaguucaaauaaaaauuuauucaaaccgccuauuuauaggccgcagauguucugcauagcuuuuuu

SEQ ID NO: 92 - RNA, 100 nt, Artificial Sequence
  OMNI-50 scaffold or scaffold portion
guuugagaguuauggaaacaugacgaguucaaauaaaaauuuauucaaaccgccuauuuauaggccgcagauguucugcauuacuauugcaagcuuuuuu

SEQ ID NO: 93 - RNA, 94 nt, Artificial Sequence
  OMNI-50 scaffold or scaffold portion
guuugagaguuauggaaacaugacgaguucaaauaaaaauuuauucaaaccgccuauuuauaggccgcagauguucugcauuacaagcuuuuuu

SEQ ID NO: 94 - RNA, 100 nt, Artificial Sequence
  OMNI-50 scaffold or scaffold portion
guuugagaguuauggaaacaugacgaguucaaauaaaaauuuauucaaaccgccuauuuauaggccgcagauguucugcauugcuauugcaagcuuuuuu

SEQ ID NO: 95 - RNA, 90 nt, Artificial Sequence
  OMNI-50 scaffold or scaffold portion
guuugagaguuauggaaacaugacgaguucaaauaaaaauuuauucaaaccgccuauuuauaggccgcagauguucugcauuauuuuuuu

SEQ ID NO: 96 - RNA, 98 nt, Artificial Sequence
  OMNI-50 scaffold or scaffold portion
guuugagaguuauggaaacaugacgaguucaaauaaaaauuuauucaaaccgccuauuuauaggccgcagauguucugcugcuugcuauagcuuuuuu

SEQ ID NO: 97 - RNA, 92 nt, Artificial Sequence
  OMNI-50 scaffold or scaffold portion
guuugagaguuauggaaacaugacgaguucaaauaaaaauuuauucaaaccgccuauuuauaggccgcagauguucuguugcaagcuuuuuu

SEQ ID NO: 98 - RNA, 98 nt, Artificial Sequence
  OMNI-50 scaffold or scaffold portion
guuugagaguuauggaaacaugacgaguucaaauaaaaauuuauucaaaccgccuauuuauaggccgcagauguucugcaucuauugcaagcuuuuuu

SEQ ID NO: 99 - RNA, 98 nt, Artificial Sequence
  OMNI-50 scaffold or scaffold portion
guuugagaguuauggaaacaugacgaguucaaauaaaaauuuauucaaaccgccuauaggccgcagauguucugcaucuugcuauugcaagcuuuuuu

SEQ ID NO: 100 - RNA, 89 nt, Artificial Sequence
  OMNI-50 scaffold or scaffold portion
guuugagaguuaugaaaugacgaguucaaauaaaaauuuauucaaaccgccuauuuauaggccgcagauguucugcaucaagcuuuuuu

SEQ ID NO: 101 - RNA, 94 nt, Artificial Sequence
  OMNI-50 scaffold or scaffold portion
guuugagaguuauggaaacaugacgaguucaaauaaaaauuuauucaaaccgccuauuuauaggccgcagauguucugcauuaugcucuuuuuu

SEQ ID NO: 102 - RNA, 92 nt, Artificial Sequence
  OMNI-50 scaffold or scaffold portion
guuugagaguuauggaaacaugacgaguucaaauaaaaauuuauucaaaccgccuauuuauaggccgcagauguucugcaucaagcuuuuuu

SEQ ID NO: 103 - RNA, 96 nt, Artificial Sequence
  OMNI-50 scaffold or scaffold portion
guuugagaguuauggaaacaugacgaguucaaauaaaaauuuauucaaaccgccuauuuauaggccgcagauguucugcauauugcaagcuuuuuu

SEQ ID NO: 104 - RNA, 90 nt, Artificial Sequence
  OMNI-50 scaffold or scaffold portion
guuugagaguuauggaaacaugacgaguucaaauaaaaauuuauucaaaccgccuauuuauaggccgcagauguucugcaaagcuuuuuu

SEQ ID NO: 105 - RNA, 92 nt, Artificial Sequence
  OMNI-50 scaffold or scaffold portion
guuugagaguuauggaaacaugacgaguucaaauaaaaauuuauucaaaccgccuauuuauaggccgcagauguucugcauuaugcuuuuuu

SEQ ID NO: 106 - RNA, 84 nt, Artificial Sequence
  OMNI-50 scaffold or scaffold portion
guuugagaguuauggaaacaugacgaguucaaauaaaaauuuauucaaaccgccuauuuauaggccgcagauguucugcauuau

SEQ ID NO: 107 - RNA, 100 nt, Artificial Sequence
  OMNI-50 scaffold or scaffold portion
guuugagaguuauggaaacaugacgaguucaaauaaaaauuuauucaaaccgccuauuuauaggccgcagauguucugcauuaugcuugcuauagcuuuu

SEQ ID NO: 108 - RNA, 98 nt, Artificial Sequence
  OMNI-50 scaffold or scaffold portion
guuugagaguuauggaaacaugacgaguucaaauaaaaauuuauucaaaccgccuauuuauaggccgcagauguucugcugcuugcuauugcaauuuu

SEQ ID NO: 109 - RNA, 94 nt, Artificial Sequence
  OMNI-50 scaffold or scaffold portion
guuugagaguuauggaaacaugacgaguucaaauaaaaauuuauucaaaccgccuauuuauaggccgcagauguucugcauuauaagcuuuuuu

SEQ ID NO: 110 - RNA, 90 nt, Artificial Sequence
  OMNI-50 scaffold or scaffold portion
guuugagaguuauggaaacaugacgaguucaaauaaaaauuuauucaaaccgccuauuuauaggccgcagauguucugcauuacuuuuuu

SEQ ID NO: 111 - RNA, 88 nt, Artificial Sequence
  OMNI-50 scaffold or scaffold portion
guuugagaguuauggaaacaugacgaguucaaauaaaaauuuauucaaaccgccuauuuauaggccgcagauguucugcauuuuuuuu

SEQ ID NO: 112 - RNA, 92 nt, Artificial Sequence
  OMNI-50 scaffold or scaffold portion
guuugagaguuauggaaacaugacgaguucaaauaaaaauuuauucaaaccgccuauuuauaggccgcagauguucugcauuaagcuuuuuu

SEQ ID NO: 113 - RNA, 90 nt, Artificial Sequence
  OMNI-50 scaffold or scaffold portion
guuugagaguuagaaaugacgaguucaaauaaaaauuuauucaaaccgccuauuuauaggccgcagauguucugcauugcaagcuuuuuu

SEQ ID NO: 114 - RNA, 100 nt, Artificial Sequence
  OMNI-50 scaffold or scaffold portion
guuugagaguuauggaaacaugacgaguucaaauaaaaauuuauucaaaccgccuauuuauaggccgcagauguucugcauuauuauugcaagcuuuuuu

SEQ ID NO: 115 - RNA, 98 nt, Artificial Sequence
  OMNI-50 scaffold or scaffold portion
guuugagaguuauggaaacaugacgaguucaaauaaaaauuuauucaaaccgccuauuuauaggccgcagauguucugcauuuauugcaagcuuuuuu

SEQ ID NO: 116 - RNA, 98 nt, Artificial Sequence
  OMNI-50 scaffold or scaffold portion
guuugagaguuauggaaacaugacgaguucaaauaaaaauuuauucaaaccgccuauuuauaggccgcagauguucugcugcuugcucaagcuuuuuu

SEQ ID NO: 117 - RNA, 98 nt, Artificial Sequence
  OMNI-50 scaffold or scaffold portion
guuugagaguuauggaaacaugacgaguucaaauaaaaauuuauucaaaccgccuauuuauaggccgcagauguucugcauuuuuugcaagcuuuuuu

SEQ ID NO: 118 - RNA, 98 nt, Artificial Sequence
  OMNI-50 scaffold or scaffold portion
guuugagaguuauggaaacaugacgaguucaaauaaaaauuuauucaaaccgccuauuuauaggccgcagauguucugcugcuuuugcaagcuuuuuu

SEQ ID NO: 119 - RNA, 98 nt, Artificial Sequence
  OMNI-50 scaffold or scaffold portion
guuugagaguuauggaaacaugacgaguucaaauaaaaauuuauucaaaccgccuauuuauaggccgcagauguucugcauuuugugcaagcuuuuuu

SEQ ID NO: 120 - RNA, 88 nt, Artificial Sequence
  OMNI-50 scaffold or scaffold portion
guuugagaguuauggaaacaugacgaguucaaauaaaaauuuauucaaaccgccuauuuauaggccgcaggcuauugcaagcuuuuuu

SEQ ID NO: 121 - RNA, 88 nt, Artificial Sequence
  OMNI-50 scaffold or scaffold portion
guuugagaguuauggaaacaugacgaguucaaauaaaaauuuauucaaaccgccuauuuauaggccgcagauguucugcagcuuuuuu

SEQ ID NO: 122 - RNA, 98 nt, Artificial Sequence
  OMNI-50 scaffold or scaffold portion
guuugagaguuauggaaacaugacgaguucaaauaaaaauuuauucaaaccgccuauuuauaggccgcagauguucugcauuaugcuuaagcuuuuuu

SEQ ID NO: 123 - RNA, 82 nt, Artificial Sequence
  OMNI-50 scaffold or scaffold portion
guuugagaguuauggaaacaugacgaguucaaauaaaaauuuauucaaaccgccuauuuauaggccgcagauguucuguuuu

SEQ ID NO: 124 - RNA, 84 nt, Artificial Sequence
  OMNI-50 scaffold or scaffold portion
guuugagaguuauggaaacaugacgaguucaaauaaaaauuuauucaaaccgccuauuuauaggccgcagauguucugcuuuuu

SEQ ID NO: 125 - RNA, 98 nt, Artificial Sequence
  OMNI-50 scaffold or scaffold portion
guuugagaguuauggaaacaugacgaguucaaauaaaaauuuauucaaaccgccuauuuauaggccgcagauguucugcugcuauugcaagcuuuuuu

SEQ ID NO: 126 - RNA, 80 nt, Artificial Sequence
  OMNI-50 scaffold or scaffold portion
guuugagaguuaugaaaaugacgaguucaaauaaaaauuuauucaaaccgccuauuuauaggccgcagauguucugcuuu

SEQ ID NO: 127 - RNA, 13 nt, Artificial Sequence
  OMNI-50 scaffold or scaffold portion
auuuauucaaacc

SEQ ID NO: 128 - RNA, 13 nt, Artificial Sequence
  OMNI-50 scaffold or scaffold portion
aaauauucaaacc

SEQ ID NO: 129 - RNA, 10 nt, Artificial Sequence
  OMNI-50 scaffold or scaffold portion
gauguucugc

SEQ ID NO: 130 - RNA, 10 nt, Artificial Sequence
  OMNI-50 scaffold or scaffold portion
cgcguucugc

SEQ ID NO: 131 - RNA, 13 nt, Artificial Sequence
  OMNI-50 scaffold or scaffold portion
ugcaagcuuuuuu

SEQ ID NO: 132 - RNA, 78 nt, Artificial Sequence
  OMNI-50 scaffold or scaffold portion
guuugagaguuagaaaugacgaguucaaauaaaaauuuauucaaaccgccuauuuauaggccuauugcaagcuuuuuu

SEQ ID NO: 133 - RNA, 97 nt, Artificial Sequence
  OMNI-50 scaffold or scaffold portion
guuugagaguuaugaaaaugacgaguucaaauaaaaauuuauucaaaccgccuauuuauaggccuucugcauuaugcuugcuauugcaagcuuuuuu

SEQ ID NO: 134 - RNA, 100 nt, Artificial Sequence
  OMNI-50 scaffold or scaffold portion
guuugagaguuauguaagaaauuacaugacgaguucaaauaaaaauuuauucaaaccgccuaugauguucugcauuaugcuugcuauugcaagcuuuuuu

SEQ ID NO: 135 - RNA, 80 nt, Artificial Sequence
  OMNI-50 scaffold or scaffold portion
guuugagaguuagaaaugacgaguucaaauaaaaauuuauucaaaccgccuauuuauaggccgcaguugcaagcuuuuuu

SEQ ID NO: 136 - RNA, 84 nt, Artificial Sequence
  OMNI-50 scaffold or scaffold portion
guuugagaguuagaaaugacgaguucaaauauuuauucaaaccgccuauuuauaggccgcagauguucugcugcaagcuuuuuu

SEQ ID NO: 137 - RNA, 98 nt, Artificial Sequence
  OMNI-50 scaffold or scaffold portion
guuugagaguuauggaaacaugacgaguucaaauaaauauucaaaccgccuauuuauaggccgcguucugcauuaugcuugcuauugcaagcuuuuuu

SEQ ID NO: 138 - RNA, 15 nt, Artificial Sequence
  OMNI-50 scaffold or scaffold portion
ugacgaguucaaaua

SEQ ID NO: 139 - RNA, 13 nt, Artificial Sequence
  OMNI-50 scaffold or scaffold portion
aaaaauucaaacc

SEQ ID NO: 140 - RNA, 13 nt, Artificial Sequence
  OMNI-50 scaffold or scaffold portion
aauuauucaaacc

SEQ ID NO: 141 - RNA, 13 nt, Artificial Sequence
  OMNI-50 scaffold or scaffold portion
aaaaauuuaaacc

SEQ ID NO: 142 - RNA, 13 nt, Artificial Sequence
  OMNI-50 scaffold or scaffold portion
aaaaauuuauucc

SEQ ID NO: 143 - RNA, 13 nt, Artificial Sequence
  OMNI-50 scaffold or scaffold portion
aaaaauuuauuca

SEQ ID NO: 144 - RNA, 13 nt, Artificial Sequence
  OMNI-50 scaffold or scaffold portion
ccuauuuauaggc

SEQ ID NO: 145 - RNA, 12 nt, Artificial Sequence
  OMNI-50 scaffold or scaffold portion
cuauuuauaggc

SEQ ID NO: 146 - RNA, 13 nt, Artificial Sequence
  OMNI-50 scaffold or scaffold portion
gccuauuuauagg

SEQ ID NO: 147 - RNA, 11 nt, Artificial Sequence
  OMNI-50 scaffold or scaffold portion
agauguucugc

SEQ ID NO: 148 - RNA, 102 nt, Artificial Sequence
  OMNI-50 scaffold or scaffold portion
guuugagaguuauggaaacaugacgaguucaaauaaaaauucaaaccgccuauuuauaggccgcagauguucugcauuaugcuugcuauugcaagcuuuuuu

SEQ ID NO: 149 - RNA, 102 nt, Artificial Sequence
  OMNI-50 scaffold or scaffold portion
guuugagaguuauggaaacaugacgaguucaaauaauuauucaaaccgccuauuuauaggccgcagauguucugcauuaugcuugcuauugcaagcuuuuuu

SEQ ID NO: 150 - RNA, 102 nt, Artificial Sequence
  OMNI-50 scaffold or scaffold portion
guuugagaguuauggaaacaugacgaguucaaauauuuauucaaaccgccuauuuauaggccgcagauguucugcauuaugcuugcuauugcaagcuuuuuu

SEQ ID NO: 151 - RNA, 92 nt, Artificial Sequence
  OMNI-50 scaffold or scaffold portion
guuugagaguuauggaaacaugacgaguucaaauaacgccuauuuauaggccgcagauguucugcauuaugcuugcuauugcaagcuuuuuu

SEQ ID NO: 152 - RNA, 102 nt, Artificial Sequence
  OMNI-50 scaffold or scaffold portion
guuugagaguuauggaaacaugacgaguucaaauaaauauucaaaccgccuauuuauaggccgcagauguucugcauuaugcuugcuauugcaagcuuuuuu

SEQ ID NO: 153 - RNA, 84 nt, Artificial Sequence
  OMNI-50 scaffold or scaffold portion
guuugagaguuauggaaacaugacgaguucaaauaaaaauggccgcagauguucugcauuaugcuugcuauugcaagcuuuuuu

SEQ ID NO: 154 - RNA, 102 nt, Artificial Sequence
  OMNI-50 scaffold or scaffold portion
guuugagaguuauggaaacaugacgaguucaaauaaaaauuuaaaccgccuauuuauaggccgcagauguucugcauuaugcuugcuauugcaagcuuuuuu

SEQ ID NO: 155 - RNA, 102 nt, Artificial Sequence
  OMNI-50 scaffold or scaffold portion
guuugagaguuauggaaacaugacgaguucaaauaaaaauuuauuccgccuauuuauaggccgcagauguucugcauuaugcuugcuauugcaagcuuuuuu

SEQ ID NO: 156 - RNA, 78 nt, Artificial Sequence
  OMNI-50 scaffold or scaffold portion
guuugagaguuagaaaugacgaguucaaauaaaaggccgcagauguucugcauuaugcuugcuauugcaagcuuuuuu

SEQ ID NO: 157 - RNA, 90 nt, Artificial Sequence
  OMNI-50 scaffold or scaffold portion
guuugagaguuauggaaacaugacgaguucaaauaaccuauuuauaggccgcagauguucugcauuaugcuugcuauugcaagcuuuuuu

SEQ ID NO: 158 - RNA, 94 nt, Artificial Sequence
  OMNI-50 scaffold or scaffold portion
guuugagaguuauggaaacaugacgaguucaaauaaaacgccuauuuauaggccgcagauguucugcauuaugcuugcuauugcaagcuuuuuu

SEQ ID NO: 159 - RNA, 88 nt, Artificial Sequence
  OMNI-50 scaffold or scaffold portion
guuugagaguuauggaaacaugacgaguucaaauacuauuuauaggccgcagauguucugcauuaugcuugcuauugcaagcuuuuuu

SEQ ID NO: 160 - RNA, 84 nt, Artificial Sequence
  OMNI-50 scaffold or scaffold portion
guuugagaguuauggaaacaugacgaguucaaauaauauaggccgcagauguucugcauuaugcuugcuauugcaagcuuuuuu

SEQ ID NO: 161 - RNA, 82 nt, Artificial Sequence
  OMNI-50 scaffold or scaffold portion
guuugagaguuauggaaacaugacgaguucaaauaauaggccgcagauguucugcauuaugcuugcuauugcaagcuuuuuu

SEQ ID NO: 162 - RNA, 102 nt, Artificial Sequence
  OMNI-50 scaffold or scaffold portion
guuugagaguuauggaaacaugacgaguucaaauaaaaauuuauucagccuauuuauaggccgcagauguucugcauuaugcuugcuauugcaagcuuuuuu

SEQ ID NO: 163 - RNA, 90 nt, Artificial Sequence
  OMNI-50 scaffold or scaffold portion
guuugagaguuaugaaaaugacgaguucaaauaccgccuauuuauaggccgcagauguucugcauuaugcuugcuauugcaagcuuuuuu

SEQ ID NO: 164 - RNA, 96 nt, Artificial Sequence
  OMNI-50 scaffold or scaffold portion
guuugagaguuauggaaacaugacgaguucaaauacaaaccgccuauuuauaggccgcagauguucugcauuaugcuugcuauugcaagcuuuuuu

SEQ ID NO: 165 - RNA, 102 nt, Artificial Sequence
  OMNI-50 scaffold or scaffold portion
guuugagaguuauggaaacaugacgaguucaaauaaaaauuuauucaaaccgccuauuuauaggagauguucugcauuaugcuugcuauugcaagcuuuuuu

SEQ ID NO: 166 - RNA, 78 nt, Artificial Sequence
  OMNI-50 scaffold or scaffold portion
guuugagaguuagaaaugacgaguucaaauaauaggccgcagauguucugcauuaugcuugcuauugcaagcuuuuuu

SEQ ID NO: 167 - RNA, 92 nt, Artificial Sequence
  OMNI-50 scaffold or scaffold portion
guuugagaguuauggaaacaugacgaguucaaauaaaaauuuauucaaaccgccugauguucugcauuaugcuugcuauugcaagcuuuuuu

SEQ ID NO: 168 - DNA, 22 bp, Artificial Sequence
  Spacer or target sequence
gcagtccgggctgggagcgggt

SEQ ID NO: 169 - DNA, 22 bp, Artificial Sequence
  Spacer or target sequence
cagctgcgggaaagggattccc

SEQ ID NO: 170 - DNA, 22 bp, Artificial Sequence
  Spacer or target sequence
cagctgcgggaatgggattccc

SEQ ID NO: 171 - DNA, 22 bp, Artificial Sequence
  Spacer or target sequence
gtgtcaagccccagaggccaca

SEQ ID NO: 172 - DNA, 22 bp, Artificial Sequence
  Spacer or target sequence
gtgtcaagccccagaggacaca

SEQ ID NO: 173 - DNA, 22 bp, Artificial Sequence
  Spacer or target sequence
cacagcgggtgtagactccgag

SEQ ID NO: 174 - DNA, 22 bp, Artificial Sequence
  Spacer or target sequence
cacagcaggtgtagactccgag

SEQ ID NO: 175 - DNA, 25 bp, Artificial Sequence
  Spacer or target sequence
gcagtccgggctgggagcgggtggg

SEQ ID NO: 176 - DNA, 25 bp, Artificial Sequence
  Spacer or target sequence
cagctgcgggaaagggattcccagg

SEQ ID NO: 177 - DNA, 25 bp, Artificial Sequence
  Spacer or target sequence
cagctgcgggaatgggattcccagg

SEQ ID NO: 178 - DNA, 25 bp, Artificial Sequence
  Spacer or target sequence
gtgtcaagccccagaggccacaggg

SEQ ID NO: 179 - DNA, 25 bp, Artificial Sequence
  Spacer or target sequence
gtgtcaagccccagaggacacaggg

SEQ ID NO: 180 - DNA, 25 bp, Artificial Sequence
  Spacer or target sequence
cacagcgggtgtagactccgagggg

SEQ ID NO: 181 - DNA, 25 bp, Artificial Sequence
  Spacer or target sequence
cacagcaggtgtagactccgagggg

SEQ ID NO: 182 - DNA, 25 bp, Artificial Sequence
  Spacer or target sequence
gcagacagggccaggagcgggaggg

SEQ ID NO: 183 - DNA, 25 bp, Artificial Sequence
  Spacer or target sequence
gtggggcgggaaagggagtcccagg

SEQ ID NO: 184 - DNA, 25 bp, Artificial Sequence
  Spacer or target sequence
cagctgagggaaaggaattcccagg

SEQ ID NO: 185 - DNA, 25 bp, Artificial Sequence
  Spacer or target sequence
aagccaaaccccaaaggccacacgg

SEQ ID NO: 186 - DNA, 25 bp, Artificial Sequence
  Spacer or target sequence
aagacaagacccagaggacacaggg

SEQ ID NO: 187 - DNA, 25 bp, Artificial Sequence
  Spacer or target sequence
acagtcctggctgggagcaggtggg

SEQ ID NO: 188 - DNA, 25 bp, Artificial Sequence
  Spacer or target sequence
cgcctgtgggaatgggcttccccgg

SEQ ID NO: 189 - DNA, 25 bp, Artificial Sequence
  Spacer or target sequence
tggccaagcctcagaggccacaggg

SEQ ID NO: 190 - DNA, 25 bp, Artificial Sequence
  Spacer or target sequence
tcttcaagccccagaggacactggg